(19) United States
(12) Patent Application Publication
(10) Pub. No.: US 2009/0214445 A1
(43) Pub. Date: Aug. 27, 2009

(54) DELIVERY SYSTEMS FOR MANAGING RELEASE OF FUNCTIONAL INGREDIENTS IN AN EDIBLE COMPOSITION

(75) Inventors: Navroz Boghani, Flanders, NJ (US); Petros Gebresellasie, Piscataway, NJ (US); Shiuh John Luo, Livingston, NJ (US)

Correspondence Address: OBLON, SPIVAK, MCCLELLAND MAIER & NEUSTADT, P.C., 1940 DUKE STREET, ALEXANDRIA, VA, 22314, US

(73) Assignee: Cadbury Adams USA LLC, Parsippany, NJ (US)

(21) Appl. No.: 11/913,267

(22) PCT Filed: May 19, 2006
(86) PCT No.: PCT/US06/19871

§ 371 Date: May 9, 2008

Related U.S. Application Data
(60) Provisional application No. 60/683,634, filed on May 23, 2005.
(60) Provisional application No. 60/734,680, filed on Nov. 8, 2005.

(30) Foreign Application Priority Data
May 23, 2005 (US) 11/134356
May 23, 2005 (US) 11/134364
May 23, 2005 (US) 11/134365
May 23, 2005 (US) 11/134367
May 23, 2005 (US) 11/134369
May 23, 2005 (US) 11/134370
May 23, 2005 (US) 11/134371
May 23, 2005 (US) 11/134480
May 23, 2005 (US) 11/135149
May 23, 2005 (US) 11/135153

(51) Int. Cl. A61K 9/68

(52) U.S. Cl. 424/48

(57) ABSTRACT

A delivery system for inclusion in an edible composition is formulated to have at least one ingredient encapsulated with an encapsulating material. The delivery system and the resulting edible composition may include other ingredients to create a desired release profile for the at least one ingredient.

### FIELD

The present invention is generally directed to a delivery system for an edible composition in which the release of at least one ingredient in the delivery system is managed for delivery to a consumer of the edible composition or for use with another ingredient in the edible composition. The delivery system and the resulting edible composition may include one or more other ingredients to create a desired release rate and/or release profile for the at least one ingredient and/or the other ingredient(s).

### BACKGROUND

Confectionery, beverages, and other edible compositions may provide one or more sensory experiences or other benefits to a consumer. For example, a chewing gum may provide a spicy flavor, a mouth cooling sensation, or other sensory experience to a consumer when the consumer chews the gum. As another example, a chewing gum may provide a therapeutic or other functional benefit such as teeth whitening, breath freshening, calcium delivery, etc., to a consumer when the consumer chews the gum.

In some edible compositions, it may be desirable to manage the release of the active in chewing gum or other edible composition such that a desired release rate or release profile of the active is at least partially obtained. For example, in a chewing gum it may be preferable to provide a longer or delayed delivery of a tooth whitening active at a lower concentration during chewing of the gum than a quicker or earlier delivery of higher concentration of the same active during chewing of the gum. As another example, in a chewing gum it may be preferable to release a high intensity sweetener (e.g., aspartame, sucralose, neotame) throughout the gum chewing process by a consumer or at least at intervals during the gum chewing process by a consumer.

It would therefore be a significant advance in the art to provide methods of producing and using delivery systems and/or one or more ingredients for edible compositions that allow for management of the release rate and/or profile for the one or more ingredients in the delivery system.

### SUMMARY

Delivery systems useful in edible compositions are disclosed herein. In some embodiments, a delivery system may include one or more ingredients (e.g., flavors, flavor potentiators, acids, mouth moisteners, colors, cooling agents, warming agents, sensates, actives, vitamins or other micronutrients, high intensity sweeteners, emulsifiers or surfactants, taste masking agents, oral care actives, breath freshening actives, minerals, cooling potentiators, warming potentiators, sweetness potentiators, throat soothing agents, mouth moistening agents, remineralization agents, demineralization agents, antibacterial agents, antimicrobial agents, anticalculus agents, bitterness masking agents) that are partially or completely encapsulated with an encapsulating material (e.g., water insoluble polymer or co-polymer).

In some embodiments, a delivery system or an edible composition that includes the delivery system as an ingredient may include one or more ingredients, amounts of one or more ingredients, or ratios of two or more ingredients, etc., such that the release rate or release profile of one or more of these ingredients, or another ingredient in the delivery system or edible composition, is managed during consumption or other use of the delivery system or edible composition.

As used herein, the term “beverage” includes any potable substance (including, but not limited to, carbonated soda drinks, non-carbonated drinks, bottled water, mineral and aerated waters, chocolate drinks, milk and milk-based drinks, fruit drinks and juices, teas, alcoholic drinks, non-alcoholic drinks, beers, wines, and coffees), or a product incorporating a potable substance, or a substance which, when combined with one or more other substances, becomes a potable substance. A beverage may exist in any suitable state, including but not limited to, a powdered, liquid, semi-liquid, or semi-frozen state. In some embodiments, a beverage may be a concentrate, a supplement, or some other preliminary form that may be added to another substance.

As used herein, the term “carrier” includes an orally acceptable vehicle, such as the soluble and insoluble components of a chewing gum composition, that is capable of being mixed with a delivery system, and which will not cause harm to warm-blooded animals including humans. Carriers further include those components of an edible composition that are capable of being comingled with a delivery system without significant interaction with the delivery system.

As used herein, the term “comprising” (also “comprises” etc.), is synonymous with “including,” “containing,” or “characterized by,” is inclusive and open-ended and does not exclude additional unrecited elements or method steps, regardless of its use in the description or in the preamble or body of a claim.

As used herein, the term “confectionery” includes, but is not limited to, chewing gum (which includes bubble gum), chocolate, lozenges, mints, tablets, chewy candies, hard candies, boiled candies, breath and other oral care films or strips, candy canes, lollipops, gummies, jellies, fudge, caramel, hard and soft panned goods, toffee, taffy, gelatin candies, gum drops, jelly beans, nougats, fondants, or combinations of one or more of these, or edible compositions incorporating one or more of these.

As used herein, the term “delivery system” includes an encapsulating material and at least one ingredient encapsulated with the encapsulating material. In some embodiments, a delivery system may include multiple ingredients, multiples layers or levels of encapsulation, and/or one or more other additives. A delivery system may be an ingredient in an edible composition. In some embodiments, the one or more ingredients and an encapsulating material in the delivery system may form a matrix. In some embodiments, the encapsulating material may completely coat or cover the one or more ingredients or form a partial or complete shell, cover, or coating around the ingredients.

As used herein, the term “edible composition” includes, but is not limited to, beverages, confectionery compositions and products, food compositions and products, etc. An edible composition may include one or more delivery systems as one of its ingredients. Each delivery system in an edible composition may have the same or different ingredients, the same or different encapsulating materials, and/or the same or different characteristics (e.g., tensile strength, water solubility, ratio of ingredient to encapsulating material, average or maximum particle size of ingredient, average or maximum particle size of ground delivery system, distribution of particle sizes of the ground delivery system, ratio of different polymers used to encapsulate one or more ingredients, hydrophobicity of one or more polymers used to encapsulate one or more ingredients, hydrophobicity of the delivery system, coating on the delivery system, coating on an ingredient prior to the ingredient being encapsulated). One or more of these characteristics may be used to define or characterize the release profile for one or more ingredients when the one or more ingredients are in an edible composition. In addition, in some embodiments, an edible composition may include multiple delivery systems, each of which includes the same or similar ingredients encapsulated in a different way and/or with a different encapsulating material. In some embodiments, the edible composition also might include free (i.e., unencapsulated) amounts of one or more ingredients. The free ingredient(s) may be one or more of the same ingredients present in a delivery system that also is used in the edible composition.

As used herein, the term “encapsulating material” includes any one or more water insoluble polymers, co-polymers, or other materials capable of forming a coating, shell, or film as a protective barrier or layer around one or more ingredients and/or capable of forming a matrix with the one or more ingredients. In some embodiments, the encapsulating material may completely surround, coat, cover, or enclose an ingredient. In other embodiments, the encapsulating material may only partially surround, coat, cover, or enclose an ingredient.

As used herein, the term “tensile strength” includes the maximum stress a material subjected to a stretching load can withstand without tearing. A standard method for measuring tensile strength of a given substance is defined by the American Society of Testing Materials in method number ASTM-D638.

In some embodiments, there is a delivery system for use in an edible composition, the delivery system including: an encapsulating material; and a functional ingredient encapsulated with the encapsulating material, the encapsulated functional ingredient having a tensile strength greater than about 6500.

In some embodiments, there is a delivery system for use in an edible composition, the delivery system including: an encapsulating material; and a functional ingredient encapsulated with the encapsulating material, wherein the encapsulating material has a hydrophobicity as measured by water absorption of 0 to 15% by weight.

In some embodiments, there is a delivery system for use in an edible composition, the delivery system including: an encapsulating material; and a functional ingredient encapsulated with the encapsulating material, wherein the encapsulating material has a hydrophobicity as measured by water absorption of 15 to 50% by weight.

In some embodiments, there is a delivery system for use in an edible composition, the delivery system including: an encapsulating material; and a functional ingredient encapsulated with the encapsulating material, wherein the encapsulating material has a hydrophobicity as measured by water absorption of 50 to 100% by weight.

In some embodiments, there is a delivery system for use in an edible composition, the delivery system including: an encapsulating material; and a functional ingredient encapsulated with the encapsulating material, wherein the delivery system has an average particle size less than about 710 microns.

In some embodiments, an edible composition includes: a first delivery system containing a first encapsulating material and a functional ingredient encapsulated with the first encapsulating material; and a second delivery system containing a second encapsulating material and an ingredient encapsulated with the second encapsulating material.

### BRIEF DESCRIPTION OF THE DRAWINGS

FIG. 1 shows the amounts of sucralose remaining in a chewed gum bolus for five time points (from 0 to 20 minutes) for chewing gums made with free sucralose as compared to chewing gums made with two different sucralose encapsulations.

FIG. 2 shows the amount of aspartame remaining in a chewed gum bolus for three time points (from 10 to 30 minutes) for chewing gums made with free aspartame as compared to chewing gums made with encapsulated aspartame.

FIG. 3 shows the amount of sodium tripolyphosphate (STP) remaining in a chewed gum bolus for five time points (from 0 to 20 minutes) for chewing gums made with free STP as compared to chewing gums made with encapsulated STP.

### DETAILED DESCRIPTION

An ingredient in an edible composition will have a release profile when a consumer consumes the edible composition. In some embodiments, the ingredient may be released by mechanical action of the chewing, and/or by chemical action or reaction of the ingredient with another ingredient or saliva or other material in the consumer's mouth. The release profile for the ingredient is indicative of the availability of the ingredient in the consumer's mouth to interact with receptors (e.g., taste receptors), mucous membranes, teeth, etc. in the consumer's mouth. An edible composition may include the same or different release profiles for different ingredients. In some embodiments, the release profile for only a finite number (e.g., one or two) ingredients may be of primary importance.

The release profile of an ingredient in an edible composition can be influenced by many factors such as, for example, rate of chewing, intensity of chewing, amount of the ingredient in the edible composition, the form of the ingredient added to the edible composition (e.g., encapsulated in a delivery system, unencapsulated, pretreated), how the edible composition is mixed or otherwise prepared, when or how the ingredient is added to other ingredients in the edible composition, the ratio of the amount of the ingredient to the amount(s) of one or more other ingredients in the edible composition, the ratio of the amount of the ingredient to the amount of one or more other ingredients in a delivery system that is included in the edible composition, etc.

In some embodiments, a release profile for an ingredient may relate to a specific time period. For example, release of an ingredient from a delivery system may increase during a first time period, reach a peak, and then decrease during a second time period. Thus, in some embodiments, a release profile for an ingredient may include one or more time periods, each of which has an associated release rate (which may or may not be known or measurable). The time periods may be the same length of time or may be different lengths of time. A first time period may have a fixed or varied release rate for the ingredient during the first time period and an average release rate for the ingredient over the first time period. Similarly, a second time period may have a fixed or varied release rate for the ingredient during the second time period and an average release rate for the ingredient over the second time period. In some embodiments, a release profile for an ingredient in an edible composition may include only one time period or be related to only a single point in time, both of which typically relate or are relative to when consumption of the edible composition has started. In other embodiments, a release profile may relate to two or more time periods and/or two or more points in time, all of which typically relate or are relative to when consumption of the edible product has started.

In some embodiments, a release profile may be defined or characterized by one or more factors or characteristics, even if other or all aspects of the release profile are not determined, selected, or even known. Thus, in some embodiments, a release profile for an ingredient may include only one characteristic. For example, characteristics may include one or more of the following: release rate of an ingredient during a time period, a specific time period during which a minimum, average, or predominant amount of an ingredient is released during consumption of an edible composition that includes the ingredient (even if some of the ingredient is released before or after the specific time period and even if the release rate during the time period is not specified or varies), a specific time after which a minimum, average, or predominant amount if an ingredient is released during consumption of an edible composition that includes the ingredient (even if some of the ingredient is released before the specific time and even if the release rates are or are not specified), etc.

In some embodiments, managing a release profile for one or more ingredients may include changing or otherwise managing the starting and ending times for the time periods, changing or otherwise managing the lengths of the time periods, and/or changing or otherwise managing the release rates during the time periods. For example, managing a release profile may include changing or managing a release rate during a time period. An ingredient can be released more quickly or earlier during a first or second time period by increasing its release rate during these time periods. Likewise, the ingredient can be released more slowly or in a more delayed manner during the first or second time periods by decreasing its release rate during these time periods. As another example, managing a release profile may include shifting the start and end of the time periods in the release profile, but the length of the time periods may stay the same and the release rates of the ingredient(s) during the time periods may stay the same (e.g., the release of an ingredient may be managed to delay the release of the predominant amount of the ingredient by one minute, five minutes, ten minutes, thirty minutes, etc.). As a third example, managing a release profile may include shifting the start or end of one or more time periods and changing the release rate within the one or more time periods.

In some embodiments, causing a delay in a release of an ingredient in an edible composition includes causing a delay in the release or availability of the predominant amount of the ingredient after consumption of the edible product begins and/or causing release or availability of a desired, predominant, or minimum amount of the ingredient at a certain time, after a certain time, or during a desired time period after consumption of the edible composition begins. In some embodiments, none of the ingredient will be released or become available before the certain time or before or after the desired time period. In other embodiments, some of the ingredient may be released or become available before the certain time and/or before or after the desired time period.

In some embodiments, determining or selecting a desired release profile may include determining or selecting one or more factors or characteristics of the desired release profile, as previously described above. The factors or characteristics then serve to define or characterize the release profile, even if other or all aspects of the release profile are not determined or selected. Thus, determining or selecting a release profile for an ingredient can include situations where only one characteristic for the release of the ingredient is determined or selected. In some embodiments, characteristic may be determined or measured by one or more techniques or methods such as, for example, chemical and/or mechanical testing and analysis, consumer testing, descriptive or expert taste or chew panel, other in vivo or in vitro testing, etc.

Applicants have recognized that management of the release rate or release profile of one or more ingredients in an edible composition (e.g., beverages, confectionery) may allow for improved edible compositions. For example, management of a release rate or release profile of one or more ingredients in an edible composition may allow better or more complete delivery of one or more ingredients in an edible composition to a consumer of the edible composition, thereby improving the edible composition. In general, in some embodiments, this may result in improved consumer acceptance of the edible composition, reduced costs to produce the edible composition, improved efficacy of the edible composition, or other benefits. More specifically, in some embodiments the improvement may lie in increased stability of the edible composition or an ingredient in the edible composition, increased efficacy of the edible composition when used or consumed by a consumer, increased duration in sensory experience (e.g., flavor, texture, mouth cooling or warming sensation, nasal cooling sensation) provided to a consumer of the edible composition, decreased impact of a negative attribute of the edible composition (e.g., less bitterness or bad taste) during consumption of the edible composition, etc.

As a more specific example, managing the release profile of a high intensity sweetener or flavor provided by a chewing gum when a consumer chews the chewing gum may increase the duration of flavor provided to the consumer, or at least the perception of an improvement in duration of the flavor delivery by the chewing gum to the consumer.

There are many reasons why management of the release rate or release profile of one or more ingredients in an edible composition may be desirable. One or more different reasons may apply to different ingredients, delivery systems, and/or edible compositions. For example, in some embodiments, it may be desired to delay the release of an ingredient during consumption of an edible composition that contains the ingredient. If early and extended release of the ingredient is desired, the edible composition may include free amounts of the ingredient, as well as encapsulated amounts of the ingredient in one or more delivery systems (which may be the same or different), to create a desired release profile of the ingredient. In some embodiments, a free ingredient may be used to deliver an initial amount or “hit” of an ingredient (e.g., flavor, cooling agent) or an initial sensation or benefit caused by the ingredient (e.g., flavor, nasal action, cooling, warming, tingling, saliva generation, breath freshening, throat soothing, mouth moistening). The encapsulated portion of the ingredient may then provide an additional or delayed amount of the same sensation or benefit. By using both the free ingredient and the encapsulated ingredient, the sensation or benefit may be provided over a longer period of time and/or perception of the sensation or benefit by a consumer may be improved. Also, the initial amount or “hit” of the ingredient may predispose or precondition the consumers' mouth or perception of the edible composition.

As another example, in some embodiments it may be desired to provide a sustained release of an ingredient in an edible composition over time. The sustained release may allow for a lower concentration of the ingredient to be released over a longer period of time versus the release of a higher concentration of the ingredient over a shorter period of time. A sustained release of an ingredient may be advantageous in situations when the ingredient has a bitter or other bad taste at the higher concentrations. A sustained release of an ingredient also may be advantageous when release of the ingredient in higher concentrations over a shorter period of time may result in a lesser amount of the ingredient being optimally delivered to the consumer. For example, for a tooth whitening or breath freshening ingredient, providing too much of the ingredient too fast may result in a consumer swallowing a significant portion of the ingredient before the ingredient has had a chance to interact with the consumer's teeth, mucous membranes, and/or dental work, thereby wasting the ingredient or at least reducing the benefit of having the ingredient in the edible composition.

As another example, in some embodiments it may be desired to provide a change of sensory experience for a consumer of an edible composition during consumption of the product. More specifically, a chewing gum or lozenge may change predominant flavor from a fruit flavor to mint flavor or change color from red to blue as the consumer chews them. Alternatively, the chewing gum or lozenge may change the location in the consumer's mouth or throat where the consumer has a primary cooling or warming sensation, e.g., from the front of the mouth to the back of the mouth or to the nasal passages. As another example, the chewing gum or lozenge may provide a tingling sensation and/or a boost in flavor delivery or perception at some time (e.g., after ten minutes, after thirty minutes) during chewing of the edible composition. As another example, in some embodiments, a chewing gum may include ingredients that effervesce or form an effervescent system, such as an edible acid and a base, which react upon chewing to generate effervescence. One or both of the edible acid and the base may be encapsulated to delay their reaction and, as a result, the effervescence.

As another example, in some embodiments it may be desired to provide an indicator to a consumer of an edible composition via color change, flavor change, cooling sensation change, etc. Such a change may provide an indication to the consumer that the consumer has chewed the edible composition long enough to obtain a certain level of benefit provided by the edible composition (e.g., caffeine delivery, nicotine delivery, germ killing active delivery, tooth whitening active delivery).

As another example, in some embodiments it may be desired to release different ingredients in an edible composition from the edible composition at different times during consumption of the edible composition. As a more specific example, in a chewing gum directed to tooth whitening, the chewing gum may include a delivery system that has an ingredient that functions primarily as an abrasive or mechanical teeth cleaner. The chewing gum also may include a delivery system that has an ingredient that functions as a chemical teeth cleaner. While it may be beneficial to have some of each ingredient released into the consumer's mouth during the entire time the consumer is chewing the gum, it also may be beneficial to manage the release of the different cleaning ingredients so that the predominant amount of each ingredient is released at a different or desired time as the consumer chews the gum.

As another example, in some embodiments it may be desired to release multiple ingredients in an edible composition in a particular order during consumption of the edible composition.

As another example, in some embodiments, it may be desired to create a release profile for one or more ingredients in an edible product to assist in marketing or selling the product to a particular demographic segment or market (e.g., teenagers, people trying to quit smoking) or for a particular usage situation (e.g., after dinner breath freshening, energy enhancer or stimulator).

As another example, in some embodiments it may be desired to release two or more ingredients together, but in a delayed manner. For example, in a chewing gum there may be advantages to releasing one or more high intensity sweeteners (e.g., neotame, sucralose, aspartame, acesulfame-K (also referred to as “aceK” or “ace-K”)) along with a tooth whitening ingredient (e.g., pyrophosphates, triphosphates, polyphosphates, polyphosphonates, sodium hexametaphosphate, sodium tripolyphosphate, peroxide(s), proteolytic enzyme(s), and surfactants such as medium or long chain fatty acids) over time. Thus, the chewing gum may include free amounts of the high intensity sweetener(s) and tooth whitening ingredient as well as one or more delivery systems that include the high intensity sweetener(s) and the tooth whitening ingredient. The delivery system may be designed or otherwise selected to delay the release of a predominant amount of the high intensity sweetener(s) and the tooth whitening ingredient during chewing of the gum. If the chewing gum includes different delivery systems, the different delivery systems may delay the release of their associated high intensity sweetener(s) and the tooth whitening ingredient such that an overall release profile of the high intensity sweetener(s) and the tooth whitening ingredient is approximated or obtained.

There are many types of ingredients for which managed release of the ingredients from an edible composition during consumption of the edible composition may be desired. In addition, there are many groups of two or more ingredients for which managed release of the group of ingredients from an edible composition during consumption of the edible composition may be desired.

Types of individual ingredients for which managed release from an edible composition may be desired, include, but are not limited to the ingredients and combinations of ingredients described below. Ingredients may be different forms such as, for example, liquid form, spray-dried form, or crystalline form. In some embodiments, a delivery system or edible composition may include the same type of ingredient in different forms. For example, a chewing gum may include a liquid flavor and a spray-dried version of the same flavor.

### Ingredients—Actives

In some embodiments, the release profile of one or more actives can be managed. Actives generally refer to those ingredients that are included in a delivery system and/or edible composition for the desired end benefit they provide to the user. In some embodiments, actives can include medicaments, nutrients, nutraceuticals, herbals, nutritional supplements, pharmaceuticals, drugs, and the like and combinations thereof.

Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parlinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometliosis management agents, enzymes, erectile dysfunction therapies such as sildenafil citrate, which is currently marketed as Viagra™, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of active ingredients contemplated for use in the present invention can include antacids, H2-antagonists, and analgesics. For example, antacid dosages can be prepared using the ingredients calcium carbonate alone or in combination with magnesium hydroxide, and/or aluminum hydroxide. Moreover, antacids can be used in combination with H2-antagonists.

Analgesics include opiates and opiate derivatives, such as Oxycontin™, ibuprofen, aspirin, acetaminophen, and combinations thereof that may optionally include caffeine.

Other drug active ingredients for use in embodiments can include anti-diarrheals such as Immodium™ AD, anti-histamines, anti-tussives, decongestants, vitamins, and breath fresheners. Also contemplated for use herein are anxiolytics such as Xanax™; anti-psychotics such as Clozaril™ and Haldol™; non-steroidal anti-inflammatories (NSAID's) such as ibuprofen, naproxen sodium, Voltaren™ and Lodine™, anti-histamines such as Claritin™, Hismanal™, Relafen™, and Tavist™; anti-emetics such as Kytril™ and Cesamet™; bronchodilators such as Bentolin™, Proventil™; anti-depressants such as Prozac™, Zoloft™, and Paxil™; anti-migraines such as Inigram, ACE-inhibitors such as Vasotec™, Capoten™ and Zestril™; anti-Alzheimer's agents, such as Nicergoline™; and CaH-antagonists such as Procardia™, Adalat™, and Calan™.

The popular H2-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients can include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

A variety of nutritional supplements may also be used as active ingredients including virtually any vitamin or mineral. For example, vitamin A, vitamin C, vitamin D, vitamin E, vitamin K, vitamin B₆, vitamin B₁₂, thiamine, riboflavin, biotin, folic acid, niacin, pantothenic acid, sodium, potassium, calcium, magnesium, phosphorus, sulfur, chlorine, iron, copper, iodine, zinc, selenium, manganese, choline, chromium, molybdenum, fluorine, cobalt and combinations thereof, may be used.

Examples of nutritional supplements that can be used as active ingredients are set forth in U.S. Patent Application Publication Nos. 2003/0157213 A1, 2003/0206993 and 2003/0099741 A1 which are incorporated in their entirety herein by reference for all purposes.

Various herbals may also be used as active ingredients such as those with various medicinal or dietary supplement properties. Herbals are generally aromatic plants or plant parts and or extracts thereof that can be used medicinally or for flavoring. Suitable herbals can be used singly or in various mixtures. Commonly used herbs include Echinacea, Goldenseal, Calendula, Rosemary, Thyme, Kava Kava, Aloe, Blood Root, Grapefruit Seed Extract, Black Cohosh, Ginseng, Guarana, Cranberry, Ginko Biloba, St. John's Wort, Evening Primrose Oil, Yohimbe Bark, Green Tea, Ma Huang, Maca, Bilberry, Lutein, and combinations thereof.

Illustrations of the encapsulation of actives can be found in examples 15, 64, 114, and 164 provided herein. Typically, encapsulation of the active will result in a delay in the release of the predominant amount of the active during consumption of an edible composition that includes the encapsulated active (e.g., as part of a delivery system added as an ingredient to the edible composition). In some embodiments, the release profile of the ingredient (e.g., the active) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for an active include the intensity, duration, and type of mixing, the order of addition of ingredients to the mixing process, the positioning or location of ingredients in the final edible composition (e.g., whether or not layers or coating of ingredients are created), etc.

### Ingredients—Effervescing Systems

In some embodiments, the release profiles of one or more components of an effervescing system are managed. The effervescent system may include one or more edible acids and one or more bases. The edible acid(s) and the edible base(s) may react together to generate effervescence.

In some embodiments, the base(s) may be selected from, but is not limited to, alkali metal carbonates, alkali metal bicarbonates, alkaline earth metal carbonates, alkaline earth metal bicarbonates, and combinations thereof. The edible acid(s) may be selected from, but is not limited to, citric acid, phosphoric acid, tartaric acid, malic acid, ascorbic acid, and combinations thereof. In some embodiments, an effervescing system may include one or more other ingredients such as, for example, carbon dioxide, oral care ingredients, flavorants, etc.

For examples of use of an effervescing system in a chewing gum, refer to U.S. Provisional Patent No. 60/618,222 filed Oct. 13, 2004, and entitled “Effervescent Pressed Gum Tablet Compositions,” the contents of which are incorporated herein by reference for all purposes. Other examples can be found in U.S. Pat. No. 6,235,318, the contents of which are incorporated herein by reference for all purposes.

Typically, encapsulation of the one or more ingredients in an effervescing system will result in a delay in the release of the predominant amount of the one or more ingredients during consumption of an edible composition that includes the encapsulated one or more ingredients (e.g., as part of a delivery system added as an ingredient to the edible composition). The release profile of the one or more ingredients can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for one or more ingredients in an effervescing system include the intensity, duration, and type of mixing, the order of addition of ingredients to the mixing process, the positioning or location of ingredients in the final edible composition (e.g., whether or not layers or coating of ingredients are created), etc.

### Ingredients—Appetite Suppressors

In some embodiments, the release profiles of one or more appetite suppressors are managed. Appetite suppressors can be ingredients such as fiber and protein that function to depress the desire to consume food. Appetite suppressors can also include benzphetamine, diethylpropion, mazindol, phendimetrazine, phentermine, hoodia extracts (e.g., hoodia P57), Olibra,™ ephedra, caffeine and combinations thereof. Appetite suppressors are also known by the following trade names: Adipex,™ Adipost,™ Bontril™ PDM, Bontril™ Slow Release, Didrex,™ Fastin,™ Ionamin,™ Mazanor,™ Melfiat,™ Obenix,™ Phendiet,™ Phendiet-105,™ Phentercot,™ Phentride,™ Plegine,™ Prelu-2,™ Pro-Fast,™ PT 105,™ Sanorex,™ Tenuate,™ Sanorex,™ Tenuate,™ Tenuate Dospan,™ Tepanil Ten-Tab,™ Teramine,™ and Zantryl.™ These and other suitable appetite suppressors are further described in the following U.S. patents, all of which are incorporated in their entirety by reference hereto: U.S. Pat. No. 6,838,431 to Portman, U.S. Pat. No. 6,716,815 to Portman, U.S. Pat. No. 6,558,690 to Portman, U.S. Pat. No. 6,468,962 to Portman, U.S. Pat. No. 6,436,899 to Portman.

Illustrations of the encapsulation of appetite suppressors can be found in examples 15, 64, 114, and 164 provided herein. Typically, encapsulation of the appetite suppressor will result in a delay in the release of the predominant amount of the appetite suppressor during consumption of an edible composition that includes the encapsulated appetite suppressor (e.g., as part of a delivery system added as an ingredient to the edible composition). In some embodiments, the release profile of the ingredient (e.g., the appetite suppressor) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for an appetite suppressor include the intensity, duration, and type of mixing, the order of addition of ingredients to the mixing process, the positioning or location of ingredients in the final edible composition (e.g., whether or not layers or coating of ingredients are created), etc.

### Ingredients—Breath Fresheners

In some embodiments, the release profiles of one or more breath fresheners are managed. Breath fresheners can include essential oils as well as various aldehydes, alcohols, and similar materials. In some embodiments, essential oils can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde can be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments breath fresheners can include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, and combinations thereof. In some embodiments, the release profiles of probiotics can be managed including, but not limited to lactic acid producing microorganisms such as Bacillus coagulans, Bacillus subtilis, Bacillus laterosporus, Bacillus laevolacticus, Sporolactobacillus inulinus, Lactobacillus acidophilus, Lactobacillus curvatus, Lactobacillus plantarun, Lactobacillus jenseni, Lactobacillus casei, Lactobacillus femmentum, Lactococcus lactis, Pedioccocus acidilacti, Pedioccocus pentosaceus, Pedioccocus urinae, Leuconostoc mesenteroides, Bacillus coagulans, Bacillus subtilis, Bacillus laterosporus, Bacillus laevolacticus, Sporolactobacillus inulinus and mixtures thereof. Breath fresheners are also known by the following trade names: Retsyn,™ Actizol,™ and Nutrazin.™ Examples of malodor-controlling compositions are also included in U.S. Pat. No. 5,300,305 to Stapler et al. and in U.S. Patent Application Publication Nos. 2003/0215417 and 2004/0081713 which are incorporated in their entirety herein by reference for all purposes.

Illustrations of the encapsulation of breath freshening agents can be found in examples 18, 67, 7, 56, 14, 63, 103, 111, 153, and 161 provided herein. Typically, encapsulation of the breath freshening agent will result in a delay in the release of the predominant amount of the active during consumption of an edible composition that includes the encapsulated breath freshening agent (e.g., as part of a delivery system added as an ingredient to the edible composition). In some embodiments, the release profile of the ingredient (e.g., the breath freshening agent) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for an active include the intensity, duration, and type of mixing, the order of addition of ingredients to the mixing process, the positioning or location of ingredients in the final edible composition (e.g., whether or not layers or coating of ingredients are created), etc.

### Ingredients—Dental Care

In some embodiments, the release profiles of one or more oral care ingredients may be managed. Such oral care ingredients may include but are not limited to tooth whiteners, stain removers, oral cleaning, bleaching agents, desensitizing agents, dental remineralization agents, antibacterial agents, anticaries agents, plaque acid buffering agents, surfactants and anticalculus agents. Non-limiting examples of such ingredients can include, hydrolytic agents including proteolytic enzymes, abrasives such as hydrated silica, calcium carbonate, sodium bicarbonate and alumina, other active stain-removing components such as surface-active agents, including, but not limited to anionic surfactants such as sodium stearate, sodium palminate, sulfated butyl oleate, sodium oleate, salts of fumaric acid, glycerol, hydroxylated lecithin, sodium lauryl sulfate and chelators such as polyphosphates, which are typically employed as tartar control ingredients. In some embodiments, oral care ingredients can also include tetrasodium pyrophosphate and sodium tri-polyphosphate, sodium bicarbonate, sodium acid pyrophosphate, sodium tripolyphosphate, xylitol, and sodium hexametaphosphate.

In some embodiments, peroxides such as carbamide peroxide, calcium peroxide, magnesium peroxide, sodium peroxide, hydrogen peroxide, and peroxydiphospate are included. In some embodiments, potassium nitrate and potassium citrate are included. Other examples can include casein glycomacropeptide, calcium casein peptone-calcium phosphate, casein phosphopeptides, casein phosphopeptide-amorphous calcium phosphate (CPP-ACP), and amorphous calcium phosphate. Still other examples can include papaine, krillase, pepsin, trypsin, lysozyme, dextranase, mutanase, glycoamylase, amylase, glucose oxidase, and combinations thereof.

Further examples can include surfactants such as sodium stearate, sodium ricinoleate, and sodium lauryl sulfate surfactants for use in some embodiments to achieve increased prophylactic action and to render the oral care ingredients more cosmetically acceptable. Surfactants can preferably be detersive materials which impart to the composition detersive and foaming properties. Suitable examples of surfactants are water-soluble salts of higher fatty acid monoglyceride monosulfates, such as the sodium salt of the monosulfated monoglyceride of hydgrogenated coconut oil fatty acids, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine.

In addition to surfactants, oral care ingredients can include antibacterial agents such as, but not limited to, triclosan, chlorhexidine, zinc citrate, silver nitrate, copper, limonene, and cetyl pyridinium chloride. In some embodiments, additional anticaries agents can include fluoride ions or fluorine-providing components such as inorganic fluoride salts. In some embodiments, soluble alkali metal salts, for example, sodium fluoride, potassium fluoride, sodium fluorosilicate, ammonium fluorosilicate, sodium monofluorophosphate, as well as tin fluorides, such as stannous fluoride and stannous chloride can be included. In some embodiments, a fluorine-containing compound having a beneficial effect on the care and hygiene of the oral cavity, e.g., diminution of enamel solubility in acid and protection of the teeth against decay may also be included as an ingredient. Examples thereof include sodium fluoride, stannous fluoride, potassium fluoride, potassium stannous fluoride (SnF.sub.2-KF), sodium hexafluorostannate, stannous chlorofluoride, sodium fluorozirconate, and sodium monofluorophosphate. In some embodiments, urea is included.

Further examples are included in the following U.S. patents and U.S. published patent applications, the contents of all of which are incorporated in their entirety herein by reference for all purposes: U.S. Pat. Nos. 5,227,154 to Reynolds, 5,378,131 to Greenberg, 6,846,500 to Luo et al., 6,733,818 to Luo et al., 6,696,044 to Luo et al., 6,685,916 to Holme et al., 6,485,739 to Luo et al., 6,479,071 to Holme et al., 6,471,945 to Luo et al., U.S. Patent Publication Nos. 20050025721 to Holme et al., 2005008732 to Gebreselassie et al., and 20040136928 to Holme et al.

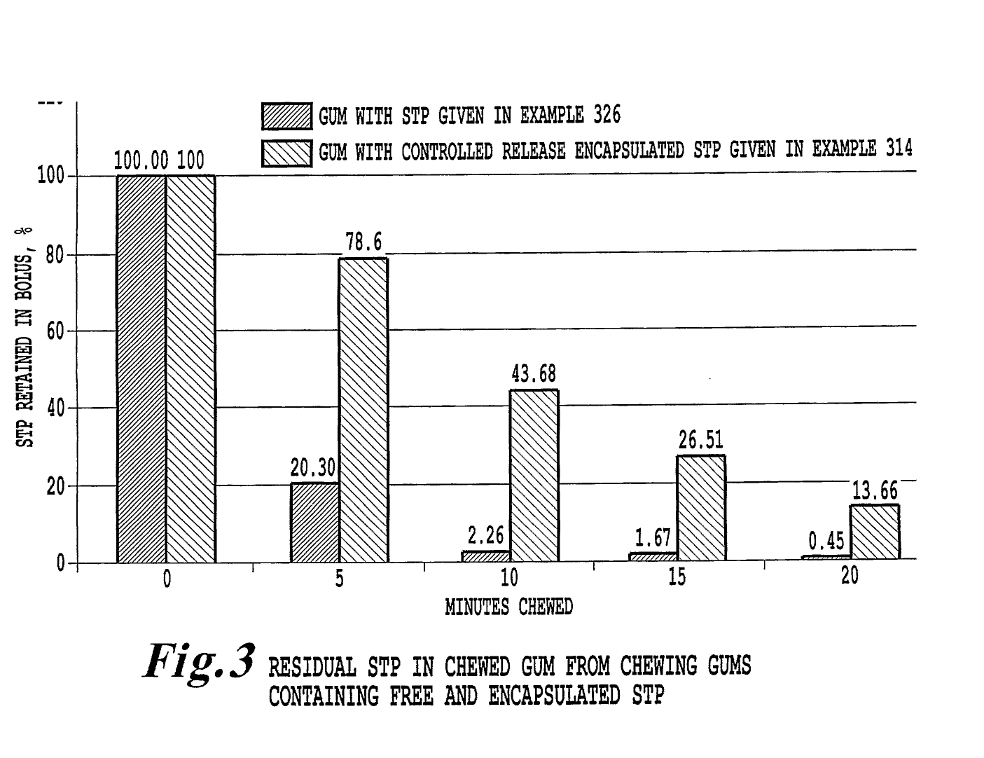

Illustrations of the encapsulation of dental care actives can be found in examples 300 through 326 inclusive, 350 through 377 inclusive, and FIG. 3 provided herein. Typically, encapsulation of the active will result in a delay in the release of the predominant amount of the active during consumption of an edible composition that includes the encapsulated active (e.g., as part of a delivery system added as an ingredient to the edible composition). In some embodiments, the release profile of the ingredient (e.g., the dental care active) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a dental care active include the intensity, duration, and type of mixing, the order of addition of ingredients to the mixing process, the positioning or location of ingredients in the final edible composition (e.g., whether or not layers or coating of ingredients are created), etc.

### Ingredients—Emulsifiers

In some embodiments, the release profiles of one or more emulsifiers may be managed. Emulsifiers can include molecules that have both a hydrophilic part and a hydrophobic part. Emulsifiers can operate at the interface between hydrophilic and hydrophobic materials. In some embodiments, an emulsifier may be selected or desired for use in a delivery system or edible composition based on one or more of its characteristics such as, for example, HLB value. For example, in some embodiments, an encapsulated composition may include a core material including at least one surfactant or other emulsifier having HLB of about seven or greater and an exterior coating encapsulating the core, the exterior coating including a material selected from cellulose, cellulose derivatives, starches, carbohydrates, gums, polyolefins, polyesters, waxes, vinyl polymers, gelatin, zein and combinations thereof. Some or all of the surfactant may be encapsulated. In some embodiments, a delivery system may include at least one active and at least one surfactant having an LB of seven or greater. The delivery system may be encapsulated as described herein. A chewing gum composition may include at least one flavor and at least one surfactant having an LB of about seven or greater. In some embodiments, the surfactant also may have an LB of about twenty or less. Some or all of the flavor and/or some or all of the surfactant may be encapsulated. The surfactants may be selected from a wide range of surfactants, particularly food grade surfactants, which are known in the art. The surfactant may have an HLB that is greater than about seven. More specifically, the surfactant will have an HLB of about fifteen or less, and even more specifically from about ten to about fourteen or from about eleven to thirteen.

Examples of useful surfactants include, but are not limited to, polyglycerol esters, ceteareth-20, sorbitan monostearate (Polysorbate 60), sorbitan monooleate (Polysorbate 80), sorbitan laurate (Polysorbate 20), sorbitan tristearate (Polysorbate 65), polyglyceryl laurate, glyceryl cocoate, acacia gum, acetylated monoglyceride, and combinations thereof. Polyglycerol esters include triglyceryl monostearate, hexaglyceryl distearate, decaglyceryl monostearate, decaglyceryl dipalmitate, decaglyceryl monooleate, and polyglyceryl 10 hexaoleate.

The surfactant and any other desired active may be combined with an encapsulating polymer by melt extrusion. This is conducted by melting a combination of one or more polymers in combination with the chosen surfactant(s) in the temperature range of about 65° C. to about 140° C. An active as described above may be added prior to melting the combination. The extrudate is then cooled and formed into particles of a desired size. This may be accomplished through cutting, grinding, pulverizing, milling or any other appropriate technique as know in the art. The extrudate particles may have an average particle size ranging from about 50 μm to about 800 μm.

The encapsulated surfactant particles of some embodiments may also be prepared by any suitable spray coating method as known in the art. One suitable process is the Wurster process. This process provides a method for encapsulating individual particulate materials. First the surfactant to be encapsulated (optionally in combination with an active) is suspended in a fluidizing air stream that provides a generally cyclic flow in front of a spray nozzle. The spray nozzle sprays an atomized flow of the coating solution which will include the encapsulating material in a suitable solvent. The atomized coating solution collides with the surfactant particles as they are carried away from the nozzle to provide a particle coating with the coating solution.

The temperature of the fluidizing air stream, which also serves to suspend the particles to be coated, may be adjusted to evaporate the solvent shortly after the coating solution contacts the particles. This serves to solidify the coating on the particles, resulting in the desired encapsulated particle.

This process may be repeated until the desired thickness of the coating is achieved. Alternatively, the process may be repeated with a different coating solution to provide different and distinct coating layers in the encapsulated particle composition.

Following the coating process, the particles may then be formed to an appropriate size as desired, generally from an average particle size range of about fifty μm to about 800 μm. This may be accomplished by any suitable means such as chopping, pulverizing, milling or grinding the particles. Within the encapsulated surfactant particles, the surfactant itself is from about 2% to about 30% by weight of said encapsulated surfactant, more specifically from about 5% to about 20%.

The coating layer which surrounds the surfactant may also include a solvent. The solvent should be capable of dissolving the polymer. The solvent may be any solvent known for this purpose. For example, if the polymer is polyvinyl acetate, suitable solvents include of ethyl acetate, diethyl ether, acetone, benzene, ethylene dichloride, methanol, methyl ethyl ketone, ethanol, toluene, xylene, amyl acetate, and combinations thereof.

The extrusion and spray coating methods may be combined to provide a desired thickness of coating, and/or to provide a combination of different coating materials. For example, a surfactant may be encapsulated with polyvinyl acetate via the extrusion method with a subsequent coating of gum arabic via a spray coating method.

The coating or encapsulating material may be specifically prepared to have a desired tensile strength, especially where the encapsulated surfactant is included in a gum composition. The advantage of manipulating the tensile strength of the coating is to achieve the desired release rate of the core material, which will include the surfactant. This is desirable because as the surfactant is released into a gum composition from the encapsulating material, flavor is also released from the gum composition. By controlling or extending the release rate of the surfactant, the release rate and amount of the flavor from the gum is also affected and may be desirably extended or increased.

Examples of useful encapsulating materials include cellulose, cellulose derivatives, starches, carbohydrates, gums, polyolefins, polyesters, waxes, vinyl polymers, gelatin, zein and combinations thereof. Specific vinyl polymers include polyethylene, crosslinked polyvinyl pyrrolidone, polymethylmethacrylate, polylactic acid, polyhydroxyalkanoates, ethylcellulose, polyvinyl acetate phthalate, polyethyleneglycol esters, methacrylic acid-co-methylmethacrylate, acrylic polymers and copolymers, carboxyvinyl polymer, polyamides, polystyrene, polyvinyl acetate, other encapsulating materials described herein, and combinations thereof. More specifically, in some embodiments, the encapsulating material may include polyvinyl acetate, gum arabic, and combinations thereof.

Methods of extending the release of flavor from gum compositions are also provided. These methods include the preparation of a gum composition including a gum base, a flavor and a surfactant having HLB of about 7 or higher. In these compositions, the surfactant is optionally encapsulated, which may be effected by either extrusion or a spray coating technique. Several acceptable encapsulating materials are described hereinabove.

Methods of increasing flavor release from a gum composition are also provided which include providing a gum composition comprising a gum base and a flavor and subjecting the gum composition to mastication. Subsequently, a surfactant is added to the gum composition during chewing. The subsequently added surfactant may be in an encapsulated form.

Emulsifiers can include molecules with a glycerol backbone esterified with acetic acid, lactic acid, tartaric acid or citric acid to incorporate fatty acid side chains. In some embodiments, emulsifiers can include distilled monoglycerides, acetic acid esters of mono and diglycerides, citric acid esters of mono and diglycerides, lactic acid esters of mono and diglycerides, mono and diglycerides, polyglycerol esters of fatty acids, ceteareth-20, polyglycerol polyricinoleate, propylene glycol esters of fatty acids, polyglyceryl laurate, glyceryl cocoate, gum arabic, acacia gum, sorbitan monostearates, sorbitan tristearates, sorbitan monolaurate, sorbitan monooleate, sodium stearoyl lactylates, calcium stearoyl lactylates, diacetyl tartaric acid esters of mono- and diglycerides, glyceryl tricaprylate-caprate/medium chain triglycerides, glyceryl dioleate, glyceryl oleate, glyceryl lacto esters of fatty acids, glyceryl lacto palmitate, glyceryl stearate, glyceryl laurate, glycerly dilaurate, glyceryl monoricinoleate, triglyceryl monostearate, hexaglyceryl distearate, decaglyceryl monostearate, decaglyceryl dipalmitate, decaglyceryl monooleate, polyglyceryl 10 hexaoleate, medium chain triglycerides, caprylic/capric triglyceride, propylene glycol monostearate, polysorbate 20, polysorbate 40, polysorbate 60, polysorbate 80, polysorbate 65, hexylglyceryl distearate, triglyceryl monostearate, tweens, spans, stearoyl lactylates, calcium stearoyl-2-lactylate, sodium stearoyl-2-lactylate lecithin, ammonium phosphatide, sucrose esters of fatty acids, sucroglycerides, propane-1,2-diol esters of fatty acids, and combinations thereof.

The features and advantages of encapsulating an emulsifier are more fully shown by the following examples which are provided for purposes of illustration, and are not to be construed as limiting in any way.

TABLE 1
First Set of Chewing Gum Examples with Emulsifiers

| | Percent by weight | | | | | | | |
|---|---|---|---|---|---|---|---|---|
| Component | AAA | BBB | CCC | DDD | EEE | FFF | GGG | HHH |
| Gum base | 30-40 | 30-40 | 25-35 | 25-35 | 22-35 | 30-40 | 30-40 | 25-35 |
| Lecithin | 0.2 | 0 | 0 | 0 | 0 | 0 | 0.2 | 0.2 |
| Bulking Agent | 54-59 | 55-60 | 59-64 | 59-64 | 59-64 | 54-59 | 54-59 | 58-63 |
| ≧7 HLB | 0.04 | 0.1 | 0.3 | 0.5 | 0.7 | 1.0 | 0.04 | 0.04 |
| Surfactant | | | | | | | | |
| Encapsulated ≧ 7 | 0 | 0 | 0 | 0 | 0 | 0 | 0 | 1.0 (2% |
| HLB Surfactant | | | | | | | | surfactant) |
| (based on total weight | | | | | | | | |
| of the surfactant | | | | | | | | |
| and the encapsulating | | | | | | | | |
| materials) | | | | | | | | |
| Flavors | 2.45 | 2.45 | 2.45 | 2.45 | 2.45 | 2.45 | 2.45 | 2.45 |
| Cooling agent | 0.76 | 0.76 | 0.76 | 0.76 | 0.76 | 0.76 | 0.76 | 0.76 |
| Glycerine | 4.0 | 4.0 | 4.0 | 4.0 | 4.0 | 4.0 | 4.0 | 4.0 |
| Intense sweetener | 2.68 | 2.68 | 2.68 | 2.68 | 2.68 | 2.68 | 2.68 | 2.68 |
| (which may include a | | | | | | | | |
| combination of | | | | | | | | |
| encapsulated | | | | | | | | |
| sweeteners and | | | | | | | | |
| non-encapsulated | | | | | | | | |
| sweeteners) | | | | | | | | |

In some embodiments, the gum base in these examples may include 3% to 25% by weight of a filler such as, for example, talc, dicalcium phosphate, and calcium carbonate (the amount of filler in the gum base is based on the weight percent of the gum region composition, for example, in the above compositions A-H, if a gum region composition includes 5% filler, the amount of gum base will be 5% less than the range recited in the table, i.e., from 23-37%).

The compositions for Examples AAA-HHH in the first set of examples with emulsifiers were prepared using the components in this first set of examples with emulsifiers by first combining the gum base and fillers under heat at about 85° C. This combination was then mixed with the bulking agents, lecithin and glycerin for about five minutes. The flavor blends, which include a pre-mix of the flavors, cooling agents and surfactants, were added and mixed for one minute. Finally, intense sweeteners were added and mixed for five minutes.

Each of the compositions AAA-HHH showed an overall increase in the amount of flavor that was released from the gum composition compared to a composition that did not include a surfactant having HLB greater than or equal to 7. In addition, composition HHH, which included an encapsulated surfactant, demonstrated an extended release of flavor.

TABLE 2
Second Set of Chewing Gum Examples with Emulsifiers

| | Percent by weight | | | | | | | |
|---|---|---|---|---|---|---|---|---|
| Component | III | JJJ | KKK | LLL | MMM | NNN | OOO | PPP |
| Gum base | 30-40 | 30-40 | 25-35 | 30 | 30 | 30 | 30 | 25-35 |
| Lecithin | 0.2 | 0.2 | 0.2 | 0 | 0 | 0 | 0 | 0.2 |
| Bulking | 53-58 | 53-58 | 58-63 | 59-64 | 59-64 | 55-65 | 55-65 | 55-65 |
| Agent | | | | | | | | |
| ≧7 HLB | 0.04 | 0.04 | 0.04 | 0.5 | 0.3 | 0.5 | 0.3 | 0 |
| Surfactant | | | | | | | | |
| Encapsulated | 0.5-1 (5% | 0.5-1 | 0.5-1 | 0 | 0 | 0 | 0 | 0.5-1 |
| ≧7 HLB | surfactant) | (10% | (30% | | | | | (10% |
| Surfactant | | surfactant) | surfactant) | | | | | surfactant) |
| (based on the | | | | | | | | |
| total weight | | | | | | | | |
| of the | | | | | | | | |
| surfactant | | | | | | | | |
| and | | | | | | | | |
| encapsulating | | | | | | | | |
| materials) | | | | | | | | |
| Flavors | 2.45 | 2.45 | 2.45 | 2.45 | 2.45 | 2.45 | 2.45 | 2.45 |
| Cooling | 0.76 | 0.76 | 0.76 | 0.76 | 0.76 | 0.76 | 0.76 | 0.76 |
| agent | | | | | | | | |
| Glycerine | 4.0 | 4.0 | 4.0 | 4.0 | 4.0 | 4.0 | 4.0 | 4.0 |
| Intense | 2.68 | 2.68 | 2.68 | 2.68 | 2.68 | 2.68 | 2.68 | 2.68 |
| sweetener | | | | | | | | |
| (which may | | | | | | | | |
| include a | | | | | | | | |
| combination | | | | | | | | |
| of | | | | | | | | |
| encapsulated | | | | | | | | |
| sweeteners | | | | | | | | |
| and non- | | | | | | | | |
| encapsulated | | | | | | | | |
| sweeteners) | | | | | | | | |

In some embodiments, the gum base may include 3% to 25% by weight of a filler such as, for example, talc, dicalcium phosphate, and calcium carbonate (the amount of filler in the gum base is based on the weight percent of the gum region composition, for example, in the above compositions III-PPP, if a gum region composition includes 5% filler, the amount of gum base will be 5% less than the range recited in the table, i.e., from 23-37%).

The compositions for Examples III-PPP were prepared using the components in Table showing the second set of examples with emulsifiers by first combining the gum base and fillers under heat at about 85° C. This combination was then mixed with the bulking agents, lecithin and glycerin for about five minutes. The flavor blends which include a pre-mix of the flavors, cooling agents, and surfactants were added and mixed for one minute. Finally, intense sweeteners were added and mixed for five minutes.

Each of the compositions III-PPPP showed an overall increase in the amount of flavor which was released from the gum composition compared to a composition which did not include a surfactant having HLB greater than or equal to seven. In addition, compositions III, JJJ, KKK, and PPP include an encapsulated surfactant demonstrated an extended release of flavor.

Further information regarding the encapsulation of a surfactant can be found U.S. patent application Ser. No. 11/135,149 entitled “Enhanced Flavor Release Comestible Compositions and Methods for Same” and filed May 23, 2005, the entire contents of which are incorporated herein by reference for all purposes.

### Ingredients—Flavor Potentiators

In some embodiments, the release profiles of one or more flavor potentiators can be managed. Flavor potentiators can consist of materials that may intensify, supplement, modify or enhance the taste and/or aroma perception of an original material without introducing a characteristic taste and/or aroma perception of their own. In some embodiments, potentiators designed to intensity, supplement, modify, or enhance the perception of flavor, sweetness, tartness, umami, kokumi, saltiness and combinations thereof can be included. In some embodiments, monoammonium glycyrrhizinate, licorice glycyrrhizinates, citrus aurantium, maltol, ethyl maltol, vanilla, vanillin, ethyl vanillin, and combinations thereof may be included. In some embodiments, sugar acids, sodium chloride, potassium chloride, sodium acid sulfate, and combinations thereof may be included. In other examples, glutamates such as monosodium glutamate (MSG), monopotassium glutamate, hydrolyzed vegetable protein, hydrolyzed animal protein, yeast extract, and combinations thereof are included. Further examples can include adenosine monophosphate (AMP), glutathione, and nucleotides such as inosine monophosphate (IMP), disodium inosinate, xanthosine monophosphate, guanylate monophosphate (GMP), and combinations thereof. Further examples of flavor potentiator compositions that impart kokumi are also included in U.S. Pat. No. 5,679,397 to Kuroda et al., the entire contents of which are incorporated in its entirety herein by reference.

Illustrations of the encapsulation of flavor potentiators can be found in examples 1, 50, 11, 60, 10, 59, 9, 58, 102, 108, 113, 152, 158, and 163 provided herein. Typically, encapsulation of a flavor potentiator will result in a delay in the release of the predominant amount of the flavor potentiator during consumption of an edible composition that includes the encapsulated flavor potentiator (e.g., as part of a delivery system added as an ingredient to the edible composition). In some embodiments, the release profile of the ingredient (e.g., the flavor potentiator) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a flavor potentiator include the intensity, duration, and type of mixing, the order of addition of ingredients to the mixing process, the positioning or location of ingredients in the final edible composition (e.g., whether or not layers or coating of ingredients are created), etc.

### Ingredients—Flavors

In some embodiments, the release profiles of one or more flavorants can be managed. In some embodiments, flavorants may include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, paw paw, apricot, banana, melon, apricot, ume, cherry, raspberry, blackberry, tropical fruit, mango, mangosteen, pomegranate, papaya and so forth. Other potential flavors whose release profiles can be managed include a milk flavor, a butter flavor, a cheese flavor, a cream flavor, and a yoghurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, an onion flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below.

In some embodiments, other flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, .e., melonal (melon), 2,6-dimethyloctanal (green fruit), and 2-dodecenal (citrus, mandarin), cherry, grape, blueberry, blackberry, strawberry shortcake, and mixtures thereof.

In some embodiments, a flavoring agent may be employed in either liquid form and/or dried form. When employed in the latter form, suitable drying means such as spray drying the oil may be used. Alternatively, the flavoring agent may be absorbed onto water soluble materials, such as cellulose, starch, sugar, maltodextrin, gum arabic and so forth or may be encapsulated. In still other embodiments, the flavoring agent may be adsorbed onto silicas, zeolites, and the like. The actual techniques for preparing such dried forms are well-known.

In some embodiments, the flavoring agents may be used in many distinct physical forms. Without being limited thereto, such physical forms include free forms, such as spray dried, powdered, beaded forms, encapsulated forms, and mixtures thereof.

Illustrations of the encapsulation of flavors can be found in examples 8, 57, 7, and 56 provided herein. Typically, encapsulation of a flavor will result in a delay in the release of the predominant amount of the flavor during consumption of an edible composition that includes the encapsulated flavor (e.g., as part of a delivery system added as an ingredient to the edible composition). In some embodiments, the release profile of the ingredient (e.g., the flavor) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a flavor include the intensity, duration, and type of mixing, the order of addition of ingredients to the mixing process, the positioning or location of ingredients in the final edible composition (e.g., whether or not layers or coating of ingredients are created), etc.

### Ingredients—Food Acids

In some embodiments, the release profiles of one or more acids may be managed. Acids can include, but are not limited to acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof.

Illustrations of the encapsulation of a food acid can be found in examples 4, 53, 5, 54, 6, 55, 104, 105, 106, 107, 154, 155, 156, and 157 provided herein. Typically, encapsulation of a food acid will result in a delay in the release of the predominant amount of the active during consumption of an edible composition that includes the encapsulated food acid (e.g., as part of a delivery system added as an ingredient to the edible composition). In some embodiments, the release profile of the ingredient (e.g., the food acid) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a food acid include the intensity, duration, and type of mixing, the order of addition of ingredients to the mixing process, the positioning or location of ingredients in the final edible composition (e.g., whether or not layers or coating of ingredients are created), etc.

### Ingredients—Sweeteners

In some embodiments, the release profiles of one or more sweeteners may be managed. The sweeteners involved may be selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Without being limited to particular sweeteners, representative categories and examples include:
(a) water-soluble sweetening agents such as dihydrochalcones, monellin, monatin, steviosides, glycyrrhizin, dihydroflavenol, and sugar alcohols such as sorbitol, mannitol, maltitol, and L-aminodicarboxylic acid aminoalkenoic acid ester amides, such as those disclosed in U.S. Pat. No. 4,619,834, which disclosure is incorporated herein by reference, and mixtures thereof;
(b) water-soluble artificial sweeteners such as soluble saccharin salts, i.e., sodium or calcium saccharin salts, cyclamate salts, acesulfame salts (including aspartame-acesulfame salt known by the trade name Twinsweet™ from Holland Sweetener Company, Geleen the Netherlands), such as the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;
(c) dipeptide based sweeteners, such as L-aspartic acid derived sweeteners, such as L-aspartyl-L-phenylalanine methyl ester (Aspartame) and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, neotame, and mixtures thereof;
(d) water-soluble sweeteners derived from naturally occurring water-soluble sweeteners, such as steviosides, chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro4,6,1′,6′-tetradeoxygalacto-sucrose; and 4,6,1′,6′-tetradeoxy-sucrose, and mixtures thereof;
(e) protein based sweeteners such as thaumaoccous danielli (Thaumatin I and II), talin, and (f) amino acid based sweeteners.

The intense sweetening agents may be used in many distinct physical forms well-known in the art to provide an initial burst of sweetness and/or a prolonged sensation of sweetness. Without being limited thereto, such physical forms include free forms, such as spray dried, powdered, beaded forms, encapsulated forms, and mixtures thereof. In one embodiment, the sweetener is a high intensity sweetener such as aspartame, sucralose, and acesulfame potassium (e.g., ace-K).

In some embodiments, the sweetener may be a polyol. Polyols can include, but are not limited to glycerol, sorbitol, malititol, maltitol syrup, mannitol, isomalt, erythritol, xylitol, hydrogenated starch hydrolysates, polyglycitol syrup, polyglycitol powder, lactitol, and combinations thereof.

The active component (e.g., sweetener), which is part of the delivery system, may be used in amounts necessary to impart the desired effect associated with use of the active component (e.g., sweetness).

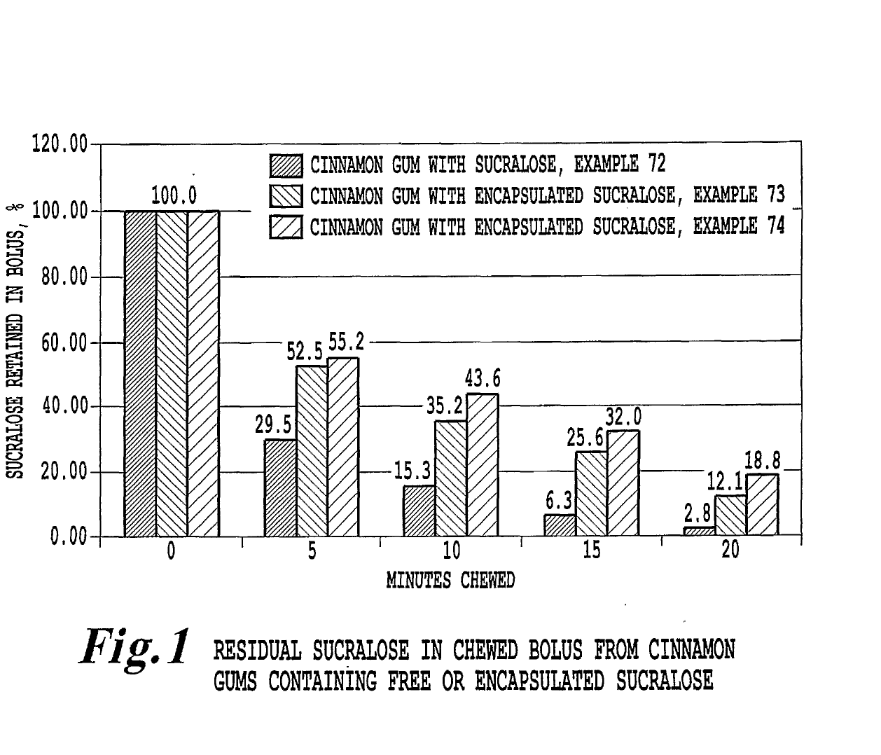

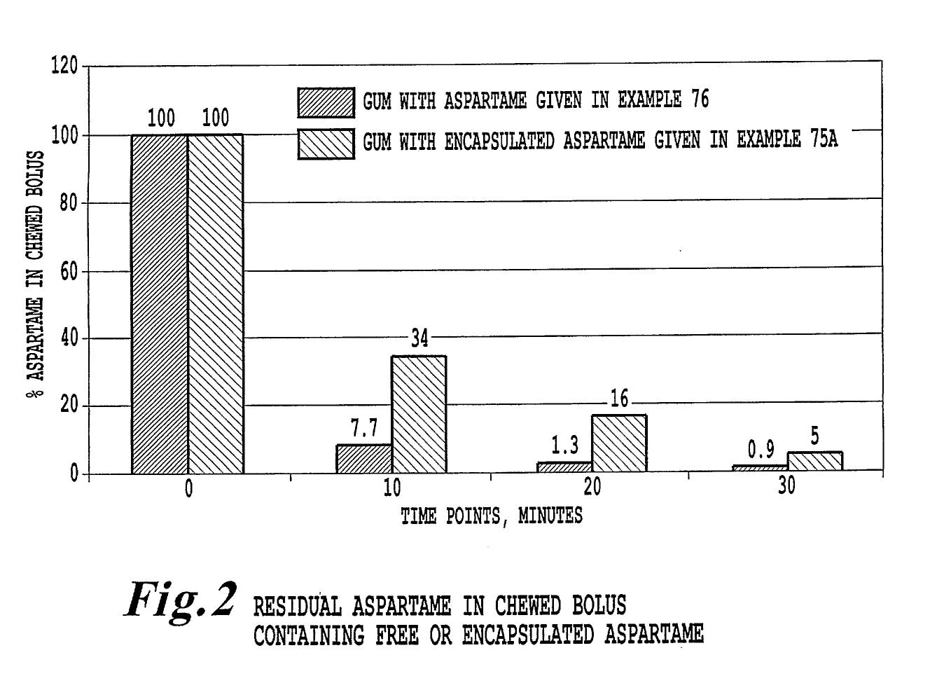

Illustrations of the encapsulation of sweeteners can be found in examples 23, 73, 24, 74, 25A, 25B, 25C, 26, 27, 72, 75A, 75B, 75C, 76, 77, 101, 102, 103, 104, 106 through 114 inclusive, 116 through 119 inclusive, 151, 152, 153, 154, 156 through 164 inclusive, 166, 167, 168, 169, FIG. 1, and FIG. 2 provided herein. Typically, encapsulation of a sweetener will result in a delay in the release of the predominant amount of the active during consumption of an edible composition that includes the encapsulated sweetener (e.g., as part of a delivery system added as an ingredient to the edible composition). In some embodiments, the release profile of the ingredient (e.g., the sweetener) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a sweetener include the intensity, duration, and type of mixing, the order of addition of ingredients to the mixing process, the positioning or location of ingredients in the final edible composition (e.g., whether or not layers or coating of ingredients are created), etc.

### Ingredients—Micronutrients

In some embodiments, the release profiles of one or more micronutrients can be managed. Micronutrients can include materials that have an impact on the nutritional well being of an organism even though the quantity required by the organism to have the desired effect is small relative to macronutrients such as protein, carbohydrate, and fat. Micronutrients can include, but are not limited to vitamins, minerals, enzymes, phytochemicals, antioxidants, and combinations thereof.

In some embodiments, vitamins can include fat soluble vitamins such as vitamin A, vitamin D, vitamin E, and vitamin K and combinations thereof. In some embodiments, vitamins can include water soluble vitamins such as vitamin C (ascorbic acid), the B vitamins (thiamine or B₁, riboflavoin or B₂, niacin or B₃, pyridoxine or B₆, folic acid or B₉, cyanocobalimin or B₁₂, pantothenic acid, biotin), and combinations thereof.

In some embodiments minerals can include but are not limited to sodium, magnesium, chromium, iodine, iron, manganese, calcium, copper, fluoride, potassium, phosphorous, molybdenum, selenium, zinc, and combinations thereof.

In some embodiments micronutrients can include but are not limited to L-carnitine, choline, coenzyme Q10, alpha-lipoic acid, omega-3-fatty acids, pepsin, phytase, trypsin, lipases, proteases, cellulases, and combinations thereof.

Antioxidants can include materials that scavenge free radicals. In some embodiments, antioxidants can include but are not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, and combinations thereof.

In some embodiments phytochemicals can include but are not limited to cartotenoids, chlorophyll, chlorophyllin, fiber, flavanoids, anthocyanins, cyaniding, delphinidin, malvidin, pelargonidin, peonidin, petunidin, flavanols, catechin, epicatechin, epigallocatechin, epigallocatechingallate, theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, soy isoflavones, and combinations thereof.

Illustrations of the encapsulation of a micronutrient can be found in examples 16, 65, 19, 68, 20, 69, 21, 70, 22, 71, 115, 116, 117, 118, 165, 166, 167, and 168 provided herein. Typically, encapsulation of the micronutrient will result in a delay in the release of the predominant amount of the active during consumption of an edible composition that includes the encapsulated micronutrient (e.g., as part of a delivery system added as an ingredient to the edible composition). In some embodiments, the release profile of the ingredient (e.g., the micronutrient) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a micronutrient include the intensity, duration, and type of mixing, the order of addition of ingredients to the mixing process, the positioning or location of ingredients in the final edible composition (e.g., whether or not layers or coating of ingredients are created), etc.

### Ingredients—Sensates

In some embodiments, the release profiles of one or more sensate compounds can be managed. Such sensate compounds can include cooling agents, warming agents, tingling agents, effervescent agents, and combinations thereof. A variety of well known cooling agents may be employed. For example, among the useful cooling agents are included menthol, xylitol, erythritol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, 2-isopropanyl-5-methylcyclohexanol, hydroxycarboxylic acids with 2-6 carbon atoms, cyclohexanamides, menthyl acetate, menthyl lactate, menthyl salicylate, N,2,3-trimethyl-2-isopropyl butanamide (WS-23), N-ethyl-p-menthane-3-carboxamide (WS-3), menthyl succinate, icilin, camphor, bomeol, eucalyptus oil, peppermint oil, methyl salicylate, bornyl acetate, lavender oil, wasabi extracts, horseradish extracts, 3,1-menthoxypropane 1,2-diol, and combinations thereof among others. These and other suitable cooling agents are further described in the following U.S. patents, all of which are incorporated in their entirety by reference hereto: U.S. Pat. Nos. 4,230,688; 4,032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago Perfumary Company Limited, Tokyo, Japan, vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethyleather, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

The sensation of warming or cooling effects may be prolonged with the use of a hydrophobic sweetener as described in U.S. Patent Application Publication 2003/0072842 A1 which is incorporated in its entirety herein by reference. For example, such hydrophobic sweeteners include those of the formulae I-XI referenced therein. Perillartine may also be added as described in U.S. Pat. No. 6,159,509 also incorporated in its entirety herein by reference for all purposes.

In some embodiments, a tingling sensation can be provided. One such tingling sensation is provided by adding jambu, oleoresin, or spilanthol to some examples. In some embodiments, alkylamides extracted from materials such as jambu or sanshool can be included. Additionally, in some embodiments, a sensation is created due to effervescence. Such effervescence is created by combining a basic material with an acidic material. In some embodiments, a basic material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. Examples of “tingling” type sensates can be found in U.S. Pat. No. 6,780,443, the entire contents of which are incorporated herein by reference for all purposes.

Illustrations of the encapsulation of a sensate can be found in examples 12, 61, 143, 62, 14, 63, 103, 109, 110, 111, 153, 159, 160, and 161 provided herein. Typically, encapsulation of the sensate will result in a delay in the release of the predominant amount of the active during consumption of an edible composition that includes the encapsulated sensate (e.g., as part of a delivery system added as an ingredient to the edible composition). In some embodiments, the release profile of the ingredient (e.g., the sensate) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a sensate include the intensity, duration, and type of mixing, the order of addition of ingredients to the mixing process, the positioning or location of ingredients in the final edible composition (e.g., whether or not layers or coating of ingredients are created), etc.

### Ingredients—Mouth Moisteners

In some embodiments, the release profiles of one or more mouth moisteners can be managed. Mouth moisteners can include, but are not limited to, saliva stimulators such as acids and salts and combinations thereof. In some embodiments, acids can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof.

Mouth moisteners can also include hydrocolloid materials that hydrate and may adhere to oral surface to provide a sensation of mouth moistening. Hydrocolloid materials can include naturally occurring materials such as plant exudates, seed gums, and seaweed extracts or they can be chemically modified materials such as cellulose, starch, or natural gum derivatives. In some embodiments, hydrocolloid materials can include pectin, gum arabic, acacia gum, alginates, agar, carageenans, guar gum, xanthan gum, locust bean gum, gelatin, gellan gum, galactomannans, tragacanth gum, karaya gum, curdlan, konjac, chitosan, xyloglucan, beta glucan, furcellaran, gum ghatti, tamarin, bacterial gums, and combinations thereof. Additionally, in some embodiments, modified natural gums such as propylene glycol alginate, carboxymethyl locust bean gum, low methoxyl pectin, and their combinations can be included. In some embodiments, modified celluloses can be included such as microcrystalline cellulose, carboxymethlcellulose (CMC), methylcellulose (MC), hydroxypropylmethylcellulose (HPCM), and hydroxypropylcellulose (MPC), and combinations thereof.

Similarly, humectants which can provide a perception of mouth hydration can be included. Such humectants can include, but are not limited to glycerol, sorbitol, polyethylene glycol, erythritol, and xylitol. Additionally, in some embodiments, fats can provide a perception of mouth moistening. Such fats can include medium chain triglycerides, vegetable oils, fish oils, mineral oils, and combinations thereof.

Illustrations of the encapsulation of a mouth moistening agent can be found in examples 2, 51, 3, 52, 4, 53, 5, 54, 6, 55, 28, 78, 104, 105, 106, 107, 154, 155, 156, and 157 provided herein. Typically, encapsulation of a mouth moistening agent will result in a delay in the release of the predominant amount of the active during consumption of an edible composition that includes the encapsulated mouth moistening agent (e.g., as part of a delivery system added as an ingredient to the edible composition). In some embodiments, the release profile of the ingredient (e.g., the mouth moistening agent) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a mouth moistening agent include the intensity, duration, and type of mixing, the order of addition of ingredients to the mixing process, the positioning or location of ingredients in the final edible composition (e.g., whether or not layers or coating of ingredients are created), etc.

Ingredients—Throat soothing

In some embodiments, the release profiles of one or more ingredients that sooth the throat can be managed. Throat soothing ingredients can include analgesics, anesthetics, demulcents, antiseptic, and combinations thereof. In some embodiments, analgesics/anesthetics can include menthol, phenol, hexylresorcinol, benzocaine, dyclonine hydrochloride, benzyl alcohol, salicyl alcohol, and combinations thereof. In some embodiments, demulcents can include but are not limited to slippery elm bark, pectin, gelatin, and combinations thereof. In some embodiments, antiseptic ingredients can include cetylpyridinium chloride, domiphen bromide, dequalinium chloride, and combinations thereof.

In some embodiments, antitussive ingredients such as chlophedianol hydrochloride, codeine, codeine phosphate, codeine sulfate, dextromethorphan, dextromethorphan hydrobromide, diphenhydramine citrate, and diphenhydramine hydrochloride, and combinations thereof can be included.

In some embodiments, throat soothing agents such as honey, propolis, aloe vera, green and/or red pepper extract, glycerine, menthol and combinations thereof can be included. In still other embodiments, cough suppressants can be included. Such cough suppressants can fall into two groups: those that alter the consistency or production of phlegm such as mucolytics and expectorants; and those that suppress the coughing reflex such as codeine (narcotic cough suppressants), antihistamines, dextromethorphan and isoproterenol (non-narcotic cough suppressants). In some embodiments, ingredients from either or both groups can be included.

In still other embodiments, antitussives can include, but are not limited to, the group consisting of codeine, dextromethorphan, dextrorphan, diphenhydramine, hydrocodone, noscapine, oxycodone, pentoxyverine and combinations thereof. In some embodiments, antihistamines can include, but are not limited to, acrivastine, azatadine, brompheniramine, chlorpheniramine, clemastine, cyproheptadine, dexbrompheniramine, dimenhydrinate, diphenhydramine, doxylamine, hydroxyzine, meclizine, phenindamine, phenyltoloxamine, promethazine, pyrilamine, tripelennamine, triprolidine and combinations thereof. In some embodiments, non-sedating antihistamines can include, but are not limited to, astemizole, cetirizine, ebastine, fexofenadine, loratidine, terfenadine, and combinations thereof.

In some embodiments, expectorants can include, but are not limited to, ammonium chloride, guaifenesin, ipecac fluid extract, potassium iodide and combinations thereof. In some embodiments, mucolytics can include, but are not limited to, acetylcycsteine, ambroxol, bromhexine and combinations thereof. In some embodiments, analgesic, antipyretic and anti-inflammatory agents can include, but are not limited to, acetaminophen, aspirin, diclofenac, diflunisal, etodolac, fenoprofen, flurbiprofen, ibuprofen, ketoprofen, ketorolac, nabumetone, naproxen, piroxicam, caffeine and mixtures thereof. In some embodiments, local anesthetics can include, but are not limited to, lidocaine, benzocaine, phenol, dyclonine, benzonotate and mixtures thereof.

In some embodiments nasal decongestants and ingredients that provide the perception of nasal clearing can be included. In some embodiments, nasal decongestants can include but are not limited to phenylpropanolamine, pseudoephedrine, ephedrine, phenylephrine, oxymetazoline, and combinations thereof. In some embodiments ingredients that provide a perception of nasal clearing can include but are not limited to menthol, camphor, bomeol, ephedrine, eucalyptus oil, peppermint oil, methyl salicylate, bornyl acetate, lavender oil, wasabi extracts, onion extracts, horseradish extracts, and combinations thereof. In some embodiments, a perception of nasal clearing can be provided by odoriferous essential oils, extracts from woods, gums, flowers and other botanicals, resins, animal secretions, and synthetic aromatic materials.

Illustrations of the encapsulation of a throat soothing agent can be found in examples 14, 63, 28, 78, 103, 111, 153, and 161 provided herein. Typically, encapsulation of a throat soothing agent will result in a delay in the release of the predominant amount of the active during consumption of an edible composition that includes the encapsulated throat soothing agent (e.g., as part of a delivery system added as an ingredient to the edible composition). In some embodiments, the release profile of the ingredient (e.g., the dental care active) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a throat soothing agent include the intensity, duration, and type of mixing, the order of addition of ingredients to the mixing process, the positioning or location of ingredients in the final edible composition (e.g., whether or not layers or coating of ingredients are created), etc.

### Ingredients—Colors

In some embodiments, one or more colors can be included. As classified by the United States Food, Drug, and Cosmetic Act (21 C.F.R. 73), colors can include exempt from certification colors (sometimes referred to as natural even though they can be synthetically manufactured) and certified colors (sometimes referred to as artificial), or combinations thereof. In some embodiments, exempt from certification or natural colors can include, but are not limited to annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), β-apo-8′-carotenal (E160e), 0-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and combinations thereof.

In some embodiments, certified colors can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (153), black PN/brilliant black BN (E151), green S/acid brilliant green BS (E142), and combinations thereof. In some embodiments, certified colors can include FD&C aluminum lakes. These consist of the aluminum salts of FD&C dyes extended on an insoluble substrate of alumina hydrate. Additionally, in some embodiments, certified colors can be included as calcium salts.

Typically, encapsulation of a color will result in a delay in the release of the predominant amount of the active during consumption of an edible composition that includes the encapsulated color (e.g., as part of a delivery system added as an ingredient to the edible composition). In some embodiments, the release profile of the ingredient (e.g., the color) can be managed by managing various characteristics of the ingredient, delivery system containing the ingredient, and/or the edible composition containing the delivery system and/or how the delivery system or edible composition is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the edible composition, the distribution or location of the ingredient or the delivery system in the edible composition, distribution of particle sizes of the delivery system, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc. Variables in the mixing process that might change the release profile for a color include the intensity, duration, and type of mixing, the order of addition of ingredients to the mixing process, the positioning or location of ingredients in the final edible composition (e.g., whether or not layers or coating of ingredients are created), etc.

### Multiple Ingredients

In some embodiments, a delivery system or edible composition may include two or more ingredients for which managed release from the edible composition during consumption of the edible composition is desired. In some embodiments, the ingredients may be encapsulated separately in different delivery systems. Alternatively, in some embodiments the ingredients may be encapsulated in the same delivery system. As another possibility, one or more of the ingredients may be free (e.g., unencapsulated) while one or more other ingredients may be encapsulated.

An edible composition such as, for example, a chewing gum, may include a group of ingredients for which managed release of the group during consumption of the edible composition is desired. Groups of two or more ingredients for which managed release from an edible composition during consumption of the edible composition may be desired include, but are not limited to: color and flavor, multiple flavors, multiple colors, cooling agent and flavor, warming agent and flavor, cooling agent and warming agent, cooling agent and high intensity sweetener, warming agent and high intensity sweetener, multiple cooling agents (e.g., WS-3 and WS-23, WS-3 and menthyl succinate), menthol and one or more cooling agents, menthol and one or more warming agents, multiple warming agents, high intensity sweetener(s) and tooth whitening active(s), high intensity sweetener(s) and breath freshening active(s), an ingredient with some bitterness and a bitterness suppressor for the ingredient, multiple high intensity sweeteners (e.g., ace-k and aspartame), multiple tooth whitening actives (e.g., an abrasive ingredient and an antimicrobial ingredient, a peroxide and a nitrate, a warming agent and a polyol, a cooling agent and a polyol, multiple polyols, a warming agent and micronutrient, a cooling agent and a micronutrient, a warming agent and a mouth moistening agent, a cooling agent and a mouth moistening agent, a warming agent and a throat soothing agent, a cooling agent and a throat soothing agent, a warming agent and a food acid, a cooling agent and food acid, a warming agent and an emulsifier/surfactant, a cooling agent and an emulsifier/surfactant, a warming agent and a color, a cooling agent and a color, a warming agent and a flavor potentiator, a cooling agent and a flavor potentiator, a warming agent with sweetness potentiator, a cooling agent with a sweetness potentiator, a warming agent and an appetite suppressant, a cooling agent and an appetite suppressant, a high intensity sweetener and a flavor, a cooling agent and a teeth whitening agent, a warming agent and a teeth whitening agent, a warming agent and breath freshening agent, a cooling agent and a breath freshening agent, a cooling agent and an effervescing system, a warming agent and an effervescing system, a warming agent and an antimicrobial agent, a cooling agent and an antimicrobial agent, multiple anticalculus ingredients, multiple remineralization ingredients, multiple surfactants, remineralization ingredients with demineralization ingredients, acidic ingredients with acid buffering ingredients, anticalculus ingredients with antibacterial ingredients, remineralization ingredients with anticalculus ingredients, anticalculus ingredients with remineralization ingredients with antibacterial ingredients, surfactant ingredients with anticalculus ingredients, surfactant ingredients with antibacterial ingredients, surfactant ingredients with remineralization ingredients, surfactants with anticalculus ingredients with antibacterial ingredients, multiple types of vitamins or minerals, multiple micronutrients, multiple acids, multiple antimicrobial ingredients, multiple breath freshening ingredients, breath freshening ingredients and antimicrobial ingredients, multiple appetite suppressors, acids and bases that react to effervesce, a bitter compound with a high intensity sweetener, a cooling agent and an appetite suppressant, a warming agent and an appetite suppressant, a high intensity sweetener and an appetite suppressant, a high intensity sweetener with an acid, a probiotic ingredient and a prebiotic ingredient, a vitamin and a mineral, a metabolic enhancement ingredient with a macronutrient, a metabolic enhancement ingredient with a micronutrient, an enzyme with a substrate, a high intensity sweetener with a sweetness potentiator, a cooling compound with a cooling potentiator, a flavor with a flavor potentiator, a warming compound with a warming potentiator, a flavor with salt, a high intensity sweetener with salt, an acid with salt, a cooling compound with salt, a warming compound with salt, a flavor with a surfactant, an astringent compound with an ingredient to provide a sensation of hydration, an astringent compound with an ingredient preventing salivary protein precipitation, etc. In some embodiments, the multiple ingredients may be part of the same delivery system or may be part of different delivery systems. Different delivery systems may use the same or different encapsulating materials.

Illustrations of the encapsulation of multiple ingredients can be found in examples 101 through 119 inclusive, 151 through 164 inclusive, 166, 167, 168, 169, 75B, 75C, 76, and 77 provided herein. Typically, encapsulation of the multiple ingredients will result in a delay in the release of the predominant amount of the multiple ingredients during consumption of an edible composition that includes the encapsulated multiple ingredients (e.g., as part of a delivery system added as an ingredient to the edible composition). This may be particularly helpful in situations wherein separate encapsulation of the ingredients may cause them to release with different release profiles. For example, different high intensity sweeteners may have different release profiles because they have different water solubilities or differences in other characteristics. Encapsulating them together may cause them to release more simultaneously.

In some embodiments, the release profile of the multiple ingredients can be managed by managing various characteristics of the multiple ingredients, the delivery system containing the multiple ingredients, and/or the edible composition containing the delivery system and/or how the delivery system or edible composition is made in a manner as previously discussed above.

An additional listing of exemplary ingredients for which managed release from an edible composition may be desired is provided in Table 3 below. Table 3 also provides suitable amounts for the optional ingredients based on chewing gum compositions, which may include a gum region, center-fill region and a coating. The optional ingredients may be used in differing amounts in other types of edible compositions. Table 3 is only representative and is not to be construed to limit the ingredients that can be included in the various edible compositions described herein in any way.

TABLE 3

| Components | Coating | Center-fill | Gum Region |
|---|---|---|---|
| I. Sensates | | | |
| A. Cooling agents | | | |
| Menthol | 10-500 ppm | 10-500 ppm | 500-20,000 ppm |
| Xylitol | 5-80% | 5-95% | 5-80% |
| Erythritol | 5-80% | 5-95% | 5-80% |
| Menthane | 10-500 ppm | 10-500 ppm | 500-20,000 ppm |
| Menthone | 10-500 ppm | 10-500 ppm | 500-20,000 ppm |
| Menthyl acetate | 10-500 ppm | 10-500 ppm | 500-20,000 ppm |
| Menthyl salicylate | 10-500 ppm | 10-500 ppm | 500-20,000 ppm |
| WS-23 | 10-500 ppm | 10-500 ppm | 500-20,000 ppm |
| WS-3 | 10-500 ppm | 10-500 ppm | 500-20,000 ppm |
| Menthyl succinate | 10-500 ppm | 10-500 ppm | 500-20,000 ppm |
| 3,1-menthoxypropane 1,2-diol | 10-500 ppm | 10-500 ppm | 500-20,000 ppm |
| Glutarate esters | 10-500 ppm | 10-500 ppm | 500-20,000 ppm |
| dextrose | 10-500 ppm | 10-500 ppm | 500-20,000 ppm |
| sorbitol | 10-500 ppm | 10-500 ppm | 500-20,000 ppm |
| ketals | 10-500 ppm | 10-500 ppm | 500-20,000 ppm |
| menthone ketals | 10-500 ppm | 10-500 ppm | 500-20,000 ppm |
| menthone glycerol ketals | 10-500 ppm | 10-500 ppm | 500-20,000 ppm |
| substituted p-menthanes | 10-500 ppm | 10-500 ppm | 500-20,000 ppm |
| acyclic carboxamides | 10-500 ppm | 10-500 ppm | 500-20,000 ppm |
| mono menthyl glutarate | 10-500 ppm | 10-500 ppm | 500-20,000 ppm |
| substituted cyclohexanamides | 10-500 ppm | 10-500 ppm | 500-20,000 ppm |
| substituted cyclohexane | 10-500 ppm | 10-500 ppm | 500-20,000 ppm |
| carboxamides | | | |
| substituted ureas and | 10-500 ppm | 10-500 ppm | 500-20,000 ppm |
| sulfonamides | | | |
| substituted menthanols | 10-500 ppm | 10-500 ppm | 500-20,000 ppm |
| hydroxymethyl | 10-500 ppm | 10-500 ppm | 500-20,000 ppm |
| hydroxymethyl derivatives of | 10-500 ppm | 10-500 ppm | 500-20,000 ppm |
| p-menthane | | | |
| 2-mercapto-cyclo-decanone | 10-500 ppm | 10-500 ppm | 500-20,000 ppm |
| hydroxycarboxylic acids with | 10-500 ppm | 10-500 ppm | 500-20,000 ppm |
| 2-6 carbon atoms | | | |
| cyclohexanamides | 10-500 ppm | 10-500 ppm | 500-20,000 ppm |
| l-isopulegol | 10-500 ppm | 10-500 ppm | 500-20,000 ppm |
| 3-(l-menthoxy)-2- | 10-500 ppm | 10-500 ppm | 500-20,000 ppm |
| methylpropane-1,2-diol | | | |
| p-menthane-2,3-diol | 10-500 ppm | 10-500 ppm | 500-20,000 ppm |
| p-menthane-3,8-diol | 10-500 ppm | 10-500 ppm | 500-20,000 ppm |
| 6-isopropyl-9-methyl-1,4- | 10-500 ppm | 10-500 ppm | 500-20,000 ppm |
| dioxaspiro[4,5]decane-2- | | | |
| methanol | | | |
| trimethylcyclohexanol | 10-500 ppm | 10-500 ppm | 500-20,000 ppm |
| N-ethyl-2-isopropyl-5- | 10-500 ppm | 10-500 ppm | 500-20,000 ppm |
| methylcyclohexanecarboxamide | | | |
| Japanese mint oil | 10-500 ppm | 10-500 ppm | 500-20,000 ppm |
| peppermint oil | 10-500 ppm | 10-500 ppm | 500-20,000 ppm |
| 3-(l-menthoxy)ethan-1-ol | 10-500 ppm | 10-500 ppm | 500-20,000 ppm |
| 3-(l-menthoxy)propan-1-ol | 10-500 ppm | 10-500 ppm | 500-20,000 ppm |
| 3-(l-menthoxy)butan-1-ol | 10-500 ppm | 10-500 ppm | 500-20,000 ppm |
| l-menthylacetic acid N- | 10-500 ppm | 10-500 ppm | 500-20,000 ppm |
| ethylamide | | | |
| l-menthyl-4-hydroxypentanoate | 10-500 ppm | 10-500 ppm | 500-20,000 ppm |
| l-menthyl-3-hydroxybutyrate | 10-500 ppm | 10-500 ppm | 500-20,000 ppm |
| N,2,3-trimethyl-2-(1- | 10-500 ppm | 10-500 ppm | 500-20,000 ppm |
| methylethyl)-butanamide | | | |
| n-ethyl-t-2-c-6 nonadienamide | 10-500 ppm | 10-500 ppm | 500-20,000 ppm |
| N,N-dimethyl menthyl | 10-500 ppm | 10-500 ppm | 500-20,000 ppm |
| succinamide | | | |
| substituted p-menthane- | 10-500 ppm | 10-500 ppm | 500-20,000 ppm |
| carboxamides | | | |
| 2-isopropanyl-5- | 10-500 ppm | 10-500 ppm | 500-20,000 ppm |
| methylcyclohexanol | | | |
| menthyl lactate | 10-500 ppm | 10-500 ppm | 500-20,000 ppm |
| WS-30 | 10-500 ppm | 10-500 ppm | 500-20,000 ppm |
| WS-14 | 10-500 ppm | 10-500 ppm | 500-20,000 ppm |
| Eucalyptus extract | 10-500 ppm | 10-500 ppm | 500-20,000 ppm |
| Menthol PG carbonate | 10-500 ppm | 10-500 ppm | 500-20,000 ppm |
| Menthol EG carbonate | 10-500 ppm | 10-500 ppm | 500-20,000 ppm |
| Menthol glyceryl ether | 10-500 ppm | 10-500 ppm | 500-20,000 ppm |
| N-tertbutyl-p-menthane-3- | 10-500 ppm | 10-500 ppm | 500-20,000 ppm |
| carboxamide | | | |
| P-menthane-3-carboxylic acid | 10-500 ppm | 10-500 ppm | 500-20,000 ppm |
| glycerol ester | | | |
| Methyl-2-isopryl-bicyclo | 10-500 ppm | 10-500 ppm | 500-20,000 ppm |
| (2.2.1) | | | |
| Heptane-2-carboxamide | 10-500 ppm | 10-500 ppm | 500-20,000 ppm |
| Menthol methyl ether | 10-500 ppm | 10-500 ppm | 500-20,000 ppm |
| Methyl glutarate | 10-500 ppm | 10-500 ppm | 500-20,000 ppm |
| menthyl pyrrolidone | 10-500 ppm | 10-500 ppm | 500-20,000 ppm |
| carboxylate | | | |
| WS-5 | 10-500 ppm | 10-500 ppm | 500-20,000 ppm |
| WS-15 | 10-500 ppm | 10-500 ppm | 500-20,000 ppm |
| B. Warming agents | | | |
| vanillyl alcohol n-butylether | 1-1000 ppm | 1-1500 ppm | 10-8000 ppm |
| vanillyl alcohol n-propylether | 1-1000 ppm | 1-1500 ppm | 10-8000 ppm |
| vanillyl alcohol isopropylether | 1-1000 ppm | 1-1500 ppm | 10-8000 ppm |
| vanillyl alcohol isobutylether | 1-1000 ppm | 1-1500 ppm | 10-8000 ppm |
| vanillyl alcohol n-aminoether | 1-1000 ppm | 1-1500 ppm | 10-8000 ppm |
| vanillyl alcohol isoamylether | 1-1000 ppm | 1-1500 ppm | 10-8000 ppm |
| vanillyl alcohol n-hexylether | 1-1000 ppm | 1-1500 ppm | 10-8000 ppm |
| vanillyl alcohol methylether | 1-1000 ppm | 1-1500 ppm | 10-8000 ppm |
| vanillyl alcohol ethylether | 1-1000 ppm | 1-1500 ppm | 10-8000 ppm |
| gingerol | 1-1000 ppm | 1-1500 ppm | 10-8000 ppm |
| shogaol | 1-1000 ppm | 1-1500 ppm | 10-8000 ppm |
| paradol | 1-1000 ppm | 1-1500 ppm | 10-8000 ppm |
| zingerone | 1-1000 ppm | 1-1500 ppm | 10-8000 ppm |
| capsaicin | 1-1000 ppm | 1-1500 ppm | 10-8000 ppm |
| dihydrocapsaicin | 1-1000 ppm | 1-1500 ppm | 10-8000 ppm |
| nordihydrocapsaicin | 1-1000 ppm | 1-1500 ppm | 10-8000 ppm |
| homocapsaicin | 1-1000 ppm | 1-1500 ppm | 10-8000 ppm |
| homodihydrocapsaicin | 1-1000 ppm | 1-1500 ppm | 10-8000 ppm |
| ethanol | 1-1000 ppm | 1-1500 ppm | 10-8000 ppm |
| isopropyl alcohol | 1-1000 ppm | 1-1500 ppm | 10-8000 ppm |
| iso-amylalcohol | 1-1000 ppm | 1-1500 ppm | 10-8000 ppm |
| benzyl alcohol | 1-1000 ppm | 1-1500 ppm | 10-8000 ppm |
| glycerine | 1-1000 ppm | 1-1500 ppm | 10-8000 ppm |
| chloroform | 1-1000 ppm | 1-1500 ppm | 10-8000 ppm |
| eugenol | 1-1000 ppm | 1-1500 ppm | 10-8000 ppm |
| cinnamon oil | 1-1000 ppm | 1-1500 ppm | 10-8000 ppm |
| cinnamic aldehyde | 1-1000 ppm | 1-1500 ppm | 10-8000 ppm |
| C. Tingling agents | | | |
| Jambu Oleoresin or para cress | 5-500 ppm | 5-500 ppm | 50-5000 ppm |
| Japanese pepper extract | 5-500 ppm | 5-500 ppm | 50-5000 ppm |
| black pepper extract | 5-500 ppm | 5-500 ppm | 50-5000 ppm |
| Echinacea extract | 5-500 ppm | 5-500 ppm | 50-5000 ppm |
| Northern Prickly Ash extract | 5-500 ppm | 5-500 ppm | 50-5000 ppm |
| red pepper oleoresin | 5-500 ppm | 5-500 ppm | 50-5000 ppm |
| effervescing agents | 5-500 ppm | 5-500 ppm | 50-5000 ppm |
| Spilanthol | 5-500 ppm | 5-500 ppm | 50-5000 ppm |
| Sanshool | 5-500 ppm | 5-500 ppm | 50-5000 ppm |
| II. Flavors | | | |
| spearmint oil | 0.01-10.0% | 0.01-10.0% | 0.5-30.0% |
| cinnamon oil | 0.01-10.0% | 0.01-10.0% | 0.5-30.0% |
| oil of wintergreen | 0.01-10.0% | 0.01-10.0% | 0.5-30.0% |
| peppermint oil | 0.01-10.0% | 0.01-10.0% | 0.5-30.0% |
| clove oil | 0.01-10.0% | 0.01-10.0% | 0.5-30.0% |
| bay oil | 0.01-10.0% | 0.01-10.0% | 0.5-30.0% |
| anise oil | 0.01-10.0% | 0.01-10.0% | 0.5-30.0% |
| eucalyptus oil | 0.01-10.0% | 0.01-10.0% | 0.5-30.0% |
| thyme oil | 0.01-10.0% | 0.01-10.0% | 0.5-30.0% |
| cedar leaf oil | 0.01-10.0% | 0.01-10.0% | 0.5-30.0% |
| oil of nutmeg | 0.01-10.0% | 0.01-10.0% | 0.5-30.0% |
| allspice | 0.01-10.0% | 0.01-10.0% | 0.5-30.0% |
| oil of sage | 0.01-10.0% | 0.01-10.0% | 0.5-30.0% |
| mace | 0.01-10.0% | 0.01-10.0% | 0.5-30.0% |
| oil of bitter almonds | 0.01-10.0% | 0.01-10.0% | 0.5-30.0% |
| cassia oil | 0.01-10.0% | 0.01-10.0% | 0.5-30.0% |
| vanilla | 0.01-10.0% | 0.01-10.0% | 0.5-30.0% |
| lemon | 0.01-10.0% | 0.01-10.0% | 0.5-30.0% |
| orange | 0.01-10.0% | 0.01-10.0% | 0.5-30.0% |
| lime | 0.01-10.0% | 0.01-10.0% | 0.5-30.0% |
| grapefruit | 0.01-10.0% | 0.01-10.0% | 0.5-30.0% |
| apple | 0.01-10.0% | 0.01-10.0% | 0.5-30.0% |
| pear | 0.01-10.0% | 0.01-10.0% | 0.5-30.0% |
| peach | 0.01-10.0% | 0.01-10.0% | 0.5-30.0% |
| grape | 0.01-10.0% | 0.01-10.0% | 0.5-30.0% |
| strawberry | 0.01-10.0% | 0.01-10.0% | 0.5-30.0% |
| raspberry | 0.01-10.0% | 0.01-10.0% | 0.5-30.0% |
| cherry | 0.01-10.0% | 0.01-10.0% | 0.5-30.0% |
| plum | 0.01-10.0% | 0.01-10.0% | 0.5-30.0% |
| pineapple | 0.01-10.0% | 0.01-10.0% | 0.5-30.0% |
| apricot | 0.01-10.0% | 0.01-10.0% | 0.5-30.0% |
| watermelon | 0.01-10.0% | 0.01-10.0% | 0.5-30.0% |
| chocolate | 0.01-10.0% | 0.01-10.0% | 0.5-30.0% |
| cola | 0.01-10.0% | 0.01-10.0% | 0.5-30.0% |
| maple | 0.01-10.0% | 0.01-10.0% | 0.5-30.0% |
| dulce de leche | 0.01-10.0% | 0.01-10.0% | 0.5-30.0% |
| raisin | 0.01-10.0% | 0.01-10.0% | 0.5-30.0% |
| caramel | 0.01-10.0% | 0.01-10.0% | 0.5-30.0% |
| cinnamyl acetate | 0.01-10.0% | 0.01-10.0% | 0.5-30.0% |
| cinnamaldehyde | 0.01-10.0% | 0.01-10.0% | 0.5-30.0% |
| citral diethylacetal | 0.01-10.0% | 0.01-10.0% | 0.5-30.0% |
| dihydrocarvyl acetate | 0.01-10.0% | 0.01-10.0% | 0.5-30.0% |
| eugenyl formate | 0.01-10.0% | 0.01-10.0% | 0.5-30.0% |
| p-methylamisol | 0.01-10.0% | 0.01-10.0% | 0.5-30.0% |
| acetaldehyde | 0.01-10.0% | 0.01-10.0% | 0.5-30.0% |
| benzaldehyde | 0.01-10.0% | 0.01-10.0% | 0.5-30.0% |
| anisic aldehyde | 0.01-10.0% | 0.01-10.0% | 0.5-30.0% |
| cinnamic aldehyde | 0.01-10.0% | 0.01-10.0% | 0.5-30.0% |
| citral | 0.01-10.0% | 0.01-10.0% | 0.5-30.0% |
| neral | 0.01-10.0% | 0.01-10.0% | 0.5-30.0% |
| decanal | 0.01-10.0% | 0.01-10.0% | 0.5-30.0% |
| ethyl vanillin | 0.01-10.0% | 0.01-10.0% | 0.5-30.0% |
| heliotrope | 0.01-10.0% | 0.01-10.0% | 0.5-30.0% |
| vanillin | 0.01-10.0% | 0.01-10.0% | 0.5-30.0% |
| alpha-amyl cinnamaldehyde | 0.01-10.0% | 0.01-10.0% | 0.5-30.0% |
| butyraldehyde | 0.01-10.0% | 0.01-10.0% | 0.5-30.0% |
| valeraldehyde | 0.01-10.0% | 0.01-10.0% | 0.5-30.0% |
| citronellal | 0.01-10.0% | 0.01-10.0% | 0.5-30.0% |
| decanal | 0.01-10.0% | 0.01-10.0% | 0.5-30.0% |
| aldehyde C-8 | 0.01-10.0% | 0.01-10.0% | 0.5-30.0% |
| aldehyde C-9 | 0.01-10.0% | 0.01-10.0% | 0.5-30.0% |
| aldehyde C-12 | 0.01-10.0% | 0.01-10.0% | 0.5-30.0% |
| 2-ethyl butyraldehyde | 0.01-10.0% | 0.01-10.0% | 0.5-30.0% |
| hexenal | 0.01-10.0% | 0.01-10.0% | 0.5-30.0% |
| tolyl aldehyde | 0.01-10.0% | 0.01-10.0% | 0.5-30.0% |
| veratraldehyde | 0.01-10.0% | 0.01-10.0% | 0.5-30.0% |
| 2,6-dimethyl-5-heptenal | 0.01-10.0% | 0.01-10.0% | 0.5-30.0% |
| 2,6-dimethyloctanal | 0.01-10.0% | 0.01-10.0% | 0.5-30.0% |
| 2-dodecenal | 0.01-10.0% | 0.01-10.0% | 0.5-30.0% |
| strawberry shortcake | 0.01-10.0% | 0.01-10.0% | 0.5-30.0% |
| pomegranate | 0.01-10.0% | 0.01-10.0% | 0.5-30.0% |
| beef | 0.01-10.0% | 0.01-10.0% | 0.5-30.0% |
| chicken | 0.01-10.0% | 0.01-10.0% | 0.5-30.0% |
| cheese | 0.01-10.0% | 0.01-10.0% | 0.5-30.0% |
| onion | 0.01-10.0% | 0.01-10.0% | 0.5-30.0% |
| III. Tastes | | | |
| A. Sweeteners | | | |
| sucrose | 5-100% | 5-100% | 5-80% |
| dextrose | 5-100% | 5-100% | 5-80% |
| maltose | 5-100% | 5-100% | 5-80% |
| dextrin | 5-100% | 5-100% | 5-80% |
| xylose | 5-100% | 5-100% | 5-80% |
| ribose | 5-100% | 5-100% | 5-80% |
| glucose | 5-100% | 5-100% | 5-80% |
| mannose | 5-100% | 5-100% | 5-80% |
| galactose | 5-100% | 5-100% | 5-80% |
| fructose | 5-100% | 5-100% | 5-80% |
| invert sugar | 5-100% | 5-100% | 5-80% |
| fructo oligo saccharide syrups | 5-100% | 5-100% | 5-80% |
| partially hydrolyzed starch | 5-100% | 5-100% | 5-80% |
| corn syrup solids | 5-100% | 5-100% | 5-80% |
| sorbitol | 5-100% | 5-100% | 5-80% |
| xylitol | 5-100% | 5-100% | 5-80% |
| mannitol | 5-100% | 5-100% | 5-80% |
| galactitol | 5-100% | 5-100% | 5-80% |
| maltitol | 5-100% | 5-100% | 5-80% |
| Isomalt | 5-100% | 5-100% | 5-80% |
| lactitol | 5-100% | 5-100% | 5-80% |
| erythritol | 5-100% | 5-100% | 5-80% |
| hydrogenated starch | 5-100% | 5-100% | 5-80% |
| hydrolysate | | | |
| stevia | 10-20,000 ppm | 10-20,000 ppm | 10-20,000 ppm |
| dihydrochalcones | 10-20,000 ppm | 10-20,000 ppm | 10-20,000 ppm |
| monellin | 10-20,000 ppm | 10-20,000 ppm | 10-20,000 ppm |
| steviosides | 10-20,000 ppm | 10-20,000 ppm | 10-20,000 ppm |
| glycyrrhizin | 10-20,000 ppm | 10-20,000 ppm | 10-20,000 ppm |
| dihydroflavenol | 10-20,000 ppm | 10-20,000 ppm | 10-20,000 ppm |
| L-aminodicarboxylic acid | 10-20,000 ppm | 10-20,000 ppm | 10-20,000 ppm |
| aminoalkenoic acid ester | | | |
| amides | | | |
| sodium or calcium saccharin | 10-20,000 ppm | 10-20,000 ppm | 10-20,000 ppm |
| salts | | | |
| cyclamate salts | 10-20,000 ppm | 10-20,000 ppm | 10-20,000 ppm |
| sodium, ammonium or calcium | 10-20,000 ppm | 10-20,000 ppm | 10-20,000 ppm |
| salt of 3,4-dihydro-6-methyl- | | | |
| 1,2,3-oxathiazine-4-one-2,2- | | | |
| dioxide | | | |
| Acesulfame-K | 10-20,000 ppm | 10-20,000 ppm | 10-20,000 ppm |
| free acid form of saccharin | 10-20,000 ppm | 10-20,000 ppm | 10-20,000 ppm |
| Aspartame | 10-20,000 ppm | 10-20,000 ppm | 10-20,000 ppm |
| Alitame | 10-20,000 ppm | 10-20,000 ppm | 10-20,000 ppm |
| Neotame | 10-20,000 ppm | 10-20,000 ppm | 10-20,000 ppm |
| methyl esters of L-aspartyl-L- | 10-20,000 ppm | 10-20,000 ppm | 10-20,000 ppm |
| phenylglycerine and L-aspartyl- | | | |
| L-2,5-dihydrophenyl-glycine | | | |
| L-aspartyl-2,5-dihydro-L- | 10-20,000 ppm | 10-20,000 ppm | 10-20,000 ppm |
| phenylalanine | | | |
| L-aspartyl-L-(1-cyclohexen)- | 10-20,000 ppm | 10-20,000 ppm | 10-20,000 ppm |
| alanine | | | |
| Sucralose | 10-20,000 ppm | 10-20,000 ppm | 10-20,000 ppm |
| 1-chloro-1′-deoxysucrose | 10-20,000 ppm | 10-20,000 ppm | 10-20,000 ppm |
| 4-chloro-4-deoxy-alpha-D- | 10-20,000 ppm | 10-20,000 ppm | 10-20,000 ppm |
| galactopyranosyl-alpha-D- | | | |
| fructofuranoside | | | |
| 4-chloro-4-deoxygalactosucrose | 10-20,000 ppm | 10-20,000 ppm | 10-20,000 ppm |
| 4-chloro-4-deoxy-alpha-D- | 10-20,000 ppm | 10-20,000 ppm | 10-20,000 ppm |
| galactopyranosyl-1-chloro-1- | | | |
| deoxy-beta-D-fructo-furanoside | | | |
| 4,1′-dichloro-4,1′- | 10-20,000 ppm | 10-20,000 ppm | 10-20,000 ppm |
| dideoxygalactosucrose | | | |
| 1′,6′-dichloro1′,6′- | 10-20,000 ppm | 10-20,000 ppm | 10-20,000 ppm |
| dideoxysucrose | | | |
| 4-chloro-4-deoxy-alpha-D- | 10-20,000 ppm | 10-20,000 ppm | 10-20,000 ppm |
| galactopyranosyl-1,6-dichloro- | | | |
| 1,6-dideoxy-beta-D- | | | |
| fructofuranoside | | | |
| 4,1′,6′-trichloro-4,1′,6′- | 10-20,000 ppm | 10-20,000 ppm | 10-20,000 ppm |
| trideoxygalactosucrose | | | |
| 4,6-dichloro-4,6-dideoxy-alpha- | 10-20,000 ppm | 10-20,000 ppm | 10-20,000 ppm |
| D-galactopyranosyl-6-chloro-6- | | | |
| deoxy-beta-D-fructofuranoside | | | |
| 4,6,6′-trichloro-4,6,6′- | 10-20,000 ppm | 10-20,000 ppm | 10-20,000 ppm |
| trideoxygalactosucrose | | | |
| 6,1′,6′-trichloro-6,1′,6′- | 10-20,000 ppm | 10-20,000 ppm | 10-20,000 ppm |
| trideoxysucrose | | | |
| 4,6-dichloro-4,6-dideoxy-alpha- | 10-20,000 ppm | 10-20,000 ppm | 10-20,000 ppm |
| D-galacto-pyranosyl-1,6- | | | |
| dichloro-1,6-dideoxy-beta-D- | | | |
| fructofuranoside | | | |
| 4,6,1′,6′-tetrachloro4,6,1′,6′- | 10-20,000 ppm | 10-20,000 ppm | 10-20,000 ppm |
| tetradeoxygalacto-sucrose | | | |
| 4,6,1′,6′-tetradeoxy-sucrose | 10-20,000 ppm | 10-20,000 ppm | 10-20,000 ppm |
| Thaumatin I and II | 10-20,000 ppm | 10-20,000 ppm | 10-20,000 ppm |
| Monatin | 10-20,000 ppm | 10-20,000 ppm | 10-20,000 ppm |
| B. Sour | | | |
| acetic acid | 0.00005-10% | 0.00005-10% | 0.00005-10% |
| adipic acid | 0.00005-10% | 0.00005-10% | 0.00005-10% |
| ascorbic acid | 0.00005-10% | 0.00005-10% | 0.00005-10% |
| butyric acid | 0.00005-10% | 0.00005-10% | 0.00005-10% |
| citric acid | 0.00005-10% | 0.00005-10% | 0.00005-10% |
| formic acid | 0.00005-10% | 0.00005-10% | 0.00005-10% |
| fumaric acid | 0.00005-10% | 0.00005-10% | 0.00005-10% |
| glyconic acid | 0.00005-10% | 0.00005-10% | 0.00005-10% |
| lactic acid | 0.00005-10% | 0.00005-10% | 0.00005-10% |
| phosphoric acid | 0.00005-10% | 0.00005-10% | 0.00005-10% |
| malic acid | 0.00005-10% | 0.00005-10% | 0.00005-10% |
| oxalic acid | 0.00005-10% | 0.00005-10% | 0.00005-10% |
| succinic acid | 0.00005-10% | 0.00005-10% | 0.00005-10% |
| tartaric acid | 0.00005-10% | 0.00005-10% | 0.00005-10% |
| C. Bitter/Astringent | | | |
| quinine | 0.01-100 ppm | 0.01-100 ppm | 0.01-100 ppm |
| naringin | 0.01-100 ppm | 0.01-100 ppm | 0.01-100 ppm |
| quassia | 0.01-100 ppm | 0.01-100 ppm | 0.01-100 ppm |
| phenyl thiocarbamide (PTC) | 0.01-100 ppm | 0.01-100 ppm | 0.01-100 ppm |
| 6-n-propylthiouracil (Prop) | 0.01-100 ppm | 0.01-100 ppm | 0.01-100 ppm |
| alum | 0.01-100 ppm | 0.01-100 ppm | 0.01-100 ppm |
| salicin | 0.01-100 ppm | 0.01-100 ppm | 0.01-100 ppm |
| caffeine | 0.01-100 ppm | 0.01-100 ppm | 0.01-100 ppm |
| D. Salty | | | |
| sodium chloride | 0.01-1% | 0.01-1% | 0.01-1% |
| calcium chloride | 0.01-1% | 0.01-1% | 0.01-1% |
| potassium chloride | 0.01-1% | 0.01-1% | 0.01-1% |
| l-lysine | 0.01-1% | 0.01-1% | 0.01-1% |
| IV. Functional agents | | | |
| A. Surfactants | | | |
| salts of fatty acids selected from | 0.001-2% | 0.001-2% | 0.001-2% |
| the group consisting of C₈-C₂₄ | | | |
| palmitoleic acid | 0.001-2% | 0.001-2% | 0.001-2% |
| oleic acid | 0.001-2% | 0.001-2% | 0.001-2% |
| eleosteric acid | 0.001-2% | 0.001-2% | 0.001-2% |
| butyric acid | 0.001-2% | 0.001-2% | 0.001-2% |
| caproic acid | 0.001-2% | 0.001-2% | 0.001-2% |
| caprylic acid | 0.001-2% | 0.001-2% | 0.001-2% |
| capric acid | 0.001-2% | 0.001-2% | 0.001-2% |
| lauric acid | 0.001-2% | 0.001-2% | 0.001-2% |
| myristic acid | 0.001-2% | 0.001-2% | 0.001-2% |
| palmitic acid | 0.001-2% | 0.001-2% | 0.001-2% |
| stearic acid | 0.001-2% | 0.001-2% | 0.001-2% |
| ricinoleic acid | 0.001-2% | 0.001-2% | 0.001-2% |
| arachidic acid | 0.001-2% | 0.001-2% | 0.001-2% |
| behenic acid | 0.001-2% | 0.001-2% | 0.001-2% |
| lignoceric acid | 0.001-2% | 0.001-2% | 0.001-2% |
| cerotic acid | 0.001-2% | 0.001-2% | 0.001-2% |
| sulfated butyl oleate | 0.001-2% | 0.001-2% | 0.001-2% |
| medium and long chain fatty | 0.001-2% | 0.001-2% | 0.001-2% |
| acid esters | | | |
| sodium oleate | 0.001-2% | 0.001-2% | 0.001-2% |
| salts of fumaric acid | 0.001-2% | 0.001-2% | 0.001-2% |
| potassium glomate | 0.001-2% | 0.001-2% | 0.001-2% |
| organic acid esters of mono- | 0.001-2% | 0.001-2% | 0.001-2% |
| and diglycerides | | | |
| stearyl monoglyceridyl citrate | 0.001-2% | 0.001-2% | 0.001-2% |
| succistearin | 0.001-2% | 0.001-2% | 0.001-2% |
| dioctyl sodium sulfosuccinate | 0.001-2% | 0.001-2% | 0.001-2% |
| glycerol tristearate | 0.001-2% | 0.001-2% | 0.001-2% |
| lecithin | 0.001-2% | 0.001-2% | 0.001-2% |
| hydroxylated lecithin | 0.001-2% | 0.001-2% | 0.001-2% |
| sodium lauryl sulfate | 0.001-2% | 0.001-2% | 0.001-2% |
| acetylated monoglycerides | 0.001-2% | 0.001-2% | 0.001-2% |
| succinylated monoglycerides | 0.001-2% | 0.001-2% | 0.001-2% |
| monoglyceride citrate | 0.001-2% | 0.001-2% | 0.001-2% |
| ethoxylated mono- and | 0.001-2% | 0.001-2% | 0.001-2% |
| diglycerides | | | |
| sorbitan monostearate | 0.001-2% | 0.001-2% | 0.001-2% |
| calcium stearyl-2-lactylate | 0.001-2% | 0.001-2% | 0.001-2% |
| sodium stearyl lactylate | 0.001-2% | 0.001-2% | 0.001-2% |
| lactylated fatty acid esters of | 0.001-2% | 0.001-2% | 0.001-2% |
| glycerol and propylene glycerol | | | |
| glycerol-lactoesters of C8-C24 | 0.001-2% | 0.001-2% | 0.001-2% |
| fatty acids | | | |
| polyglycerol esters of C8-C24 | 0.001-2% | 0.001-2% | 0.001-2% |
| fatty acids | | | |
| propylene glycol alginate | 0.001-2% | 0.001-2% | 0.001-2% |
| sucrose C8-C24 fatty acid esters | 0.001-2% | 0.001-2% | 0.001-2% |
| diacetyl tartaric and citric acid | 0.001-2% | 0.001-2% | 0.001-2% |
| esters of mono- and | | | |
| diglycerides | | | |
| triacetin | 0.001-2% | 0.001-2% | 0.001-2% |
| sarcosinate surfactants | 0.001-2% | 0.001-2% | 0.001-2% |
| isethionate surfactants | 0.001-2% | 0.001-2% | 0.001-2% |
| tautate surfactants | 0.001-2% | 0.001-2% | 0.001-2% |
| pluronics | 0.001-2% | 0.001-2% | 0.001-2% |
| polyethylene oxide condensates | 0.001-2% | 0.001-2% | 0.001-2% |
| of alkyl phenols | | | |
| products derived from the | 0.001-2% | 0.001-2% | 0.001-2% |
| condensation of ethylene oxide | | | |
| with the reaction product of | | | |
| propylene oxide and ethylene | | | |
| diamine | | | |
| ethylene oxide condensates of | 0.001-2% | 0.001-2% | 0.001-2% |
| aliphatic alcohols | | | |
| long chain tertiary amine oxides | 0.001-2% | 0.001-2% | 0.001-2% |
| long chain tertiary phosphine | 0.001-2% | 0.001-2% | 0.001-2% |
| oxides | | | |
| long chain dialkyl sulfoxides | 0.001-2% | 0.001-2% | 0.001-2% |
| B. Breath freshening agents | | | |
| spearmint oil | 0.001-10% | 0.001-10% | 0.001-10% |
| peppermint oil | 0.001-10% | 0.001-10% | 0.001-10% |
| wintergreen oil | 0.001-10% | 0.001-10% | 0.001-10% |
| sassafras oil | 0.001-10% | 0.001-10% | 0.001-10% |
| chlorophyll oil | 0.001-10% | 0.001-10% | 0.001-10% |
| citral oil | 0.001-10% | 0.001-10% | 0.001-10% |
| geraniol oil | 0.001-10% | 0.001-10% | 0.001-10% |
| cardamom oil | 0.001-10% | 0.001-10% | 0.001-10% |
| clove oil | 0.001-10% | 0.001-10% | 0.001-10% |
| sage oil | 0.001-10% | 0.001-10% | 0.001-10% |
| carvacrol oil | 0.001-10% | 0.001-10% | 0.001-10% |
| eucalyptus oil | 0.001-10% | 0.001-10% | 0.001-10% |
| cardamom oil | 0.001-10% | 0.001-10% | 0.001-10% |
| magnolia bark extract oil | 0.001-10% | 0.001-10% | 0.001-10% |
| marjoram oil | 0.001-10% | 0.001-10% | 0.001-10% |
| cinnamon oil | 0.001-10% | 0.001-10% | 0.001-10% |
| lemon oil | 0.001-10% | 0.001-10% | 0.001-10% |
| lime oil | 0.001-10% | 0.001-10% | 0.001-10% |
| grapefruit oil | 0.001-10% | 0.001-10% | 0.001-10% |
| orange oil | 0.001-10% | 0.001-10% | 0.001-10% |
| cinnamic aldehyde | 0.001-10% | 0.001-10% | 0.001-10% |
| salicylaldehyde | 0.001-10% | 0.001-10% | 0.001-10% |
| menthol | 0.001-10% | 0.001-10% | 0.001-10% |
| carvone | 0.001-10% | 0.001-10% | 0.001-10% |
| iso-garrigol | 0.001-10% | 0.001-10% | 0.001-10% |
| anethole | 0.001-10% | 0.001-10% | 0.001-10% |
| zinc citrate | 0.01-25% | 0.01-25% | 0.1-15% |
| zinc acetate | 0.01-25% | 0.01-25% | 0.1-15% |
| zinc fluoride | 0.01-25% | 0.01-25% | 0.1-15% |
| zinc ammonium sulfate | 0.01-25% | 0.01-25% | 0.1-15% |
| zinc bromide | 0.01-25% | 0.01-25% | 0.1-15% |
| zinc iodide | 0.01-25% | 0.01-25% | 0.1-15% |
| zinc chloride | 0.01-25% | 0.01-25% | 0.1-15% |
| zinc nitrate | 0.01-25% | 0.01-25% | 0.1-15% |
| zinc flurosilicate | 0.01-25% | 0.01-25% | 0.1-15% |
| zinc gluconate | 0.01-25% | 0.01-25% | 0.1-15% |
| zinc tartarate | 0.01-25% | 0.01-25% | 0.1-15% |
| zinc succinate | 0.01-25% | 0.01-25% | 0.1-15% |
| zinc formate | 0.01-25% | 0.01-25% | 0.1-15% |
| zinc chromate | 0.01-25% | 0.01-25% | 0.1-15% |
| zinc phenol sulfonate | 0.01-25% | 0.01-25% | 0.1-15% |
| zinc dithionate | 0.01-25% | 0.01-25% | 0.1-15% |
| zinc sulfate | 0.01-25% | 0.01-25% | 0.1-15% |
| silver nitrate | 0.01-25% | 0.01-25% | 0.1-15% |
| zinc salicylate | 0.01-25% | 0.01-25% | 0.1-15% |
| zinc glycerophosphate | 0.01-25% | 0.01-25% | 0.1-15% |
| copper nitrate | 0.01-25% | 0.01-25% | 0.1-15% |
| chlorophyll | 0.01-25% | 0.01-25% | 0.1-15% |
| copper chlorophyll | 0.01-25% | 0.01-25% | 0.1-15% |
| chlorophyllin | 0.01-25% | 0.01-25% | 0.1-15% |
| hydrogenated cottonseed oil | 0.5-5% | 0.5-70% | 0.5-15% |
| chlorine dioxide | 0.025-0.50% | 0.025-0.50% | 0.025-0.50% |
| beta cyclodextrin | 0.1-5% | 0.1-5% | 0.1-5% |
| zeolite | 0.1-5% | 0.1-5% | 0.1-5% |
| silica-based materials | 0.1-5% | 0.1-5% | 0.1-5% |
| carbon-based materials | 0.1-5% | 0.1-5% | 0.1-5% |
| enzymes such as laccase, | 0.1-5% | 0.1-5% | 0.1-5% |
| papain, krillase, amylase, | | | |
| glucose oxidase | | | |
| C. Anti-microbial agents | | | |
| cetylpyridinium chloride | 0.01-1% | 0.01-1% | 0.01-1% |
| zinc compounds | 0.01-25% | 0.01-25% | 0.1-15% |
| copper compounds | 0.01-25% | 0.01-25% | 0.1-15% |
| D. Antibacterial agents | | | |
| chlorhexidine | 0.0025-2% | 0.0025-2% | 0.0025-2% |
| alexidine | 0.0025-2% | 0.0025-2% | 0.0025-2% |
| quaternary ammonium salts | 0.0025-2% | 0.0025-2% | 0.0025-2% |
| benzethonium chloride | 0.0025-2% | 0.0025-2% | 0.0025-2% |
| cetyl pyridinium chloride | 0.0025-2% | 0.0025-2% | 0.0025-2% |
| 2,4,4′-trichloro-2′-hydroxy- | 0.0025-2% | 0.0025-2% | 0.0025-2% |
| diphenyl ether (triclosan) | | | |
| E. Anti-calculus agents | | | |
| pyrophosphates | 1-6% | 1-6% | 1-6% |
| triphosphates | 0.1-10% | 0.1-10% | 0.1-10% |
| polyphosphates | 0.1-10% | 0.1-10% | 0.1-10% |
| polyphosphonates | 0.1-10% | 0.1-10% | 0.1-10% |
| dialkali metal pyrophosphate | 1-6% | 1-6% | 1-6% |
| salt | | | |
| tetra alkali polyphosphate salt | 0.1-10% | 0.1-10% | 0.1-10% |
| tetrasodium pyrophosphate | 1-6% | 1-6% | 1-6% |
| tetrapotassium pyrophosphate | 1-6% | 1-6% | 1-6% |
| sodium tripolyphosphate | 0.1-10% | 0.1-10% | 0.1-10% |
| F. Anti-plaque agents | | | |
| chlorhexidine | 0.0025-2% | 0.0025-2% | 0.0025-2% |
| triclosan | 0.01-2% | 0.01-2% | 0.01-2% |
| hexetidine | 0.01-2% | 0.01-2% | 0.01-2% |
| zinc citrate | 0.01-25% | 0.01-25% | 0.1-15% |
| essential oils | 0.001-10% | 0.001-10% | 0.001-10% |
| sodium lauryl sulfate | 0.001-2% | 0.001-2% | 0.001-2% |
| G. Fluoride compounds | | | |
| sodium fluoride | 0.01-1% | 0.01-1% | 0.01-1% |
| sodium monofluorophosphate | 0.01-1% | 0.01-1% | 0.01-1% |
| stannous fluoride | 0.01-1% | 0.01-1% | 0.01-1% |
| H. Quaternary ammonium | | | |
| compounds | | | |
| Benzalkonium Chloride | 0.01-1% | 0.01-1% | 0.01-1% |
| Benzethonium Chloride | 0.01-1% | 0.01-1% | 0.01-1% |
| Cetalkonium Chloride | 0.01-1% | 0.01-1% | 0.01-1% |
| Cetrimide | 0.01-1% | 0.01-1% | 0.01-1% |
| Cetrimonium Bromide | 0.01-1% | 0.01-1% | 0.01-1% |
| Cetylpyridinium Chloride | 0.01-1% | 0.01-1% | 0.01-1% |
| Glycidyl Trimethyl Ammonium | 0.01-1% | 0.01-1% | 0.01-1% |
| Chloride | | | |
| Stearalkonium Chloride | 0.01-1% | 0.01-1% | 0.01-1% |
| I. Remineralization agents | | | |
| phosphopeptide-amorphous | 0.1-5% | 0.1-5% | 0.1-5% |
| calcium phosphate | | | |
| casein phosphoprotein-calcium | 0.1-5% | 0.1-5% | 0.1-5% |
| phosphate complex | | | |
| casein phosphopeptide- | 0.1-5% | 0.1-5% | 0.1-5% |
| stabilized calcium phosphate | | | |
| J. Pharmaceutical actives | | | |
| drugs or medicaments | 0.0001-10% | 0.0001-10% | 0.0001-10% |
| vitamins and other dietary | 0.0001-10% | 0.0001-10% | 0.0001-10% |
| supplements | | | |
| minerals | 0.0001-10% | 0.0001-10% | 0.0001-10% |
| caffeine | 0.0001-10% | 0.0001-10% | 0.0001-10% |
| nicotine | 0.0001-10% | 0.0001-10% | 0.0001-10% |
| fruit juices | 2-10% | 2-60% | 1-15% |
| K. Micronutrients | | | |
| vitamin A | 0.0001-10% | 0.0001-10% | 0.0001-10% |
| vitamin D | 0.0001-10% | 0.0001-10% | 0.0001-10% |
| vitamin E | 0.0001-10% | 0.0001-10% | 0.0001-10% |
| vitamin K | 0.0001-10% | 0.0001-10% | 0.0001-10% |
| vitamin C (ascorbic acid) | 0.0001-10% | 0.0001-10% | 0.0001-10% |
| B vitamins (thiamine or B1, | 0.0001-10% | 0.0001-10% | 0.0001-10% |
| riboflavoin or B2, niacin or B3, | | | |
| pyridoxine or B6, folic acid or | | | |
| B9, cyanocobalimin or B12, | | | |
| pantothenic acid, biotin) | | | |
| sodium | 0.0001-10% | 0.0001-10% | 0.0001-10% |
| magnesium | 0.0001-10% | 0.0001-10% | 0.0001-10% |
| chromium | 0.0001-10% | 0.0001-10% | 0.0001-10% |
| iodine | 0.0001-10% | 0.0001-10% | 0.0001-10% |
| iron | 0.0001-10% | 0.0001-10% | 0.0001-10% |
| manganese | 0.0001-10% | 0.0001-10% | 0.0001-10% |
| calcium | 0.0001-10% | 0.0001-10% | 0.0001-10% |
| copper | 0.0001-10% | 0.0001-10% | 0.0001-10% |
| fluoride | 0.0001-10% | 0.0001-10% | 0.0001-10% |
| potassium | 0.0001-10% | 0.0001-10% | 0.0001-10% |
| phosphorous | 0.0001-10% | 0.0001-10% | 0.0001-10% |
| molybdenum | 0.0001-10% | 0.0001-10% | 0.0001-10% |
| selenium | 0.0001-10% | 0.0001-10% | 0.0001-10% |
| zinc | 0.0001-10% | 0.0001-10% | 0.0001-10% |
| L-carnitine | 0.0001-10% | 0.0001-10% | 0.0001-10% |
| choline | 0.0001-10% | 0.0001-10% | 0.0001-10% |
| coenzyme Q10 | 0.0001-10% | 0.0001-10% | 0.0001-10% |
| alpha-lipoic acid | 0.0001-10% | 0.0001-10% | 0.0001-10% |
| omega-3-fatty acids | 0.0001-10% | 0.0001-10% | 0.0001-10% |
| pepsin | 0.0001-10% | 0.0001-10% | 0.0001-10% |
| phytase | 0.0001-10% | 0.0001-10% | 0.0001-10% |
| trypsin | 0.0001-10% | 0.0001-10% | 0.0001-10% |
| lipases | 0.0001-10% | 0.0001-10% | 0.0001-10% |
| proteases | 0.0001-10% | 0.0001-10% | 0.0001-10% |
| cellulases | 0.0001-10% | 0.0001-10% | 0.0001-10% |
| ascorbic acid | 0.0001-10% | 0.0001-10% | 0.0001-10% |
| citric acid | 0.0001-10% | 0.0001-10% | 0.0001-10% |
| rosemary oil | 0.0001-10% | 0.0001-10% | 0.0001-10% |
| vitamin A | 0.0001-10% | 0.0001-10% | 0.0001-10% |
| vitamin E phosphate | 0.0001-10% | 0.0001-10% | 0.0001-10% |
| tocopherols | 0.0001-10% | 0.0001-10% | 0.0001-10% |
| di-alpha-tocopheryl phosphate | 0.0001-10% | 0.0001-10% | 0.0001-10% |
| tocotrienols | 0.0001-10% | 0.0001-10% | 0.0001-10% |
| alpha lipoic acid | 0.0001-10% | 0.0001-10% | 0.0001-10% |
| dihydrolipoic acid | 0.0001-10% | 0.0001-10% | 0.0001-10% |
| xanthophylls | 0.0001-10% | 0.0001-10% | 0.0001-10% |
| beta cryptoxanthin | 0.0001-10% | 0.0001-10% | 0.0001-10% |
| lycopene | 0.0001-10% | 0.0001-10% | 0.0001-10% |
| lutein | 0.0001-10% | 0.0001-10% | 0.0001-10% |
| zeaxanthin | 0.0001-10% | 0.0001-10% | 0.0001-10% |
| beta-carotene | 0.0001-10% | 0.0001-10% | 0.0001-10% |
| carotenes | 0.0001-10% | 0.0001-10% | 0.0001-10% |
| mixed carotenoids | 0.0001-10% | 0.0001-10% | 0.0001-10% |
| polyphenols | 0.0001-10% | 0.0001-10% | 0.0001-10% |
| flavonoids | 0.0001-10% | 0.0001-10% | 0.0001-10% |
| cartotenoids | 0.0001-10% | 0.0001-10% | 0.0001-10% |
| chlorophyll | 0.0001-10% | 0.0001-10% | 0.0001-10% |
| chlorophyllin | 0.0001-10% | 0.0001-10% | 0.0001-10% |
| fiber | 0.0001-10% | 0.0001-10% | 0.0001-10% |
| anthocyanins | 0.0001-10% | 0.0001-10% | 0.0001-10% |
| cyaniding | 0.0001-10% | 0.0001-10% | 0.0001-10% |
| delphinidin | 0.0001-10% | 0.0001-10% | 0.0001-10% |
| malvidin | 0.0001-10% | 0.0001-10% | 0.0001-10% |
| pelargonidin | 0.0001-10% | 0.0001-10% | 0.0001-10% |
| peonidin | 0.0001-10% | 0.0001-10% | 0.0001-10% |
| petunidin | 0.0001-10% | 0.0001-10% | 0.0001-10% |
| flavanols | 0.0001-10% | 0.0001-10% | 0.0001-10% |
| flavonols | 0.0001-10% | 0.0001-10% | 0.0001-10% |
| catechin | 0.0001-10% | 0.0001-10% | 0.0001-10% |
| epicatechin | 0.0001-10% | 0.0001-10% | 0.0001-10% |
| epigallocatechin | 0.0001-10% | 0.0001-10% | 0.0001-10% |
| epigallocatechingallate | 0.0001-10% | 0.0001-10% | 0.0001-10% |
| theaflavins | 0.0001-10% | 0.0001-10% | 0.0001-10% |
| thearubigins | 0.0001-10% | 0.0001-10% | 0.0001-10% |
| proanthocyanins | 0.0001-10% | 0.0001-10% | 0.0001-10% |
| quercetin | 0.0001-10% | 0.0001-10% | 0.0001-10% |
| kaempferol | 0.0001-10% | 0.0001-10% | 0.0001-10% |
| myricetin | 0.0001-10% | 0.0001-10% | 0.0001-10% |
| isorhamnetin | 0.0001-10% | 0.0001-10% | 0.0001-10% |
| flavononeshesperetin | 0.0001-10% | 0.0001-10% | 0.0001-10% |
| naringenin | 0.0001-10% | 0.0001-10% | 0.0001-10% |
| eriodictyol | 0.0001-10% | 0.0001-10% | 0.0001-10% |
| tangeretin | 0.0001-10% | 0.0001-10% | 0.0001-10% |
| flavones | 0.0001-10% | 0.0001-10% | 0.0001-10% |
| apigenin | 0.0001-10% | 0.0001-10% | 0.0001-10% |
| luteolin | 0.0001-10% | 0.0001-10% | 0.0001-10% |
| lignans | 0.0001-10% | 0.0001-10% | 0.0001-10% |
| phytoestrogens | 0.0001-10% | 0.0001-10% | 0.0001-10% |
| resveratrol | 0.0001-10% | 0.0001-10% | 0.0001-10% |
| isoflavones | 0.0001-10% | 0.0001-10% | 0.0001-10% |
| daidzein | 0.0001-10% | 0.0001-10% | 0.0001-10% |
| genistein | 0.0001-10% | 0.0001-10% | 0.0001-10% |
| soy isoflavones | 0.0001-10% | 0.0001-10% | 0.0001-10% |
| L. Throat care actives | | | |
| (1) analgesics, anesthetics, | | | |
| antipyretic and anti- | | | |
| inflammatory agents | | | |
| menthol | 10-500 ppm | 10-500 ppm | 500-20,000 ppm |
| phenol | 0.1-10% | 0.1-50% | 0.1-20% |
| hexylresorcinol | 0.1-10% | 0.1-50% | 0.1-20% |
| benzocaine | 0.1-10% | 0.1-50% | 0.1-20% |
| dyclonine hydrochloride | 0.1-10% | 0.1-50% | 0.1-20% |
| benzyl alcohol | 0.1-10% | 0.1-50% | 0.1-20% |
| salicyl alcohol | 0.1-10% | 0.1-50% | 0.1-20% |
| acetaminophen | 0.1-10% | 0.1-50% | 0.1-20% |
| aspirin | 0.1-10% | 0.1-50% | 0.1-20% |
| diclofenac | 0.1-10% | 0.1-50% | 0.1-20% |
| diflunisal | 0.1-10% | 0.1-50% | 0.1-20% |
| etodolac | 0.1-10% | 0.1-50% | 0.1-20% |
| fenoprofen | 0.1-10% | 0.1-50% | 0.1-20% |
| flurbiprofen | 0.1-10% | 0.1-50% | 0.1-20% |
| ibuprofen | 0.1-10% | 0.1-50% | 0.1-20% |
| ketoprofen | 0.1-10% | 0.1-50% | 0.1-20% |
| ketorolac | 0.1-10% | 0.1-50% | 0.1-20% |
| nabumetone | 0.1-10% | 0.1-50% | 0.1-20% |
| naproxen | 0.1-10% | 0.1-50% | 0.1-20% |
| piroxicam | 0.1-10% | 0.1-50% | 0.1-20% |
| caffeine | 0.0001-10% | 0.0001-10% | 0.0001-10% |
| lidocaine | 0.1-10% | 0.1-50% | 0.1-20% |
| benzocaine | 0.1-10% | 0.1-50% | 0.1-20% |
| phenol | 0.1-10% | 0.1-50% | 0.1-20% |
| dyclonine | 0.1-10% | 0.1-50% | 0.1-20% |
| benzonotate | 0.1-10% | 0.1-50% | 0.1-20% |
| (2) demulcents | | | |
| slippery elm bark | 0.1-10% | 0.1-10% | 0.1-10% |
| pectin | 0.1-10% | 0.1-10% | 0.1-10% |
| gelatin | 0.1-10% | 0.1-10% | 0.1-10% |
| (3) antiseptics | | | |
| cetylpyridinium chloride | 0.01-1% | 0.01-1% | 0.01-1% |
| domiphen bromide | 0.01-1% | 0.01-1% | 0.01-1% |
| dequalinium chloride | 0.01-1% | 0.01-1% | 0.01-1% |
| (4) antitussives | | | |
| chlophedianol hydrochloride | 0.0001-2% | 0.0001-2% | 0.0001-2% |
| codeine | 0.0001-2% | 0.0001-2% | 0.0001-2% |
| codeine phosphate | 0.0001-2% | 0.0001-2% | 0.0001-2% |
| codeine sulfate | 0.0001-2% | 0.0001-2% | 0.0001-2% |
| dextromethorphan | 0.0001-2% | 0.0001-2% | 0.0001-2% |
| dextromethorphan | 0.0001-2% | 0.0001-2% | 0.0001-2% |
| hydrobromide | | | |
| diphenhydramine citrate | 0.0001-2% | 0.0001-2% | 0.0001-2% |
| diphenhydramine hydrochloride | 0.0001-2% | 0.0001-2% | 0.0001-2% |
| dextrorphan | 0.0001-2% | 0.0001-2% | 0.0001-2% |
| diphenhydramine | 0.0001-2% | 0.0001-2% | 0.0001-2% |
| hydrocodone | 0.0001-2% | 0.0001-2% | 0.0001-2% |
| noscapine | 0.0001-2% | 0.0001-2% | 0.0001-2% |
| oxycodone | 0.0001-2% | 0.0001-2% | 0.0001-2% |
| pentoxyverine | 0.0001-2% | 0.0001-2% | 0.0001-2% |
| (5) throat soothing agents | | | |
| honey | 0.5-25% | 0.5-90% | 0.5-15% |
| propolis | 0.1-10% | 0.1-10% | 0.1-10% |
| aloe vera | 0.1-10% | 0.1-10% | 0.1-10% |
| glycerine | 0.1-10% | 0.1-10% | 0.1-10% |
| menthol | 10-500 ppm | 10-500 ppm | 500-20,000 ppm |
| (6) cough suppressants | | | |
| codeine | 0.0001-2% | 0.0001-2% | 0.0001-2% |
| antihistamines | 0.0001-2% | 0.0001-2% | 0.0001-2% |
| dextromethorphan | 0.0001-2% | 0.0001-2% | 0.0001-2% |
| isoproterenol | 0.0001-2% | 0.0001-2% | 0.0001-2% |
| (7) expectorants | | | |
| ammonium chloride | 0.0001-2% | 0.0001-2% | 0.0001-2% |
| guaifenesin | 0.0001-2% | 0.0001-2% | 0.0001-2% |
| ipecac fluid extract | 0.0001-2% | 0.0001-2% | 0.0001-2% |
| potassium iodide | 0.0001-2% | 0.0001-2% | 0.0001-2% |
| (8) mucolytics | | | |
| acetylcycsteine | 0.0001-2% | 0.0001-2% | 0.0001-2% |
| ambroxol | 0.0001-2% | 0.0001-2% | 0.0001-2% |
| bromhexine | 0.0001-2% | 0.0001-2% | 0.0001-2% |
| (9) antihistamines | | | |
| acrivastine | 0.05-10% | 0.05-10% | 0.05-10% |
| azatadine | 0.05-10% | 0.05-10% | 0.05-10% |
| brompheniramine | 0.05-10% | 0.05-10% | 0.05-10% |
| chlorpheniramine | 0.05-10% | 0.05-10% | 0.05-10% |
| clemastine | 0.05-10% | 0.05-10% | 0.05-10% |
| cyproheptadine | 0.05-10% | 0.05-10% | 0.05-10% |
| dexbrompheniramine | 0.05-10% | 0.05-10% | 0.05-10% |
| dimenhydrinate | 0.05-10% | 0.05-10% | 0.05-10% |
| diphenhydramine | 0.05-10% | 0.05-10% | 0.05-10% |
| doxylamine | 0.05-10% | 0.05-10% | 0.05-10% |
| hydroxyzine | 0.05-10% | 0.05-10% | 0.05-10% |
| meclizine | 0.05-10% | 0.05-10% | 0.05-10% |
| phenindamine | 0.05-10% | 0.05-10% | 0.05-10% |
| phenyltoloxamine | 0.05-10% | 0.05-10% | 0.05-10% |
| promethazine | 0.05-10% | 0.05-10% | 0.05-10% |
| pyrilamine | 0.05-10% | 0.05-10% | 0.05-10% |
| tripelennamine | 0.05-10% | 0.05-10% | 0.05-10% |
| triprolidine | 0.05-10% | 0.05-10% | 0.05-10% |
| astemizole | 0.05-10% | 0.05-10% | 0.05-10% |
| cetirizine | 0.05-10% | 0.05-10% | 0.05-10% |
| ebastine | 0.05-10% | 0.05-10% | 0.05-10% |
| fexofenadine | 0.05-10% | 0.05-10% | 0.05-10% |
| loratidine | 0.05-10% | 0.05-10% | 0.05-10% |
| terfenadine | 0.05-10% | 0.05-10% | 0.05-10% |
| (10) nasal decongestants | | | |
| phenylpropanolamine | 0.1-10% | 0.1-50% | 0.1-20% |
| pseudoephedrine | 0.1-10% | 0.1-50% | 0.1-20% |
| ephedrine | 0.1-10% | 0.1-50% | 0.1-20% |
| phenylephrine | 0.1-10% | 0.1-50% | 0.1-20% |
| oxymetazoline | 0.1-10% | 0.1-50% | 0.1-20% |
| menthol | 0.1-10% | 0.1-50% | 0.1-20% |
| camphor | 0.1-10% | 0.1-50% | 0.1-20% |
| borneol | 0.1-10% | 0.1-50% | 0.1-20% |
| ephedrine | 0.1-10% | 0.1-50% | 0.1-20% |
| eucalyptus oil | 0.001-10% | 0.001-10% | 0.001-10% |
| peppermint oil | 0.001-10% | 0.001-10% | 0.001-10% |
| methyl salicylate | 0.001-10% | 0.001-10% | 0.001-10% |
| bornyl acetate | 0.001-10% | 0.001-10% | 0.001-10% |
| lavender oil | 0.001-10% | 0.001-10% | 0.001-10% |
| wasabi extracts | 0.001-10% | 0.001-10% | 0.001-10% |
| horseradish extracts | 0.001-10% | 0.001-10% | 0.001-10% |
| M. Tooth whitening/Stain | | | |
| removing agents | | | |
| surfactants | 0.001-2% | 0.001-2% | 0.001-2% |
| chelators | 0.1-10% | 0.1-10% | 0.1-10% |
| abrasives | 0.1-5% | 0.1-5% | 0.1-5% |
| oxidizing agents | 0.1-5% | 0.1-5% | 0.1-5% |
| hydrolytic agents | 0.1-5% | 0.1-5% | 0.1-5% |
| N. Energy boosting agents | | | |
| caffeine | 0.0001-10% | 0.0001-10% | 0.0001-10% |
| vitamins | 0.0001-10% | 0.0001-10% | 0.0001-10% |
| minerals | 0.0001-10% | 0.0001-10% | 0.0001-10% |
| amino acids | 0.0001-10% | 0.0001-10% | 0.0001-10% |
| ginseng extract | 0.0001-10% | 0.0001-10% | 0.0001-10% |
| ginko extract | 0.0001-10% | 0.0001-10% | 0.0001-10% |
| guarana extract | 0.0001-10% | 0.0001-10% | 0.0001-10% |
| green tea extract | 0.0001-10% | 0.0001-10% | 0.0001-10% |
| taurine | 0.0001-10% | 0.0001-10% | 0.0001-10% |
| kola nut extract | 0.0001-10% | 0.0001-10% | 0.0001-10% |
| yerba mate leaf | 0.0001-10% | 0.0001-10% | 0.0001-10% |
| Niacin | 0.0001-10% | 0.0001-10% | 0.0001-10% |
| rhodiola root extract | 0.0001-10% | 0.0001-10% | 0.0001-10% |
| O. Concentration boosting | | | |
| agents | | | |
| caffeine | 0.0001-10% | 0.0001-10% | 0.0001-10% |
| ginko extract | 0.0001-10% | 0.0001-10% | 0.0001-10% |
| gotu cola (centella asiatica) | 0.0001-10% | 0.0001-10% | 0.0001-10% |
| German chamomile | 0.0001-10% | 0.0001-10% | 0.0001-10% |
| avina sativa | 0.0001-10% | 0.0001-10% | 0.0001-10% |
| phosphatidyl serine | 0.0001-10% | 0.0001-10% | 0.0001-10% |
| aspalathus linearis | 0.0001-10% | 0.0001-10% | 0.0001-10% |
| pregnenolone | 0.0001-10% | 0.0001-10% | 0.0001-10% |
| rhodiola root extract | 0.0001-10% | 0.0001-10% | 0.0001-10% |
| theanine | 0.0001-10% | 0.0001-10% | 0.0001-10% |
| vinpocetine | 0.0001-10% | 0.0001-10% | 0.0001-10% |
| P. Appetite suppressants | | | |
| caffeine | 0.0001-10% | 0.0001-10% | 0.0001-10% |
| guarana extract | 0.0001-10% | 0.0001-10% | 0.0001-10% |
| hoodia gordonii | 0.0001-10% | 0.0001-10% | 0.0001-10% |
| glucomannan | 0.0001-10% | 0.0001-10% | 0.0001-10% |
| calcium | 0.0001-10% | 0.0001-10% | 0.0001-10% |
| garcinia cambogia extract | 0.0001-10% | 0.0001-10% | 0.0001-10% |
| n-acetyl-tyrosine | 0.0001-10% | 0.0001-10% | 0.0001-10% |
| soy phospholipids | 0.0001-10% | 0.0001-10% | 0.0001-10% |
| V. Colors | | | |
| Annatto extract | 0.5-10% | 0.5-20% | 0.5-10% |
| Beta-carotene | 0.5-10% | 0.5-20% | 0.5-10% |
| Canthaxanthin | 0.5-10% | 0.5-20% | 0.5-10% |
| Grape color extract | 0.5-10% | 0.5-20% | 0.5-10% |
| Turmeric oleoresin | 0.5-10% | 0.5-20% | 0.5-10% |
| B-Apo-8′-carotenal | 0.5-10% | 0.5-20% | 0.5-10% |
| Beet powder | 0.5-10% | 0.5-20% | 0.5-10% |
| Caramel color | 0.5-10% | 0.5-20% | 0.5-10% |
| Carmine | 0.5-10% | 0.5-20% | 0.5-10% |
| Cochineal extract | 0.5-10% | 0.5-20% | 0.5-10% |
| Grape skin extract | 0.5-10% | 0.5-20% | 0.5-10% |
| Saffron | 0.5-10% | 0.5-20% | 0.5-10% |
| Tumeric | 0.5-10% | 0.5-20% | 0.5-10% |
| Titanium dioxide | 0.05-2% | 0.05-2% | 0.05-2% |
| F.D. & C. Blue No. 1 | 0.05-2% | 0.05-2% | 0.05-2% |
| F.D.& C. Blue No. 2 | 0.05-2% | 0.05-2% | 0.05-2% |
| F.D.& C. Green No. 1 | 0.05-2% | 0.05-2% | 0.05-2% |
| F.D. & C. Red No. 40 | 0.05-2% | 0.05-2% | 0.05-2% |
| F.D. & C. Red No. 3 | 0.05-2% | 0.05-2% | 0.05-2% |
| F.D. & C. Yellow No. 6 | 0.05-2% | 0.05-2% | 0.05-2% |
| F.D. & C. Yellow No. 5 | 0.05-2% | 0.05-2% | 0.05-2% |

### Ingredient Release Management

In different embodiments, different techniques, ingredients, and/or delivery systems, may be used to manage release of one or more ingredients in an edible composition. In some embodiments, more than one of the techniques, ingredients, and/or delivery systems may be used.

In some embodiments, the delay in availability or other release of an ingredient in an edible composition caused by encapsulation of the ingredient may be based, in whole or in part, by one or more of the following: the type of encapsulating material, the molecular weight of the encapsulating material, the tensile strength of the delivery system containing the ingredient, the hydrophobicity of the encapsulating material, the particle size of the ingredient, the particle size of the delivery system, the presence of other materials in the edible composition (e.g., tensile strength modifying agents, emulsifiers), the ratio of the amounts of one or more ingredients in the delivery system to the amount of the encapsulating material in the delivery system, the order and/or amount of addition of one or more ingredients during mixing of the delivery system or edible composition, the number of layers of encapsulating material, the desired texture, flavor, shelf life, or other characteristic of edible composition, the ratio of the encapsulating material to the ingredient being encapsulated, etc. Thus, by changing or managing one or more of these characteristics of a delivery system or the edible composition, or the manufacturing method used to create the delivery system or the edible composition, release of one or more ingredients in an edible composition during consumption of the edible composition can be managed more effectively and/or a more desirable release profile for one or more ingredients in the delivery system may be obtained. This may lead to a more positive sensory or consumer experience during consumption of the product, more effective release of such one or more ingredients during consumption of the product, less need for the ingredient (e.g., more effective release of the ingredient may allow the amount of the ingredient in the edible composition to be reduced), increased delivery of a therapeutic or other functional benefit to the consumer, etc. Additionally, in some embodiments, managing the release rate or profile can be tailored to specific consumer segments.

In some embodiments, a method for managing release profile or one or more ingredients in a delivery system or in an edible composition containing the delivery system, may include measuring, estimating, or otherwise determining a partial or complete release profile for the one or more ingredients during consumption of delivery system or edible composition. Such a release profile may show one or more points of interest (e.g., flavor intensity, active availability, taste) over a period of time and/or at distinct points in time during consumption of a delivery system or an edible composition that includes the delivery system. Such a release profile may be obtained from a descriptive panel analysis, deduced or otherwise determined from an analytical chemistry analysis, and/or from other techniques known in the art. One example of a descriptive analysis technique is the Quantitative Descriptive Analysis (QDA™) method developed by Tragon Corp. (as described in SENSORY EVALUATION TECHNIQUES, 3^{RD} ED., MORTON MEILGAARD, GAIL CIVILLE, B. THOMAS CARR, EDS., CRC Press (1999), pp. 167-68). Another descriptive analysis technique is the Spectrum™ Descriptive Analysis Method developed by Civille (see SENSORY EVALUATION TECHNIQUES, 3^{RD}ED., pp. 168, 173-76.

In some embodiments, if it is desired to delay or sustain the release of at least a portion of one or more ingredients encapsulated in a delivery system as part of an edible composition, one or more of the following actions may be taken:

1. the tensile strength of the delivery system may be increased (e.g., by using a different encapsulating material that provides a higher tensile strength to the delivery system);

2. an encapsulating material having a higher molecular weight than the encapsulating material in the delivery system can be substituted for some or all of the encapsulated material in the delivery system;

3. an encapsulating material having a higher hydrophobicity than the encapsulating material in the delivery system can be substituted for some or all of the encapsulated material in the delivery system;

4. the ratio of components in the encapsulating material may be modified to increase the hydrophobicity of the encapsulating material;

5. the ratio of the amount encapsulating material in the delivery system to the amount of the one or more ingredients in the delivery system may be increased;

6. a different delivery system that includes the same one or more ingredients as the original delivery system in the edible composition and has a higher hydrophobicity and/or tensile strength than the original delivery system may be substituted for some or all of the original delivery system;

7. a different delivery system that includes the same one or more ingredients as the original delivery system in the edible composition and has a higher hydrophobicity and/or tensile strength than the original delivery system may be added to the edible composition;

8. the particle size of the delivery system in the edible composition may be increased (e.g., from 250 microns to 420 or 710 microns);

9. the amount tensile strength modifying agents in the delivery system or in the edible composition that reduce the tensile strength of the delivery system may be decreased;

10. the amount of an ingredient in the edible composition, but not the delivery system, may be decreased if the ingredient reacts or mixes with the delivery system or one of its components in an adverse manner or otherwise causes one of the components to release too early or too early;

11. another ingredient may be added to the edible composition that may cause additional release or availability of the one or more ingredients (this may be particularly beneficial when free amounts of the one or more ingredients are present in the edible composition, but do not release from the edible composition);

12. another ingredient may be added to the edible composition that may reduce or otherwise impact capture of the one or more ingredients in some other component (e.g., a chewing gum base) of the edible composition (e.g., a chewing gum), thereby increasing the amount of the one or more ingredients delivered or available to the consumer (this may be particularly beneficial when free amounts of the one or more ingredients are present in the edible composition, but do not release from the edible composition (e.g., they get trapped in the gum base of a chewing gum composition));

13. the edible composition can be manipulated to increase the mechanical pressure needed to chew the composition;

14. the delivery system can be more intimately mixed with the remaining ingredients in the edible composition;

15. the delivery system can be situated in the edible composition such that more time and/or effort are required to reach the delivery system during consumption (e.g., the delivery system can be located in an inner layer of a multilayer edible composition);

16. the delivery system may be encapsulated again in the same or a different encapsulating material;

17. a fixative can be added to the delivery system or to an edible composition that contains the delivery system, the fixative acting to change the vapor pressure or other characteristic of the ingredient so as to delay its release or otherwise extend its availability during consumption;

18. the delivery system can be partially or completed coated or treated with another material; and/or;

19. the one or more ingredients in the delivery system may be coated or otherwise pre-treated prior to encapsulation to increase the tensile strength and/or hydrophobicity of the delivery system, decrease the miscibility of the one or more ingredients with the encapsulating material, or otherwise stabilize the one or more ingredients prior to, during, and/or after the encapsulation process.

If it is desired to hasten the release of at least a portion of the one or more ingredients in the delivery system that is itself an ingredient in the edible composition, in some embodiments, one or more of the following actions may be taken:

1. the tensile strength of the delivery system may be decreased (e.g., by using a different encapsulating material that provides a lower tensile strength to the delivery system, by adding tensile strength modifying agents to the delivery system);

2. an encapsulating material having a lower molecular weight than the encapsulating material in the delivery system can be substituted for some or all of the encapsulated material in the delivery system;

3. an encapsulating material having a lower hydrophobicity than the encapsulating material in the delivery system can be substituted for some or all of the encapsulated material in the delivery system;

4. the ratio of components in the encapsulating material may be modified to decrease the hydrophobicity of the encapsulating material;

5. the ratio of the amount encapsulating material in the delivery system to the amount of the one or more ingredients in the delivery system may be decreased;

6. a different delivery system that includes the same one or more ingredients as the original delivery system in the edible composition and has a lower hydrophobicity and/or tensile strength than the original delivery system may be substituted for some or all of the original delivery system;

7. a different delivery system that includes the same one or more ingredients as the original delivery system in the edible composition and has a lower hydrophobicity and/or tensile strength than the original delivery system may be added to the edible composition;

8. the particle size of the ingredients in the delivery system may be decreased;

9. the particle size of the delivery system in the edible composition may be decreased;

10. the amount tensile strength modifying agents in the delivery system or in the edible composition that reduce the tensile strength of the delivery system may be increased;

11. the amount of an ingredient in the edible composition, but not the delivery system, may be increased if the ingredient reacts or mixes with the delivery system or one of its components in a way that causes one or more components to release faster or earlier;

12. another ingredient may be partially or completely removed from the edible composition if such removal will cause additional release or availability of the one or more ingredients;

13. the edible composition can be manipulated to decrease the mechanical pressure needed to chew the composition;

14. the delivery system can be less intimately mixed with the edible composition;

15. the delivery system can be situated in the edible composition such that less time and/or effort are required to reach the delivery system during consumption (e.g., the delivery system can be located in an outer layer of a multilayer edible composition);

16. another ingredient may be added to the edible composition that may increase or otherwise impact capture of the one or more ingredients in some other component (e.g., a chewing gum base) of the edible composition (e.g., a chewing gum), thereby decreasing the amount of the one or more ingredients delivered or available to the consumer; and/or

17. the one or more ingredients in the delivery system may be coated or otherwise pre-treated prior to encapsulation to decrease the tensile strength and/or hydrophobicity of the delivery system, increase the miscibility of the one or more ingredients with the encapsulating material, or otherwise destabilize the one or more ingredients prior to, during, and/or after the encapsulation process.

In some embodiments, in addition to or as an alternative to implementing one or more of the above changes, if it is desired to modify the release profile of at least a portion of one or more ingredients encapsulated in a delivery system as part of an edible composition, one or more of the following actions may be taken:

1. the amount of delivery system in the edible composition may be increased (which may serve to increase the intensity and/or duration of availability of the one or more ingredients during consumption of the edible composition);

2. the amount of delivery system in the edible composition may be decreased (which may serve to decrease the intensity and/or duration of availability of the one or more ingredients during consumption of the edible composition);

3. the process for mixing or otherwise making the delivery system can be modified;

4. the process for mixing or otherwise making the edible composition can be modified;

5. the average or maximum particle size of the ingredients in the delivery system can be increased;

6. the average or maximum particle size of the ingredients in the delivery system can be decreased;

7. the average particle size of the delivery system may be increased and the distribution of the average particle size of the delivery system can be sharpened or narrowed;

8. the average particle size of the delivery system may be increased and the distribution of the average particle size of the delivery system can be widened or made more smooth;

9. the average particle size of the delivery system may be decreased and the distribution of the average particle size of the delivery system can be sharpened or narrowed; and/or

10. the average particle size of the delivery system may be decreased and the distribution of the average particle size of the delivery system can be widened or made more smooth.

By using one or more of these techniques, the release of the one or more ingredients may be hastened or delayed as desired and/or the release profile of the one or more ingredients may be directed or otherwise managed towards a desired release profile, or at least a more desirable release profile. By trying various combinations of these techniques, as §0 desired, or at least more desirable, release profile can be obtained for the one or more ingredients in the edible composition. In some embodiments, obtaining such a desired release profile may include decreasing or increasing unencapsulated (i.e., free) amounts of the one or more ingredients in the edible composition and/or decreasing or increasing amounts of one or more additional delivery systems to the edible composition, wherein each of the delivery systems includes the one or more ingredients and is designed to release a predominant amount of the one or more ingredients at a desired time or during a desired time period following the start of consumption or other use of the edible composition. In some embodiments, the amount or location of a delivery system added to a mixing process for the delivery system or the edible composition, and/or the mixing time, also might be changed or experimented with to obtain a more desirable release profile for the one or more ingredients.

In some embodiments changes to amounts of two or more ingredients may be made in accordance with preferred or required ratios or equations. For example, oral compositions may need to balance acceptable germ kill properties and desirable taste characteristics. Adding too much of one or more germ killing ingredients in the oral composition may create a bad taste for the oral composition that will be unacceptable to the consumer. However, if not enough of the germ killing ingredient(s) are present in the oral composition, the oral composition may not function adequately as a germ killer or antimicrobial product. Thus, a balance may be created between the amount of the germ killing ingredient(s) in the oral composition and the flavor ingredients in the oral composition. Further examples of this can be found in U.S. patent application Ser. No. 11/010,082, the entire contents of which are incorporated herein by reference for all purposes.

In some embodiments, mixing limitations, ingredient limitations, technical requirements or limitations, ingredient availability, preferences or requirements regarding taste, texture, shelf life, mixing or other processing limitations or requirements, thermal stability and/or miscibility characteristics of one or more ingredients and or encapsulating materials, consumption duration, or other characteristic of the edible composition, consumer preference or acceptance criteria, implementation cost, government regulations, health concerns, etc., may limit the applicability of one or more of the techniques described herein. For example, in some embodiments, merely adding more of an ingredient (e.g., menthol, germ killing agents) may produce a bitter or bad taste that may be unacceptable to a consumer or not allowed under government regulations.

In some embodiments, a method for modifying a release profile of an ingredient in a delivery system, the delivery system being included in an edible composition, may include determining a first release profile for the ingredient; determining a desired change in release profile for the ingredient based on the first release profile; and modifying tensile strength of the delivery system based on the desired change in release profile for the ingredient. In some embodiments, the delivery system may include an encapsulating material with the ingredient being encapsulated with the encapsulating material. In some embodiments, the method may include one or more of the following: modifying hydrophobicity of the encapsulating material based on the desired change in release profile; modifying components of the encapsulating material to obtain a desired hydrophobicity of the encapsulating material; modifying a ratio of the ingredient to the encapsulating material based on the desired change in release profile; modifying an amount of the delivery system in the edible composition based on the desired change in release profile; modifying an unencapsulated amount of the ingredient in the edible composition based on the desired change in release profile; modifying average particle size of the delivery system in the edible composition based on the desired change in release profile; modifying maximum particle size of the delivery system in the edible composition based on the desired change in release profile; modifying average particle size of the ingredient based on the desired change in release profile; modifying maximum particle size of the ingredient based on the desired change in release profile.

In some embodiments, a method encapsulating an ingredient with an encapsulating material (or otherwise selecting the encapsulating material for the ingredient) may include determining a desired release profile for an ingredient in an edible composition; selecting an encapsulating material such that hydrophobicity of the encapsulating material and a tensile strength of a delivery system that will provide the desired release profile for the ingredient in the edible composition, wherein the delivery system includes the ingredient encapsulated with the encapsulating material; and encapsulating the ingredient with the encapsulating material.

In some embodiments, a method for modifying a release profile of an ingredient in a delivery system, the delivery system being included in an edible composition, may include determining a first release profile for the ingredient in the edible composition; determining a desired change in release profile for the ingredient based on the first release profile; and modifying at least one characteristic of the delivery system based on the desired change in release profile for the ingredient. In some embodiments, the characteristic of the delivery system may include one or more of the following: hydrophobicity of an encapsulating material used to encapsulate the ingredient; molecular weight of an encapsulating material used to encapsulate the ingredient; amount or other availability of a tensile strength modifying agent in the delivery system; amount of other availability of an emulsifier in the delivery system; ratio of an amount of the ingredient to an amount of an encapsulating material used to encapsulate the ingredient, average particle size of the delivery system; minimum or maximum particle size of the delivery system; average particle size of the ingredient; or minimum or maximum particle size of the ingredient.

In some embodiments, a method for modifying a release profile of an ingredient in a delivery system, the delivery system being included in an edible composition, may include determining an actual release profile for the ingredient in the edible composition; determining a desired change in release profile for the ingredient based on the actual release profile; and modifying at least one characteristic of the delivery system based on the desired change in release profile for the ingredient. In some embodiments, the delivery system may include the ingredient being encapsulated with an encapsulating material and modifying at least one characteristic of the delivery system may include one or more of the following: modifying tensile strength of the delivery system; modifying distribution of particle size of the delivery system; adding a fixative to the delivery system; modifying the encapsulating material to alter its hydrophobicity; modifying hydrophobicity of the encapsulating material; modifying a coating applied to the delivery system; modifying a coating applied to the ingredient before being encapsulated with the encapsulating material; modifying availability of a tensile strength modifying agent in the delivery system; modifying availability of an emulsifier in the delivery system; modifying availability of another ingredient in the delivery system; modifying ratio of the ingredient to the encapsulating material in the delivery system; modifying average particle size of the ingredient; modifying maximum particle size of the ingredient; modifying distribution of particle size of the delivery system; adding another layer of encapsulation to the delivery system; adding a hydrophilic coating to the delivery system; modifying minimum particle size of the delivery system; modifying average particle size of the delivery system; and modifying maximum particle size of the delivery system.

In some embodiments, a method for method for modifying a release profile of an ingredient in a delivery system, the delivery system being included in an edible composition, may include determining an actual release profile for the ingredient in the edible composition; determining a desired change in release profile for the ingredient based on the actual release profile; and modifying at least one characteristic of the edible composition based on the desired change in release profile for the ingredient.

In some embodiments, the delivery system may include the ingredient being encapsulated with an encapsulating material and modifying at least one characteristic of the edible composition may include one or more of the following: modifying tensile strength of the delivery system; modifying distribution of particle size of the delivery system; adding a fixative to the delivery system; modifying the encapsulating material to alter its hydrophobicity; modifying hydrophobicity of the encapsulating material; modifying availability of an emulsifier in the edible composition; modifying a coating applied to the delivery system; modifying a coating applied to the ingredient before being encapsulated with the encapsulating material; modifying availability of an unencapsulated amount of the ingredient in the edible composition; modifying availability of another ingredient in the edible composition; modifying availability of a tensile strength modifying agent in the delivery system; modifying availability of an emulsifier in the delivery system; modifying availability of another ingredient in the delivery system; modifying ratio of the ingredient to the encapsulating material in the delivery system; modifying average particle size of the ingredient; modifying maximum particle size of the ingredient; modifying distribution of particle size of the delivery system; adding another layer of encapsulation to the delivery system; adding a hydrophilic coating to the delivery system; modifying minimum particle size of the delivery system; modifying average particle size of the delivery system; and modifying maximum particle size of the delivery system.

In some embodiments, a method for modifying a release profile of an ingredient encapsulated with an encapsulating material in a delivery system, the delivery system being included in an edible composition, may include determining a first release profile for the ingredient; determining a desired change in release profile for the ingredient based on the first release profile; and modifying hydrophobicity the encapsulating material based on the desired change in release profile for the ingredient.

In some embodiments, a method for modifying a release profile of an ingredient encapsulated with an encapsulating material in a delivery system, the delivery system being included in an edible composition, may include determining a first release profile for the ingredient; determining a desired change in release profile for the ingredient based on the first release profile; and modifying ratio of the ingredient to the encapsulating material in the delivery system based on the desired change in release profile for the ingredient.

In some embodiments, a method for modifying a release profile of an ingredient encapsulated with an encapsulating material in a delivery system, the delivery system being included in an edible composition, may include determining a first release profile for the ingredient; determining a desired change in release profile for the ingredient based on the first release profile; and modifying average particle size of the delivery system in the edible composition based on the desired change in release profile.

In some embodiments, a method for managing a release profile of an ingredient in a delivery system, the delivery system being included in an edible composition, may include selecting a desired release profile of the ingredient; and selecting a tensile strength of the delivery system based on the desired release profile.

In some embodiments, a method for managing a release profile of an ingredient in a delivery system, the delivery system including the ingredient encapsulated with an encapsulating material and being included in an edible composition, may include selecting a desired release profile of the ingredient; and selecting a hydrophobicity of the encapsulating material based on the desired release profile.

In some embodiments, a method for managing a release profile of an ingredient in a delivery system, the delivery system including the ingredient encapsulated with an encapsulating material and being included in an edible composition, may include selecting a desired release profile of the ingredient; and selecting a ratio of the ingredient to the encapsulating material in the delivery system based on the desired release profile for the ingredient.

In some embodiments, a method for managing a release profile of an ingredient in a delivery system, the delivery system including the ingredient encapsulated with an encapsulating material and being included in an edible composition, may include selecting a desired release profile of the ingredient; and selecting a minimum, maximum, and/or average particle size of the delivery system in the edible composition based on the desired release profile.

In some embodiments, a method for managing a release profile of an ingredient in a delivery system, the delivery system including the ingredient encapsulated with an encapsulating material and being included in an edible composition, may include selecting a desired release profile of the ingredient; and selecting a distribution in the particle size of the delivery system in the edible composition based on the desired release profile.

In some embodiments, a method for managing a release profile of an ingredient in a delivery system, the delivery system including the ingredient encapsulated with an encapsulating material and being included in an edible composition, may include two or more of the following: selecting a desired release profile of the ingredient; selecting a ratio of the ingredient to the encapsulating material based on the desired release profile; selecting an tensile strength for the delivery system in the edible composition based on the desired release profile; selecting a hydrophobicity for the encapsulating material based on the desired release profile; and selecting an average particle size of the delivery system in the edible composition based on the desired release profile.

In some embodiments, a method for managing a release profile of an ingredient in a delivery system, the delivery system including the ingredient encapsulated with an encapsulating material and being included in an edible composition, may include selecting a desired release profile of the ingredient; and selecting a coating for the delivery system based on the desired release profile.

In some embodiments, a method for managing a release profile of an ingredient in a delivery system, the delivery system including the ingredient encapsulated with an encapsulating material and being included in an edible composition, may include selecting a desired release profile of the ingredient; and selecting a coating for the ingredient based on the desired release profile.

In some embodiments, a method for managing a release profile of an ingredient in a delivery system, the delivery system including the ingredient encapsulated with an encapsulating material and being included in an edible composition, may include selecting at least one of the following: tensile strength of the delivery system; distribution of particle size of the delivery system; a fixative for the delivery system; hydrophobicity of the encapsulating material; availability of a tensile strength modifying agent in the delivery system; availability of an emulsifier in the delivery system; ratio of the ingredient to the encapsulating material in the delivery system; average particle size of the ingredient; maximum particle size of the ingredient; a coating for the ingredient; a coating for the delivery system; another layer of encapsulation to be added to the delivery system; a hydrophilic coating to be added to the delivery system; minimum particle size of the delivery system; average particle size of the delivery system; and maximum particle size of the delivery system; and then making the delivery system. In some embodiments, the method also may include making an edible composition that includes the delivery system.

### Encapsulation

In some embodiments, one or more ingredients may be encapsulated with an encapsulating material. In general, partially or completely encapsulating an ingredient used in an edible composition with an encapsulating material may delay release of the ingredient during consumption of the edible composition, thereby delaying when the ingredient becomes available inside the consumer's mouth, throat, and/or stomach, available to react or mix with another ingredient, and/or available to provide some sensory experience and/or functional or therapeutic benefit. This can be particularly true when the ingredient is water soluble or at least partially water soluble.

In some embodiments, a material used to encapsulate an ingredient may include water insoluble polymers, co-polymers, or other materials capable of forming a strong matrix, solid coating, or film as a protective barrier with or for the ingredient. In some embodiments, the encapsulating material may completely surround, coat, cover, or enclose an ingredient. In other embodiments, the encapsulating material may only partially surround, coat, cover, or enclose an ingredient. Different encapsulating materials may provide different release rates or release profiles for the encapsulated ingredient. In some embodiments, encapsulating material used in a delivery system may include one or more of the following: polyvinyl acetate, polyethylene, crosslinked polyvinyl pyrrolidone, polymethylmethacrylate, polylactidacid, polyhydroxyalkanoates, ethylcellulose, polyvinyl acetatephthalate, polyethylene glycol esters, methacrylicacid-co-methylmethacrylate, ethylene-vinylacetate (EVA) copolymer, and the like, and combinations thereof.

In some embodiments, an ingredient may be pre-treated prior to encapsulation with an encapsulating material. For example, an ingredient may be coated with a “coating material” that is not miscible with the ingredient or is at least less miscible with the ingredient relative to the ingredient's miscibility with the encapsulating material.

In some embodiments, an encapsulation material may be used to individually encapsulate different ingredients in the same edible composition. For example, a delivery system may include aspartame encapsulated by polyvinyl acetate. Another delivery system may include ace-k encapsulated by polyvinyl acetate. Both delivery systems may be used as ingredients in the same chewing gum or in other edible compositions. For addition examples, see U.S. patent application Ser. No. 11/134,367 entitled “A Delivery System for Active Components as Part of an edible Composition” and filed May 23, 2005, the entire contents of which are incorporated herein by reference for all purposes.

In some embodiments, different encapsulation materials may be used to individually encapsulate different ingredients used in the same edible composition. For example, a delivery system may include aspartame encapsulated by polyvinyl acetate. Another delivery system may include ace-k encapsulated by EVA. Both delivery systems may be used as ingredients in the same chewing gum or other edible compositions. Examples of encapsulated ingredients using different encapsulating materials can be found in U.S. Patent Application Ser. No. 60/655,894 filed Feb. 25, 2005, and entitled “Process for Manufacturing a Delivery System for Active Components as Part of an Edible Composition,” the entire contents of which are incorporated herein by reference for all purposes.

### Methods of Encapsulation

There are many ways to encapsulate one or more ingredients with an encapsulating material. For example, in some embodiments, a sigma blade or Banbury™ type mixer may be used. In other embodiments, an extruder or other type of continuous mixer may be used. In some embodiments, spray coating, spray chilling, absorption, adsorption, inclusion complexing (e.g., creating a flavor/cyclodextrin complex, forming a glassy matrix, etc.), coacervation, fluidized bed coating, or other process may be used to encapsulate an ingredient with an encapsulating material.

Examples of encapsulation of ingredients can be found in U.S. Patent Application Ser. No. 60/655,894, filed Feb. 25, 2005, and entitled “Process for Manufacturing a Delivery System for Active Components as Part of an Edible Composition,” the entire contents of which are incorporated herein by reference for all purposes. Other examples of encapsulation of ingredients can be found in U.S. patent application Ser. No. 10/955,255 filed Sep. 30, 2004, and entitled “Encapsulated Compositions and Methods of Preparation,” the entire contents of which are incorporated herein by reference for all purposes. Further examples of encapsulation of ingredients can be found in U.S. patent application Ser. No. 10/955,149 filed Sep. 30, 2004, and entitled “Thermally Stable High Tensile Strength Encapsulation Compositions for Actives,” the entire contents of which are incorporated herein by reference for all purposes. Still further examples of encapsulation of ingredients can be found in U.S. patent application Ser. No. 11/052,672 filed Feb. 7, 2005, and entitled “Stable Tooth Whitening Gum with Reactive Components,” the entire contents of which are incorporated herein by reference for all purposes. Further encapsulation techniques and resulting delivery systems may be found in U.S. Pat. Nos. 6,770,308, 6,759,066, 6,692,778, 6,592,912, 6,586,023, 6,555,145, 6,479,071, 6,472,000, 6,444,241, 6,365,209, 6,174,514, 5,693,334, 4,711,784, 4,816,265, and 4,384,004, the contents of all of which are incorporated herein by reference for all purposes.

In some embodiments, a delivery system may be ground to a particular size for use as an ingredient in an edible composition. For example, in some embodiments, an ingredient may be ground to 710, 420, or 250 microns. In some embodiments, the delivery system may be ground to an average particle size such as, for example, 710, 420, or 250 microns. In some embodiments, the delivery system may be ground to a maximum particle size such as, for example, 710, 420, or 250 microns. The ultimate particle size will depend on the characteristics of the delivery system and/or the edible composition and as such, other sizes are possible in other embodiments. For example, delivery systems and/or edible compositions with smooth, creamy textures require smaller particles sizes (below 125 microns). Also, in some embodiments, particles below a certain size (e.g., 125 microns) may be removed. In some embodiments, the particle size distribution can have a narrow range resulting in a sharp distribution. In some embodiments, the particle size distribution can have a wide range resulting in a smooth distribution.

Tensile Strength

In some embodiments, selection of an encapsulating material for one or more ingredients may be based on tensile strength desired for the resulting delivery system. For example, in some embodiments, a delivery system produces delayed or otherwise controlled release of an ingredient through the use of a pre-selected or otherwise desired tensile strength.

In some embodiments, increasing the tensile strength of a delivery system may increase the delayed or extended release of an ingredient in the delivery system. The tensile strength for a delivery system may be matched with a desirable release rate selected according to the type of the ingredient(s) to be encapsulated for the delivery system, the encapsulating material used, any other additives incorporated in the delivery system and/or an edible composition using the delivery system as an ingredient, the desired rate of release of the ingredient, and the like. In some embodiments, the tensile strength of a delivery system which can be at least 6,500 psi, including 7500, 10,000, 20,000, 30,000, 40,000, 50,000, 60,000, 70,000, 80,000, 90,000, 100,000, 125,000, 135,000, 150,000, 165,000, 175,000, 180,000, 195,000, 200,000 and all ranges and subranges there between, for example, a tensile strength range of 6,500 to 200,000 psi.

In some embodiments, a delivery system for one or more ingredients can be provided based on the tensile strength of the delivery system having a specific tensile strength when compared to a standard. Thus, the design of the delivery system is not focused on one characteristic (e.g., molecular weight) of one of the materials (e.g., encapsulating material) used to produce the delivery system. In this manner, a delivery system can be formulated to express a desired release profile by adjusting and modifying the tensile strength through the specific selection of the ingredient(s), encapsulating material, additives, amount of the ingredient(s), amount of encapsulating material, relative amounts of ingredient(s) to encapsulating material, etc. If a desired tensile strength is chosen for a delivery system, any delivery system that has the desired tensile strength may be used without being limited to a particular encapsulating material and its molecular weight. The formulation process can be extended to encapsulating materials that exhibit similar physical and chemical properties as the encapsulating material forming part of the standard delivery system.

In some embodiments, a delivery system for delivering an ingredient may be formulated to ensure an effective sustained release of the ingredient based on the type and amount of the ingredient and the desired release rate for the ingredient. For example, it may be desirable to affect the controlled release of a high intensity sweetener from a chewing gum over a period of twenty-five to thirty minutes to ensure against a rapid burst of sweetness that may be offensive to some consumers. A shorter controlled release time may be desirable for other type of ingredients such as pharmaceuticals or therapeutic agents, which may be incorporated into the same edible composition by using separate delivery systems for each of these ingredients. Delivery systems may be formulated with a particular tensile strength associated with a range of release rates based on a standard. The standard may comprise a series of known delivery systems having tensile strengths over a range extending, for example, from low to high tensile strength values. Each of the delivery systems of the standard will be associated with a particular release rate or ranges of release rates. Thus, for example, a delivery system can be formulated with a relatively slow release rate by a fabricating a delivering system having a relatively high tensile strength. Conversely, lower tensile strength compositions tend to exhibit relatively faster release rates.

In some embodiments, an edible composition may include a plurality of delivery systems to deliver a plurality of separate ingredients, including ingredients that may be desirably released at distinctly different release rates. Each of the delivery systems may have a different tensile strength. For example, high intensity sweeteners may desirably be released over an extended period of time (e.g., twenty to thirty minutes) while some pharmaceuticals are desirably released over a significantly shorter period of time.

In some embodiments, a delivery system can be prepared such that the release of one or more ingredients in the delivery system agent is at specific rates relative to the time of delivery. For example, in one embodiment, a delivery system can be prepared such that at least one ingredient is released at a rate of 80% over the course of fifteen minutes, 90% over the course of twenty minutes, and/or a 95% over the course of thirty minutes. In another embodiment, the delivery system can be prepared such that one or more ingredients are released at a rate of 25% over the course of fifteen minutes, 50% over the course of twenty minutes and/or 75% over the course of thirty minutes.

In some embodiments, encapsulating material in a delivery system may be present in amounts of from about 0.2% to 10% by weight based on the total weight of the edible composition, including 0.3, 0.5, 0.7, 0.9, 1.0, 1.25, 1.4, 1.7, 1.9, 2.2, 2.45, 2.75, 3.0, 3.5, 4.0, 4.25, 4.8, 5.0, 5.5, 6.0, 6.5, 7.0, 7.25, 7.75, 8.0, 8.3, 8.7, 9.0, 9.25, 9.5, 9.8 and all values and ranges there between, for example, from 1% to 5% by weight. The amount of the encapsulating material can depend in part on the amount of the ingredient(s) component that is encapsulated. The amount of the encapsulating material with respect to the weight of the delivery system, is from about 30% to 99%, including 35, 40, 45, 50, 55, 60, 65, 70, 75, 80, 85, 95, 97 and all values and ranges there between, for example, from about 60% to 90% by weight.

In some embodiments, the tensile strength of a delivery system may be selected from relatively high tensile strengths when a relatively slow rate of release for an ingredient in the delivery system is desired and relatively lower tensile strengths when a faster rate of release for an ingredient in the delivery system is desired. Thus, when employing a tensile strength of 50,000 psi for a delivery system, the release rate of the ingredient, will generally be lower than the release rate of the ingredient in a delivery system having a tensile strength of 10,000 psi regardless of the type of encapsulating material (e.g., polyvinyl acetate) chosen.

In some embodiments, the encapsulating material for a delivery system is polyvinyl acetate. A representative example of a polyvinyl acetate product suitable for use as an encapsulating material in the present invention is Vinnapas® B100 sold by Wacker Polymer Systems of Adrian, Mich. A delivery system utilizing polyvinyl acetate may be prepared by melting a sufficient amount of polyvinyl acetate at a temperature of about 65° C. to 120° C. for a short period of time, e.g., five minutes. The melt temperature will depend on the type and tensile strength of the polyvinyl acetate encapsulating material where higher tensile strength materials will generally melt at higher temperatures. Once the encapsulating material is melted, a suitable amount of an ingredient (e.g., high intensity sweetener such as aspartame) is added and blended into the molten mass thoroughly for an additional short period of mixing. The resulting mixture is a semi-solid mass, which is then cooled (e.g., at 0° C.) to obtain a solid, and then ground to a U.S. Standard sieve size of from about 30 to 200 (600 to 75 microns). The tensile strength of the resulting delivery system can readily be tested according to ASTM-D638.

For additional information regarding how tensile strength of a delivery system may be used to create managed release of one or more ingredients, see U.S. patent application Ser. No. 11/083,968 entitled “A Delivery System for Active Components as Part of an Edible Composition Having Preselected Tensile Strength” and filed on Mar. 21, 2005, and U.S. patent application Ser. No. 10/719,298 entitled “A Delivery System for Active Components as Part of an Edible Composition” and filed Nov. 21, 2003, the complete contents of both of which are incorporated herein by reference for all purposes.

In some embodiments, a delivery system, and/or an edible composition having the delivery system as one of its ingredients, may include one or more additives that act as tensile strength modifying agents for the delivery system. For example, in some embodiments, the formulation of a delivery system with a desirable tensile strength can be made from a variety of encapsulating materials and at least one additive that acts as a tensile strength modifying agent. The additive may be added to the delivery system and/or to the edible composition containing the delivery system. The at least one additive may be used to formulate the delivery system by modifying the tensile strength of the delivery system, including tensile strength-lowering materials such as fats, emulsifiers, plasticizers (softeners), waxes, low molecular weight polymers, and the like, in addition to tensile strength increasing materials such as high molecular weight polymers. In addition, the tensile strength of the delivery system can also be fine tuned by combining different tensile strength modifiers to form the delivery system. For example, the tensile strength of high molecular weight polymers such as polyvinyl acetate may be reduced when tensile strength lowering agents such as fats and/or oils are added. Thus, by employing tensile strength modifiers, the overall tensile strength of the delivery system can be adjusted or altered in such a way that a pre-selected or otherwise desired tensile strength is obtained for the corresponding desired release rate of the ingredient from an edible composition based on a comparison with a standard.

Examples of tensile strength modifiers or modifying agents include, but are not limited to, fats (e.g., hydrogenated or non-hydrogenated vegetable oils, animal fats), waxes (e.g., microcrystalline wax, bees wax), plasticizers/emulsifiers (e.g., mineral oil, fatty acids, mono- and diglycerides, triacetin, glycerin, acetylated monoglycerides, glycerol rosin monostearate esters), low and high molecular weight polymers (e.g., polypropylene glycol, polyethylene glycol, polyisobutylene, polyethylene, polyvinylacetate) and the like, and combinations thereof. Plasticizers may also be referred to as softeners.

For additional information regarding use of tensile strength modifying agents for a delivery system to create managed release of one or more ingredients, see U.S. patent application Ser. No. 11/083,968 entitled “A Delivery System for Active Components as Part of an Edible Composition Having Preselected Tensile Strength” and filed on Mar. 21, 2005, and U.S. patent application Ser. No. 10/719,298 entitled “A Delivery System for Active Components as Part of an Edible Composition” and filed Nov. 21, 2003, the complete contents of both of which are incorporated herein by reference for all purposes.

### Hydrophobicity

In some embodiments, the release of one or more ingredients from a delivery system may depend on more than tensile strength. For example, the release of the ingredients may be directly related to the tensile strength of the delivery system and the hydrophobicity (i.e., water resistance) of the encapsulating polymer or other material.

As a more specific example, when a delivery system is used in a chewing gum, moisture may be absorbed in the encapsulated ingredient(s) during mastication and chewing of the chewing gum. This may result in softening of the encapsulating material and releasing of the ingredient(s) during the mastication and chewing of the chewing gum. The softening of the encapsulation material depends on the hydrophobicity of the polymer used as the encapsulation material. In general, the higher the hydrophobicity of the polymer, the longer mastication time is needed for softening the polymer.

As one example, higher hydrophobic polymers such as ethylene-vinylacetate (EVA) copolymer can be used to increase or otherwise manage ingredient (e.g., sweetener) release times from encapsulations. The degree of hydrophobicity can be controlled by adjusting the ratio of ethylene and vinylacetate in the copolymer. In general, the higher the ethylene to vinylacetate ratio, the longer time it will take during consumption to soften the encapsulation particles, and the slower or more delayed will be the release rate of the ingredient. The lower the ethylene to vinylacetate ratio, the shorter time it will take during consumption to soften the encapsulation particles, and the faster or earlier will be the release rate of the ingredient.

As illustrated by the discussion above, in some embodiments, release of an ingredient from a delivery system can be managed or otherwise controlled by formulating the delivery system based on the hydrophobicity of the encapsulating material, e.g., the polymer, for the ingredient. Using highly hydrophobic polymers, the release times of the ingredient can be increased or delayed. In a similar manner, using encapsulating material that is less hydrophobic, the ingredient can be released more rapidly or earlier.

The hydrophobicity of a polymer can be quantitated by the relative water-absorption measured according to ASTM D570-98. Thus, by selecting encapsulating material(s) for a delivery system with relatively lower water-absorption properties and adding that to a mixer, the release of the ingredient contained in the produced delivery system can be delayed compared to those encapsulating materials having higher water-absorption properties.

In some embodiments, polymers with water absorption of from about 50 to 100% (as measured according to ASTM D570-98) can be used. Moreover, to decrease the relative delivery rate, the encapsulating material can be selected such that the water absorption would be from about 15% to about 50% (as measured according to ASTM D570-98). Still further, in other embodiments, the water absorption properties of the encapsulating material can be selected to be from 0.0% to about 5% or up to about 15% (as measured according to ASTM D570-98). In other embodiments, mixtures of two or more delivery systems formulated with encapsulating material having different water-absorption properties can also be used in subsequent incorporation into an edible composition.

Polymers with suitable hydrophobicity which may be used for delivery systems include homo- and co-polymers of, for example, vinyl acetate, vinyl alcohol, ethylene, acrylic acid, methacrylate, methacrylic acid and others. Suitable hydrophobic copolymers include the following non-limiting examples, vinyl acetate/vinyl alcohol copolymer, ethylene/vinyl alcohol copolymer, ethylene/acrylic acid copolymer, ethylene/methacrylate copolymer, ethylene/methacrylic acid copolymer.

In some examples, the hydrophobic encapsulating material in a delivery system may be present in amounts of from about 0.2% to 10% by weight based on the total weight of an edible composition containing the delivery system, including 0.3, 0.5, 0.7, 0.9, 1.0, 1.25, 1.4, 1.7, 1.9, 2.2, 2.45, 2.75, 3.0, 3.5, 4.0, 4.25, 4.8, 5.0, 5.5, 6.0, 6.5, 7.0, 7.25, 7.75, 8.0, 8.3, 8.7, 9.0, 9.25, 9.5, 9.8 and all values and ranges there between, for example, from 1% to 5% by weight. The amount of the encapsulating material will, of course, depend in part on the amount of the ingredient that is encapsulated. The amount of the encapsulating material with respect to the weight of the delivery system, is from about 30% to 99%, including 35, 40, 45, 50, 55, 60, 65, 70, 75, 80, 85, 95, 97 and all values and ranges there between, for example, from about 60% to 90% by weight.

In formulating the delivery system based on the selection criteria of hydrophobicity of the encapsulating material, the encapsulated ingredient can be entirely encapsulated within the encapsulating material or incompletely encapsulated within the encapsulating material provided the resulting delivery system meets the criteria set forth hereinabove. The incomplete encapsulation can be accomplished by modifying and/or adjusting the manufacturing process to create partial coverage of the ingredient.

For example, if ethylene-vinyl acetate is the encapsulating material for an ingredient, the degree of hydrophobicity can be controlled by adjusting the ratio of ethylene and vinyl acetate in the copolymer. The higher the ethylene to vinylacetate ratio, the slower the release of the ingredient. Using vinylacetate/ethylene copolymer as an example, the ratio of the vinylacetate/ethylene in the copolymer can be from about 1 to about 60%, including ratios of 2.5, 5, 7.5, 9, 12, 18, 23, 25, 28, 30, 35, 42, 47, 52, 55, 58.5% and all values and ranges there between.

In some embodiments, a method of selecting a target delivery system containing an ingredient for an edible composition is based on the hydrophobicity of the encapsulating material for the ingredient in the delivery system. The method generally includes preparing a targeted delivery system containing an ingredient to be encapsulated, an encapsulating material and optional additives, with the encapsulating material having a pre-selected or otherwise desired hydrophobicity. The hydrophobicity of the encapsulating material employed in the targeted delivery system can be selected to provide a desirable release rate of the ingredient. This selection of the encapsulating material is based on the hydrophobicity of sample delivery systems having the same or similar ingredient and known release rates of the ingredient. In a more preferred another embodiment of the invention, the method comprises (a) obtaining a plurality of sample delivery systems comprising at least one ingredient, at least one encapsulating material, and optional additives, wherein each of the delivery systems is prepared with different encapsulating materials having different hydrophobicities; (b) testing the sample delivery systems to determine the respective release rates of the ingredient(s); and (c) formulating a target delivery system containing the same ingredient(s) with a hydrophobic encapsulating material corresponding to a desired release rate of the ingredient(s) based on the obtained sample delivery systems.

The method of selecting at least one delivery system suitable for incorporation into an edible composition preferably can begin by determining a desired release rate for an ingredient (i.e., a first active component). The determination of the desired release rate may be from known literature or technical references or by in vitro or in vivo testing. Once the desired release rate is determined, the desired hydrophobicity of the encapsulating material can be determined (i.e., a first hydrophobic encapsulating material) for a delivery system (i.e., first delivery system) that can release the first active component at the desired release. Once the delivery system is obtained which can deliver the first active component as required it is then selected for eventual inclusion in an edible composition.

The method described above may then be repeated for a second active component and for additional active components as described via the determination and selection of a suitable delivery system.

For additional information regarding the relationship of hydrophobicity of an encapsulating material to the release of an ingredient from a delivery system, see U.S. patent application Ser. No. 11/134,364 entitled “A Delivery System For Active Components as Part of an edible Composition” and filed on May 23, 2005, with the U.S. Patent and Trademark Office, the complete contents of which are incorporated herein by reference for all purposes.

### Ratio of Ingredient to Encapsulating Material for Ingredient in Delivery System

In general, the “loading” of an ingredient in a delivery system can impact the release profile of the ingredient when the ingredient is used in an edible composition. Loading refers to the amount of one or more ingredients contained in the delivery relative to the amount of encapsulating material. More specifically, the ratio of the amount of one or more ingredients in a delivery system to the amount of encapsulating material in the delivery system can impact the release rate of the one or more ingredients. For example, the lower the ratio or loading of the amount of one or more ingredients in a delivery system to the amount of encapsulating material in the delivery system, the longer or more delayed will be the release of the one or more ingredients from the delivery system. The higher the ratio or loading of the amount of one or more ingredients in a delivery system to the amount of encapsulating material in the delivery system, the faster or earlier will be the release of the one or more ingredients from the delivery system. This principle can be further employed to manage the release profiles of the one or more ingredients by using higher loading of ingredients designed to be released early in combination with lower loading of ingredients designed to be released later. In some embodiments, the one or more ingredients can be the same or different.

As a more specific example, three delivery systems including aspartame encapsulated with a polyvinylacetate and a fat were created using a conventional mixing process wherein the polyvinyl acetate first was melted in a mixer. The aspartame and fat then were added and the three ingredients were mixed to create a homogenous mixture. The delivery systems had the following aspartame to polyvinyl to fat ratios: (1) 5:90:5; (2) 15:80:5, (3) 30:65:5. The molten delivery systems were cooled and sized by passing ground powder through a 420 micron screen. Three chewing gums where created, each using a different delivery system. It was determined than the chewing gum using the first ratio of the ingredients had a lower or slower release of aspartame that the chewing gums using the second or third ratios of the ingredients. Similarly, the gum using the second ratio of the ingredients had a lower or slower release of aspartame than the chewing gum using the third ratio of the ingredients.

For additional information regarding the relationship of the ratio of the amount ingredient in a delivery system to the amount of encapsulating material in the delivery system to the release of an ingredient from a delivery system, see U.S. patent application Ser. No. 11/134,371 entitled “A Delivery System For Active Components as Part of an edible Composition” and filed on May 23, 2005, with the U.S. Patent and Trademark Office, the complete contents of which are incorporated herein by reference for all purposes.

### Change or Manage Characteristic of Ingredient

In some embodiments, the vapor pressure of the one or more ingredients can be manipulated to affect release of the one or more ingredients. For example, a volatile material can be combined with a fixative to decrease its vapor pressure and delay release from the delivery system. Examples of materials that can be used as fixatives include, but are not limited to sequiterpenes such as viridiflorol, poly limonene, sucrose acetate isobutyrate (SAIB), ester gum, ethyl cellulose or related polymers, hydrocolloids, vegetable oils, medium chain triglycerides, triethyl citrate, triglycerides such as triacetin and the like, glycerin, and propylene glycol.

In some embodiments, the phase of the one or more ingredients can be changed to affect release. For example, liquid ingredients can be processed into solid materials prior to encapsulation. In some embodiments, the one or more liquid ingredients can be processed by spray drying, spray chilling, fluidized bed drying, coacervation, absorption, adsorption, or inclusion processed to form complexes with cyclodextrins or glasses such as sucrose, maltodextrin, polyols, and the like.

### Variations in Particle Size of Ingredient or Delivery System

In some embodiments, release of an ingredient (e.g., a sweetener) in an edible composition can be modified or otherwise managed by varying particle size and distribution of the delivery system that includes the ingredient. For example, smaller particle size and sharper particle size distribution of a delivery system will result in faster or earlier release of the encapsulated ingredient as compared to delivery systems having bigger particle sizes and wider distributions. A sharp particle size distribution can be obtained by having a more narrow range of particle sizes. Increasing the particle size of the delivery system will delay the release of the encapsulated ingredient. Also, smoothing the particle size distribution can provide sustained release. A particle size distribution can be smoothed by expanding the range of particle sizes. This principle can also be applied to design delivery systems that manage release profile. For example, one or more ingredients in delivery systems with smaller, sharper particle sizes can be combined with one or more ingredients in delivery systems with larger, smoother particles to provide both faster and delayed release of the respective one or more ingredients.

As a more specific example, a delivery system can include the following ingredients by percentage: aspartame (30%), polyvinylacetate (65%), hydrogenated oil (3.75%), glycerolmonstearate (1.25%). The polyvinylacetate can be melted in a twin screw extruder. Hydrogenated oil and glycerolmonostearate are mixed under high shear in the extruder and dispersed completely in the polymer melt. The molten encapsulation blends are cooled and sized by passing ground powder through three different particle size screens. For example, the screens may be 250, 420 and 710 microns sizes. Particles passing through each screen can then be collected and used. When the particles passing through the screen are collected and used, the screen size becomes the maximum particle size for the material. Very small particles from all the three sized powders can be removed by passing the powders through a 125 micron screen and removing the particles that Dass through the 125 micron screen.

For gums prepared containing the above mentioned encapsulated aspartame particles, aspartame released in the order of smallest to largest maximum particle size (i.e., 250>420>710 micron particle sizes). The larger the size of the delivery system, the more delayed or the slower the release. By changing the particle size of the encapsulated aspartame (e.g., the size of the delivery system), the release of the aspartame in chewing gum can be managed to create or approximate a desired release profile.

For additional information regarding the relationship of particle size of a delivery system to the release of an ingredient from the delivery system, see U.S. patent application Ser. No. 11/134,480 entitled “A Delivery System for Active Components as Part of an Edible Composition Having Selected Particle Size” and filed on May 23, 2005, the complete contents of which are incorporated herein by reference for all purposes.

### Pre-treatment of an Ingredient Prior to Encapsulation

In some embodiments, some or all of the ingredient(s) encapsulated within an encapsulating material may be miscible with the encapsulating material. For example, polyvinylacetate is one type of encapsulating material that can be used in some embodiments. Some components, such as flavors comprising short or medium chain esters, may interact with the polyvinylacetate (PVA) and thereby reduce the effectiveness of the controlled and/or delayed release profile of the ingredient. In addition, or alternatively, to the issue of miscibility, one or more of the ingredients may be sensitive to heat and may become compromised, lose effectiveness, or otherwise be damaged when exposed to heat. For example, the ingredients may be subjected to heat during the encapsulation process.

Therefore, in some embodiments, by itself or combined with the other embodiments described herein, an ingredient is coated with a “coating material” that is not miscible or at least less miscible relative to its miscibility with the encapsulating material. The coating also may thermally stabilize the encapsulated ingredient(s) or at least make them less sensitive to the application of heat. The ingredient can be treated with the coating material prior to or concurrently with its encapsulation with the encapsulating material.

The coating material in some embodiments can reduce the miscibility of the ingredient with the encapsulating material by at least 5%, preferably 25%, more preferably at least 50%, including, 10, 15, 20, 30, 40, 60, 70, 75, 80, 85, 90, 95% or more relative to the miscibility of the ingredient that is not coated by the coating material. The coating material also may reduce the thermal sensitivity of the ingredient(s) and assist in stability of the ingredient during processing.

In some embodiments, the material used to coat the ingredient may be a water soluble and/or hydrophilic material. Non-limiting examples of suitable coating materials include, gum Arabic, cellulose, modified cellulose, gelatin, polyols (e.g., sorbitol, xylitol, maltitol), cyclodextrin, zein, polyvinylalcohol, polymethylmethacrylate, and polyurethane. Mixtures of various coating materials also may be used.

The coating thickness will vary depending on starting particle size and shape of the ingredient as well as the desired weight percent coating level. The coating thickness is preferably from about 1 to about 200 microns, including 10, 20, 30, 40, 50, 60, 70, 80, 90, 100, 110, 120, 130, 140, 150, 160, 170, 180 and 190 microns and all values and ranges there between, for example, the thickness of coating material can be from about ten to about fifty microns and twenty to 54% by weight.

In addition to providing a barrier that can reduce and/or eliminate the miscibility of the ingredient, the coating material also may have good film forming properties that facilitates the formation of a barrier between the ingredient and the encapsulating material. Film forming properties as used herein means that the coating material, after dissolution in at least one solvent (e.g., water and/or organic solvents), leaves a film on the ingredient to which it is applied, for example, once the at least one solvent evaporates, absorbs and/or dissipates on the ingredient. Furthermore, when the coating material is used in the preparation of edible compositions, such as chewing gum, the coating material can be chosen based on its taste, shelf life, stickiness, resistance to microbial growth, and other common criteria for selecting ingredients for consumption.

The ingredient can be coated with the coating material by applying the coating material to the ingredient using a pan, spray, batch, and/or continuous processes typically used to coat materials. In some embodiments, the coating material is dissolved or dispersed in a solvent to facilitate coating on the ingredient. The coating material can be delivered using conventional methods of coating substrates. In a preferred method of coating, a fluidized bed technique is employed which is described, for example, in U.S. Pat. No. 3,196,827, the relevant contents of which are incorporated herein by reference.

In a further embodiment, by coating the ingredient and encapsulating the ingredient according to the description provided herein, a longer shelf life of the edible compositions can be attained. As used herein, shelf life is an indicia of the stability of the components of the edible compositions containing the ingredient. Using flavorants and/or sweeteners for illustration, this increase in shelf life can be assessed by determining the perceived flavor and/or sweetness of the flavorant and/or sweetener contained in the edible composition. When using a coating material to coat the ingredient component a 5% increase in shelf life relative to a similar product in which the ingredient has not been coated with the barrier material can be achieved, including 10, 20, 30, 40, 50, 60, 70, 80, 90, 100% or more, as well as all values and ranges there between, increased shelf life. In another embodiment, the longer shelf life can be correlated to the time of storage after manufacture, for example at ten weeks the shelf life the edible composition containing the ingredient will demonstrate a 50%, 75%, 80%, or 90% improvement relative to a similar composition but not containing an ingredient coated with a coating material. In a further example, at twenty-four weeks of storage, the ingredient will show an 80 to 90% improvement relative to a similar composition but not containing the ingredient coated with a coating material.

For additional information regarding coating or pre-treatment of an ingredient used in a delivery system, see U.S. patent application Ser. No. 11/134,365 entitled “A Delivery System for Active Components and a Material Having Preselected Hydrophobicity as Part of an Edible Composition” and filed on May 23, 2005, the complete contents of which are incorporated herein by reference for all purposes.

### Post-treatment of an Ingredient After Encapsulation

In some embodiments, a delivery system may be post-treated prior to being added as an ingredient in an edible composition. For example, a delivery system may include a high intensity sweetener encapsulated in a first core coating comprising a low molecular weight encapsulating material (e.g., polyvinylacetate) and a second outer hydrophilic coating. This multiple coating system may provide improved resistance to the high intensity sweetener to attack from the flavor component when the delivery system is incorporated into a chewing gum, and improved stability to high temperatures. When applied to sweeteners such as aspartame, these coatings may effectuate sustained release of the sweetener, thereby extending the period of sweetener perception and enjoyment of the chewing gum or confection while at the same time enhancing the initial intensity and experience of sweetness release. The sweetener delivery system can be used in both sugar gums and in sugarless gum formulations. Examples of a post-treatment of an ingredient after encapsulation can be found in U.S. Pat. Nos. 4,933,190. For additional information regarding coating of a delivery system, see U.S. patent application Ser. No. 11/134,370 entitled “A Coated Delivery System for Active Components as Part of an Edible Composition” and filed on May 23, 2005, the complete contents of which are incorporated herein by reference for all purposes.

As one example of a hydrophilic coating, sucralose was mixed with powdered polyvinyl acetate and 5% fat and extruded at 110° C. Extensive discoloration indicating degradation of the sucralose was observed. In an alternative encapsulation, sucralose was mixed with powdered polyvinyl acetate, 2% polyvinylpyrollidone and 1% magnesium stearate and pressed into tablets at 25° C. The tablets were then heated to 80° C., which softened the polymer and fused the polyvinylacetate with the sucralose. No discoloration was observed. Thereafter, the tablets were cooled, ground and sized and analyzed. Again, no discoloration of the sucralose was observed. As another example, a polymer/sweetener matrix was prepared as described above in this paragraph. A solution of gum arabic was made and coated on the polymer/sweetener matrix particles using the method described in U.S. Pat. No. 3,196,827, the relevant portions of which are incorporated herein by reference. Coating levels were 20, 30, 40, and 50% for different samples. Chewing gums prepared with the coated polymer/sweetener matrix particles were chewed by a panel with bolus collection at 5, 10, 15, 20 minutes. Residual sucralose was analyzed in each chewed bolus. The chewing gums with higher levels of coating showed more residual sucralose remaining in the bolus at each time point.

### Multiple Layers of Encapsulation

In some embodiments, a delivery system may have multiple layers of encapsulating material for one or more ingredients. One or more of the layers may be the same or different. Each of the layers may partially or completely surround one or more ingredients or a previous encapsulation layer or form a matrix with the one or more ingredients or the previous encapsulation layer.

As one example, in a delivery system having two or more layers of encapsulation for particles of an ingredient, the delivery system may have: (1) the same inner encapsulating layer and the same inner encapsulating layer for all particles of the same ingredient; (2) the same inner encapsulating layer, but different outer encapsulating layers, for different particles of the same ingredient; (3) different inner encapsulating layers, but the same outer encapsulating layer, for different particles of the same ingredient; (4) different inner encapsulating layers and different outer encapsulating layers for different particles of the same ingredient; or (5) encapsulating layers created by different methods of encapsulation. When encapsulating layers are different, the difference may be created by, for example, different polymers, different hydrophobicities, etc.

As another example, in a delivery system having two or more layers of encapsulation for groups of multiple ingredients, the delivery system may have: (1) the same inner encapsulating layer and the same outer encapsulating layer for each group of the multiple ingredients; (2) the same inner encapsulating layer, but different outer encapsulation layers, for different groups of the multiple ingredients; (3) different inner encapsulation layers, but the same outer encapsulation layer, for different groups of the multiple ingredients; (4) different inner encapsulation layers and different outer encapsulation layers for different groups of the multiple ingredients; or (5) different layers of encapsulation for different groups of multiple ingredients created by different methods of encapsulation.

### Edible Compositions

As previously discussed above, there are many types of edible compositions that may use delivery systems or be designed for managed release of one or more ingredients. Some of these types of edible compositions are described in more detail below. The examples of edible compositions provided herein are not limiting, and are provided for illustration purposes only.

### Edible Compositions—Chewing Gum

In some embodiments, the edible composition is a chewing gum composition having a managed release of the active component. In some embodiments, the chewing gum composition comprises a chewing gum base and the delivery system(s) described herein. The delivery system(s) can be present in amounts from about 0.2% to 10% by weight based on the total weight of the chewing gum composition, including 0.5, 1.0, 2.0, 3.0, 4.0, 5.0, 6.0, 7.0, 8.0, 9.0% by weight including all values and subranges there between, for example, from about 1% to 5% by weight.

The delivery system may be incorporated with a variety of processes for preparing chewing gum compositions as known in the art. Such chewing gum compositions may include a variety of different formulations that are typically used to make chewing gum products. Typically, a chewing gum composition contains a chewable gum base portion, which is essentially free of water and is water insoluble and a water soluble bulk portion.

The water soluble portion is generally released from the gum base portion over a period of time during chewing. The gum base portion is retained in the mouth throughout the chewing. The water insoluble gum base generally comprises elastomers, elastomer solvents, plasticizers, waxes, emulsifiers, and inorganic fillers. Plastic polymers such as polyvinyl acetate, which behave somewhat as plasticizers, are also included. Other plastic polymers that may be used include polyvinyl laurate, crosslinked polyvinyl pyrrolidone and polyhydroxy alkanoates.

The elastomers may constitute from about 5% to 95% by weight of the gum base. In some embodiments, the elastomers may constitute from about 10% to 70% by weight of the gum base and in other embodiments, 15% to 45% by weight of the gum base. Examples of elastomers include synthetic elastomers such as polyisobutylene, polybutylene, isobutylene-isoprene co-polymers, styrene-butadiene co-polymers, polyvinyl acetate and the like. Elastomers may also include natural elastomers such as natural rubber as well as natural gums such as jelutong, lechi caspi, perillo, massaranduba balata, chicle, gutta hang kang or combinations thereof. Other elastomers are known to those of ordinary skill in the art.

Elastomer plasticizers may modify the finished gum firmness when used in the gum base. Elastomer plasticizers are typically present in an amount up to 75% by weight of the gum base. In some embodiments, the elastomer plasticizers are present in an amount of from about 5% to 45% by weight of the gum base and in other embodiments from about 10% to 30% by weight of gum base. Examples of elastomer plasticizers include natural rosin esters such as glycerol ester of partially hydrogenated rosin, glycerol ester of tall oil rosin, pentaerythritol esters of partially hydrogenated rosin, methyl and partially hydrogenated methyl esters of rosin, and the like. Synthetic elastomer plasticizers such as terpene resins may also be employed in gum base composition.

Waxes include synthetic and naturally occurring waxes such as polyethylene, bees wax, carnauba and the like. Petroleum waxes such a paraffin may also be used. The waxes may be present in the amount up to 30% by weight of the gum base. Waxes aid in the curing of the finished gum and help improve the release of flavor and may further extend the shelf life of the product.

Elastomer solvents are often resins such as terpene resins. Plasticizers, sometimes referred to as softeners, are typically fats and oils, including tallow, hydrogenated vegetable oils, and cocoa butter.

Gum base typically also includes a filler component. The filler component modifies the texture of the gum base and aids processing. Examples of such fillers include magnesium and aluminum silicates, clay, alumina, talc, titanium oxide, cellulose polymers, and the like. Fillers are typically present in the amount of from 1% to 60% by weight.

Emulsifiers, which sometimes also have plasticizing properties, can include glycerol monostearate, lecithin, and glycerol triacetate. Further, gum bases may also contain optional ingredients such as antioxidants, colors, and flavors.

The insoluble gum base may be present in the amount of from about 5% to 95% by weight of the chewing gum. In one embodiment, the insoluble gum base may be present in the amount of from about 10% to 50% by weight of the gum base, and in another embodiment from about 20% to 40% by weight of the gum base.

Softeners are added to the chewing gum in order to optimize the chewability and mouth feel of the gum. Softeners, also known in the art as plasticizers or plasticizing agents, are generally present in amounts from about 0.5% to 15% by weight based on the total weight of the chewing gum composition. In some embodiments, softeners can include, for example, lecithin. Further, aqueous sweetener solutions such as those containing sorbitol, hydrogenated starch hydrolysates, polyglycitols, corn syrup, and combinations thereof may be used as softeners and binding agents in the gum.

In some embodiments, chewing gum compositions may be coated or uncoated and be in the form of slabs, sticks, pellets, balls and the like. The compositions of the different forms of chewing gum will be similar but may vary with regard to the ratio of the ingredients. For example, coated gum compositions may contain a lower percentage of softeners. Pellets and balls have a small chewing gum core, which is then coated with either a sugar solution or a sugarless solution to create a hard shell. Slabs and sticks can be formulated to be softer in texture than coated chewing gum cores.

In some embodiments, the delivery system is added during the manufacture of the chewing gum composition. In another aspect of the present invention, the delivery system is added as one of the last steps, for example, the last step in the formation of the chewing gum composition.

Applicants have determined that this process modification incorporates the delivery system into the gum composition without materially binding the delivery system therein such as may occur if the delivery system is mixed directly with the gum base. Thus, the delivery system, while only loosely contained within the gum composition can more effectively release the active component therefrom during a typical chewing occasion. Thus, a material portion of the delivery system is free of the gum base and the corresponding ingredients of the chewing gum.

Coating techniques for applying a coating for a chewing gum composition such as pan and spray coating are well known. In one embodiment, coating with solutions adapted to build a hard candy layer can be employed. Both sugar and sugar free sugar alcohols may be used for this purpose together with high intensity sweeteners, colorants, flavorants and binders.

Other components may be added in minor amounts to the coating syrup and can include, but are not limited to, moisture absorbing compounds, anti-adherent compounds, dispersing agents and film forming agents. The moisture absorbing compounds suitable for use in the coating syrups include mannitol or dicalcium phosphate. Examples of useful anti-adherent compounds, which may also function as fillers, can include talc, magnesium trisilicate and calcium carbonate. These ingredients may be employed in amounts of from about 0.5% to 5% by weight of the syrup. Examples of dispersing agents, which may be employed in the coating syrup, include titanium dioxide, talc or other anti-adherent compounds as set forth above.

The coating syrup can be heated and a portion thereof deposited on the cores. Usually a single deposition of the coating syrup is not sufficient to provide the desired amount or thickness of coating and second, third or more coats of the coating syrup may be applied to build up the weight and thickness of the coating to desired levels with layers allowed to dry in-between coats.

Examples of coating methods, apparatus, and compositions are also included in U.S. Pat. Nos. 6,783,783 to Clark et al., 6,689,417 to Brandt et al., 6,638,550 to Banko, 5,087,460 to Cherukuri, 5,023,093 to Cherukuri, and 4,840,797 to Boursier which are incorporated in their entirety herein for all purposes.

In some embodiments, a method of preparing a chewing gum composition can include sequentially adding the various chewing gum ingredients including the delivery system of the present invention to any commercially available mixer known in the art that will suitably mix the ingredients. After the ingredients have been thoroughly mixed, the gum base can be discharged from the mixer and shaped into the desired form such as by rolling into sheets and cutting into sticks, extruding into chunks, or casing into pellets.

Generally, the ingredients are mixed by first melting the gum base and adding it to a mixer. The base may also be melted or softened/warmed in the mixer itself. Colors or emulsifiers may also be added at this time. A softener may be added to the mixer at this time, along with syrup and a portion of the bulking agent. Further additions of the bulking agent are then added to the mixer. Flavorants are typically added with the final portion of the bulking agent. Finally, the delivery system exhibiting a predetermined tensile strength is added to the resulting mixture. Other optional ingredients are added in the batch in a typical fashion, well known to those of ordinary skill in the art.

In some embodiments, a batch or kettle mixer may be used to make some or all of the chewing gum ingredients. In some embodiments, an extruder or other continuous type mixer may be used to make some or all of the chewing gum ingredients. Different ingredients may be added at different times and/or points in the mixing process to create the desired result in the chewing gum.

Examples of gum mixing can be found in U.S. Pat. Nos. 6,858,237, 6,811,797, 6,440,472, 6,086,925, 6,030,647, 6017,565, 5,976,581, 5,908,645, 5,827,549, 5,800,847, 5,614,234, 5,612,071, 5,545,416, 5,543,160, 5,192,562, 5,045,325, 4,940,594, 4,737,366, 4,579,738, 4,555,407, 2,256,190, and 947,635, the contents of all of which are incorporated herein by reference for all purposes.

In some embodiments, the entire mixing procedure can take from five to fifteen minutes, but longer mixing times may be required or desired. Those skilled in the art will recognize that many variations of the above-described procedure may be followed.

After the ingredients are mixed, the gum mass may be formed into a variety of shapes and products. For example, the ingredients may be formed into pellets or balls and used as cores to make a coated chewing gum product. However, any type of chewing gum product can be utilized with the delivery systems.

In some embodiments, a chewing gum also may include a liquid or other center-fill type material. Examples of center-fill chewing gums and other products and methods for making center-fill chewing gums and other products can be found in U.S. Pat. Nos. 6,652,839, 6,623,266, 6,558,727, 6,491,540, 6,472,001, 6,284,291, 6,280,780, 6,280,762, 5,612,070, 5,498,429, 5,125,819, 4,980,178, 4,975,288, 4,938,128, 4,683,138, 4,642,235, 4,513,012, 4,466,983, 4,316,915, 4,301,178, 4,292,329, 4,252,829, 4,157,402, 4,156,740, 3,894,154, 3,857,963, 3,806,290, and 810,210, as well as U.S. patent application Ser. No. 10/925,822, the contents of all of which are incorporated herein by reference for all purposes.

If a coated product is desired, the coating may be a hard or soft coating and may contain ingredients such as flavorants, sensates, artificial sweeteners, dispersing agents, coloring agents, film formers and binding agents. Flavorants contemplated by the present invention, can include those commonly known in the art such as essential oils, synthetic flavors, or mixtures thereof, including but are not limited to, oils derived from plants and fruits such as citrus oils, fruit essences, peppermint oil, spearmint oil, other mint oils, clove oil, oil of wintergreen, anise and the like. In some embodiments, the flavorants may also be added to the coating syrup in an amount from about 0.2% to 1.2%. In another embodiment, the coating may be present in amounts, and more preferably from about 0.7% to 1.0%. In some embodiments, sensates may also be added to the coating syrup in an amount from about 0.0001 to 1.5%.

Dispersing agents are often added to syrup coatings for the purpose of whitening and tack reduction. Dispersing agents contemplated by the present invention to be employed in the coating syrup can include titanium dioxide, talc, or any other anti-stick compound. The dispersing agent may be added to the coating syrup in an amount such that the coating contains from about 0.1% to 1.0%, including 0.2, 0.3, 0.4, 0.5, 0.6, 0.7, 0.8, 0.9 and all values and ranges there between, for example, from about 0.3% to 0.6% by weight of the agent.

Coloring agents may be added directly to the coating syrup in dye or lake form. Coloring agents contemplated by the present invention can include food quality dyes and lakes. Film formers may be added to the coating syrup including methylcellulose, carboxymethyl cellulose, ethyl cellulose, hydroxyethyl cellulose, and the like or combinations thereof. Binding agents may be added either as an initial coating on the chewing gum center or may be added directly to the coating syrup. Binding agents contemplated by the present invention can include gum arabic, hydrolyzed indigestible starches, gum talha, gelatin, vegetable gums, and the like. The binding agents, when added to the coating syrup, are typically added in amounts from about 0.5% to 10% by weight.

### Edible Compositions—Compressible Chewing Gum

The gum base used in the compressible chewing gum compositions of the present invention may be any conventional chewing gum base used in making chewing gum. As opposed to molten, or thermoplastic, gum base, however, the gum base in the compressible chewing gum compositions may be in a particulate form, such as, but not limited to, a powdered or granular gum base. The particulate gum base may be essentially free of water and can readily be formed into any desired shape, such as by compression.

The gum base may include any component known in the chewing gum art. For example, the gum base may include elastomers, bulking agents, waxes, elastomer solvents, emulsifiers, plasticizers, fillers, and mixtures thereof.

The elastomers (rubbers) employed in the gum base may vary depending upon various factors such as the type of gum base desired, the consistency of gum composition desired and the other components used in the composition to make the final chewing gum product. The elastomer may be any water-insoluble polymer known in the art, and includes those gum polymers utilized for chewing gums and bubble gums. Illustrative examples of suitable polymers in gum bases include both natural and synthetic elastomers. For example, those polymers which are suitable in gum base compositions include, without limitation, natural substances (of vegetable origin) such as chicle, natural rubber, crown gum, nispero, rosidinha, jelutong, perillo, niger gutta, tunu, balata, guttapercha, lechi capsi, sorva, gutta kay, and the like, and mixtures thereof. Examples of synthetic elastomers include, without limitation, styrene-butadiene copolymers (SBR), polyisobutylene, isobutylene-isoprene copolymers, polyethylene, polyvinyl acetate and the like, and mixtures thereof.

The amount of elastomer employed in the gum base may vary depending upon various factors such as the type of gum base used, the consistency of the gum composition desired and the other components used in the composition to make the final chewing gum product. In general, the elastomer will be present in the gum base in an amount from about 10% to about 80% by weight, desirably from about 35% to about 40% by weight.

In some embodiments, the gum base may include wax which can soften the polymeric elastomer mixture and can improve the elasticity of the gum base. When present, the waxes employed will have a melting point below about 60° C., and preferably between about 45° C. and about 55° C. The low melting wax may be a paraffin wax. The wax may be present in the gum base in an amount from about 6% to about 10%, and preferably from about 7% to about 9.5%, by weight of the gum base.

In addition to the low melting point waxes, waxes having a higher melting point may be used in the gum base in amounts up to about 5%, by weight of the gum base. Such high melting waxes include beeswax, vegetable wax, candelilla wax, carnuba wax, most petroleum waxes, and the like, and mixtures thereof.

In addition to the components set out above, the gum base may include a variety of other ingredients, such as components selected from elastomer solvents, emulsifiers, plasticizers, fillers, and mixtures thereof.

The gum base may contain elastomer solvents to aid in softening the elastomer component. Such elastomer solvents may include those elastomer solvents known in the art, for example, terpinene resins such as polymers of alpha-pinene or beta-pinene, methyl, glycerol and pentaerythritol esters of rosins and modified rosins and gums such as hydrogenated, dimerized and polymerized rosins, and mixtures thereof. Examples of elastomer solvents suitable for use herein may include the pentaerythritol ester of partially hydrogenated wood and gum rosin, the pentaerythritol ester of wood and gum rosin, the glycerol ester of wood rosin, the glycerol ester of partially dimerized wood and gum rosin, the glycerol ester of polymerized wood and gum rosin, the glycerol ester of tall oil rosin, the glycerol ester of wood and gum rosin and the partially hydrogenated wood and gum rosin and the partially hydrogenated methyl ester of wood and rosin, and the like, and mixtures thereof. The elastomer solvent may be employed in the gum base in amounts from about 2% to about 15%, and preferably from about 7% to about 11%, by weight of the gum base.

The gum base may also include emulsifiers which aid in dispersing the immiscible components into a single stable system. Useful emulsifiers can include, but are not limited to, glyceryl monostearate, lecithin, fatty acid monoglycerides, diglycerides, propylene glycol monostearate, and the like, and mixtures thereof. The emulsifier may be employed in amounts from about 2% to about 15%, and more specifically, from about 7% to about 11%, by weight of the gum base.

The gum base may also include plasticizers or softeners to provide a variety of desirable textures and consistency properties. Because of the low molecular weight of these ingredients, the plasticizers and softeners are able to penetrate the fundamental structure of the gum base making it plastic and less viscous. Useful plasticizers and softeners can include lanolin, palmitic acid, oleic acid, stearic acid, sodium stearate, potassium stearate, glyceryl triacetate, glyceryl lecithin, glyceryl monostearate, propylene glycol monostearate, acetylated monoglyceride, glycerine, and the like, and mixtures thereof. Waxes, for example, natural and synthetic waxes, hydrogenated vegetable oils, petroleum waxes such as polyurethane waxes, polyethylene waxes, paraffin waxes, microcrystalline waxes, fatty waxes, sorbitan monostearate, tallow, propylene glycol, mixtures thereof, and the like, may also be incorporated into the gum base. The plasticizers and softeners are generally employed in the gum base in amounts up to about 20% by weight of the gum base, and more specifically in amounts from about 9% to about 17%, by weight of the gum base.

Plasticizers also include hydrogenated vegetable oils, such as soybean oil and cottonseed oils, which may be employed alone or in combination. These plasticizers provide the gum base with good texture and soft chew characteristics. These plasticizers and softeners are generally employed in amounts from about 5% to about 14%, and more specifically in amounts from about 5% to about 13.5%, by weight of the gum base.

Anhydrous glycerin may also be employed as a softening agent, such as the commercially available United States Pharmacopeia (USP) grade. Glycerin is a syrupy liquid with a sweet warn taste and has a sweetness of about 60% of that of cane sugar. Because glycerin is hygroscopic, the anhydrous glycerin may be maintained under anhydrous conditions throughout the preparation of the compressible chewing gum composition.

In some embodiments, the gum base of the compressible chewing gum composition may also include effective amounts of bulking agents such as mineral adjuvants which may serve as fillers and textural agents. Useful mineral adjuvants can include calcium carbonate, magnesium carbonate, alumina, aluminum hydroxide, aluminum silicate, talc, tricalcium phosphate, dicalcium phosphate, calcium sulfate and the like, and mixtures thereof. These fillers or adjuvants may be used in the gum base compositions in various amounts. Preferably the amount of filler, when used, will be present in an amount from about 15% to about 40%, and desirably from about 20% to about 30%, by weight of the gum base.

A variety of traditional ingredients may be optionally included in the gum base in effective amounts such as flavor agents and coloring agents, antioxidants, preservatives, and the like. For example, titanium dioxide and other dyes suitable for food, drug and cosmetic applications, known as F. D. & C. dyes, may be utilized. An anti-oxidant such as butylated hydroxytoluene (BHT), butylated hydroxyanisole (BHA), propyl gallate, vitamin E and mixtures thereof, may also be included. Other conventional chewing gum additives known to one having ordinary skill in the chewing gum art may also be used in the gum base.

The compressible chewing gum compositions may include amounts of conventional additives selected from the group consisting of sweetening agents, plasticizers, softeners, emulsifiers, waxes, fillers, bulking agents (carriers, extenders, bulk sweeteners), mineral adjuvants, flavor agents and coloring agents, antioxidants, acidulants, thickeners, medicaments, and the like, and mixtures thereof. Some of these additives may serve more than one purpose. For example, in sugarless gum compositions, a sweetener, such as maltitol or other sugar alcohol, may also function as a bulking agent or sensate.

Bulk sweeteners, such as sugars, sugarless bulk sweeteners, or the like, or mixtures thereof, generally can be present in amounts of about 5% to about 95% by weight of the chewing gum composition.

Suitable sugar sweeteners can generally include mono-saccharides, di-saccharides and poly-saccharides such as but not limited to, sucrose (sugar), dextrose, maltose, dextrin, xylose, ribose, glucose, mannose, galactose, fructose (levulose), invert sugar, fructo oligo saccharide syrups, partially hydrolyzed starch, corn syrup solids and mixtures thereof.

Suitable sugarless bulk sweeteners can include sugar alcohols (or polyols) such as, but not limited to, sorbitol, xylitol, mannitol, galactitol, maltitol, hydrogenated isomaltulose (ISOMALT™), lactitol, erythritol, hydrogenated starch hydrolysates, stevia and mixtures thereof.

Suitable hydrogenated starch hydrolysates can include those disclosed in U.S. Pat. Nos. 25,959, 3,356,811, 4,279,931 and various hydrogenated glucose syrups and/or powders which contain sorbitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenated starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN™, a commercially available product manufactured by Roquette Freres of France, and HYSTAR™, a commercially available product manufactured by Lonza, Inc., of Fairlawn, N.J., can also be useful.

The plasticizers, softening agents, mineral adjuvants, waxes and antioxidants discussed above, as being suitable for use in the gum base, may also be used in the compressible chewing gum composition. Examples of other conventional additives which may be used include emulsifiers, such as lecithin and glyceryl monostearate, thickeners, used alone or in combination with other softeners, such as methyl cellulose, alginates, carrageenan, xanthan gum, gelatin, carob, tragacanth, locust bean, and carboxy methyl cellulose, acidulants such as malic acid, adipic acid, citric acid, tartaric acid, fumaric acid, and mixtures thereof, and fillers, such as those discussed above under the category of mineral adjuvants.

Other conventional gum additives known to one having ordinary skill in the chewing gum art also may be used in the compressible chewing gum compositions.

The particulate gum base may be formed using standard grinding techniques known in the art. The starting material may be any conventional gum base, such as those used to produce molten gum bases. The particulate gum base may be formed, for example, by shredding, grinding or crushing the gum base or other processes, as described in U.S. Pat. Nos. 3,262,784, 4,405,647, 4,753,805 and 6,290,985 and U.S. Publication No. 2003/00276871, all of which are incorporated herein by reference in their entirety.

Desirably, the particulate gum base is ground or the like into a particulate form that is similar in particle size to the tableting powder. By using components of like particle size, a homogenous mix of gum base and tableting powder may be achieved, which may provide a gum tablet of similar homogenous make-up. The gum base and tableting powder may have a particle size of about 4 to about 100 mesh, desirably about 8 to about 25 mesh, and more desirably about 12 to about 20 mesh.

The particulate gum base may be present in amounts of about 10% to about 80% by weight of the chewing gum composition, or tablet, desirably about 20% to about 50% by weight, and more desirably about 30% to about 40% by weight.

The particulate gum base may be combined with a tableting powder to form the pressed gum tablet. The tableting powder can be in a dry, finely-divided form. Desirable particle size is provided above. The tableting powder may be a sucrose-based, dextrose-based or polyol-based powder, or combinations thereof. For example, the polyol-based powder may be a sorbitol or mannitol powder. The tableting powder may include other optional ingredients, such as flavor agents, color agents, sugar and/or sugarless sweeteners, and the like and combinations thereof.

In some embodiments, it may be desirable to combine a food-grade lubricant with the particulate gum base and tableting powder. Food-grade lubricants may assist in processing the gum composition into pressed tablets. More specifically, lubricants are used to prevent excess wear on dies and punches in tableting manufacture. Lubricants may be useful immediately after compression of the tablet within the die to reduce friction between the tablet and inner die wall.

The food-grade lubricant may be added separately or it may be included with the tableting powder, as in some commercially available tableting powders. Examples of suitable food-grade lubricants include: metallic stearates; fatty acids; hydrogenated vegetable oil; partially hydrogenated vegetable oils; animal fats; polyethylene glycols; polyoxyethylene monostearate; talc; silicon dioxide; and combinations thereof. Food-grade lubricants may be present in amounts of about 0-6% by weight of the gum composition.

As described above, the compressible chewing gum composition can be in the form of a pressed gum tablet. In some embodiments, the particulate gum base and modified release ingredients are pressed into a tablet form. Upon chewing, the pressed gum tablet consolidates into a soft chewy substance.

In some embodiments, the compressible chewing gum composition is a single-layer pressed tablet. In some embodiments, the compressible chewing gum composition is a multi-layer pressed tablet. Multi-layer tablet embodiments may have any desirable number of layers. Different layers may have the same or different thicknesses. In addition, different layers may include the same or different ingredients.

The pressed gum tablet also may have a coating layer surrounding the tablet. The coating layer may contain any ingredients conventionally used in the chewing gum art. For instance, the coating may contain sugar, polyols or high intensity sweeteners or the like, coloring agents, flavor agents and warming and/or cooling agents, among others. In some embodiments, the coating layer also may include a modified release ingredient as described above.

The compressible chewing gum compositions, or pressed tablets, desirably have a very low moisture content. In some embodiments, the tablets are essentially free of water. Accordingly, some embodiments have a total water content of greater than about 0% to about 5% by weight of the composition. The density of the composition, or tablet, may be about 0.2 to about 0.8 g/cc. Further, the compressible chewing gum compositions, or tablets, may have a dissolution rate of about 1 to about 20 minutes. When in a pressed tablet form, the chewing gum may have a Shore hardness of about 30 to about 200.

In contrast to dough mixed chewing gums where the gum mixture can achieve temperatures of 35 C to 60 C, compressed chewing gum temperatures can remain around ambient temperature (23 C to 25 C). In some embodiments, subjecting the compressible chewing gum compositions to lower temperatures can protect temperature sensitive ingredients from thermal degradation. Similarly, the absence of intimate mixing at temperatures above ambient can protect delivery systems that include temperature sensitive ingredients or ingredients subject to degradation from gum ingredients such as flavors, plasticizers, etc. Thus, ingredients susceptible to thermal or chemical degradation due to conventional dough mixing can be less likely to experience degradation in compressed chewing gum systems.

In some embodiments, methods of preparing pressed chewing gum tablets are employed. In accordance therewith, a particulate chewing gum base is provided. The particulate chewing gum base may be prepared by grinding or other similar means to obtain the desired particulate form, such as, for example, a finely divided powder. The particulate chewing gum base is mixed with a tableting powder, as described above. The particulate gum base and tableting powder may be mixed in any conventional way.

It may be desirable to mix the particulate gum base and tableting powder until a homogenous mix is achieved. Further, it may be desirable to use a particulate gum base and tableting powder that have similarly sized particles to obtain such a homogenous mixture. A homogenous mixture may provide a pressed gum tablet of similar homogenous make-up. Conventional mixing apparatus known to those skilled in the art may be used.

A modified release ingredient may be added to the mixture of particulate gum base and tableting powder during mixing. Once the modified release ingredients and any other components are blended in, the mixture may be passed through a screen of desired mesh size. Other components, such as lubricants, may be added and the batch may be further mixed. It may be desirable to mix until the batch is a homogenous powder. The batch then may be punched or pressed into gum tablets on a conventional tableting machine, such as a Piccola Model D-8 mini rotary tablet press or a Stokes machine.

Alternatively, the compressible chewing gum composition can be prepared by forming a dough mixed chewing gum composition and granulating the mixture using any suitable granulation process. The granulated mixture may be passed through a screen of desired mesh size. The modified release ingredient(s) may be added to the granulated mixture and mixed. Other components, such as lubricants, may be added and the batch may be further mixed. It may be desirable to mix until the batch is a homogenous powder. The batch then may be punched or pressed into gum tablets on a conventional tableting machine, such as a Piccola Model D-8 mini rotary tablet press or a Stokes machine.

In single-layer embodiments, the powder batch may be pressed into gum tablets as described above.

In multi-layer embodiments, a separate layer batches may be filled into the tableting machine in sequence and pressed together to form a multi-layer gum tablet.

Any number of powder batches may be filled into the tableting machine in any sequence and compressed together to form tablets having any desired number of layers.

### Edible Compositions—Hard Boiled Confectionery

In some embodiments, particularly lozenges or hard candies, the delivery system, which can be dispersed in a glassy polymer matrix, may be present in the composition in amounts of about 0.001% to about 10% by weight of the composition, more desirably about 0.001% to about 5% by weight.

In some embodiments, confectionery compositions can be produced by batch processes. Such confections may be prepared using conventional apparatus such as fire cookers, cooking extruders, and/or vacuum cookers.

In some embodiments, the bulk sweetener (sugar or sugar free) and a solvent (e.g., water), are combined in a mixing vessel to form a slurry. The slurry is heated to about 70° C. to 120° C. to dissolve any sweetener crystals or particles and to form an aqueous solution. Once dissolved, heat and vacuum are applied to cook the batch and boil off water until a residual moisture of less than about 4% is achieved. The batch changes from a crystalline to an amorphous, or glassy, phase. The delivery system(s) can then admixed in the batch by mechanical mixing operations, along with any other optional additives, such as coloring agents, flavorants, and the like. The batch is then cooled to about 50° C. to 10° C. to attain a semi-solid or plastic-like consistency.

The optimum mixing required to uniformly mix the delivery system(s), flavors, colorants and other additives during manufacturing of hard confectionery is determined by the time needed to obtain a uniform distribution of the materials. Normally, mixing times of from four to ten minutes have been found to be acceptable.

Once the candy mass has been properly tempered, it may be cut into workable portions or formed into desired shapes having the correct weight and dimensions. A variety of forming techniques may be utilized depending upon the shape and size of the final product desired. Once the desired shapes are formed, cool air is applied to allow the comestibles to set uniformly, after which they are wrapped and packaged.

Alternatively, various continuous cooking processes utilizing thin film evaporators and injection ports for incorporation of ingredients including the delivery system(s) are known in the art and can be used as well.

The apparatus useful in accordance with the present invention comprise cooking and mixing apparatus well known in the confectionery manufacturing arts, and selection of specific apparatus will be apparent to one skilled in the art.

### Edible Compositions—Pressed Tablets

In some embodiments, confectionery compositions in the form of pressed tablets such as mints may can be made by combining finely sifted sugar or sugar substitute, flavoring agent (e.g. peppermint flavor) binding agent such as gum arabic, and an optional coloring agent. The flavoring agent, binding agent are combined and then gradually the sugar or sugar substitute are added along with a coloring agent if needed.

The product is then granulated by passing through a sieve of desired mesh size (e.g., 12 mesh) and then dried typically at temperatures of from about 55° C. to 60° C. The resulting powder is fed into a tableting machine fitted with a punch and the resulting pellets are broken into granules and then pressed.

### Edible Compositions—Thin Films

In some embodiments, edible films that dissolve in the oral cavity can be used. Such films are made from various hydrocolloids including pullulan, starches, alginates, and combinations thereof.

In some embodiments, film-forming agents can include, but are not limited to, pullulan, hydroxypropylmethyl cellulose, hydroxyethyl cellulose, hydroxypropyl cellulose, polyvinyl pyrrolidone, methyl cellulose carboxymethyl cellulose, polyvinyl alcohol, polyethylene glycol, polyacrylic acid, methylmethacrylate copolymer, carboxyvinyl polymer, amylase, high amylase starch, hydroxypropylated high amylase starch, chemically modified starch, dextrin, indigestible dextrin, chitin, chitosan, levan, elsinan, collagen, zein, gluten, soy protein isolate, whey protein isolate, casein, and mixtures thereof. In some embodiments, other film forming agents can include hydrocolloids such as natural seaweeds, natural seed gum, natural plant exudates, natural fiber extracts, biosynthetic gums, gelatins, biosynthetic process starch or cellulosic materials, alginates, sodium alginate, calcium alginate, carrageenans, guar gum, locust gum, tara gum, gum arabic, acacia gum, ghatti gum, agar gum, xanthan gum, pectin, tragacanth gum, and combinations thereof. It will be readily recognized by those skilled in the art that these materials can serve more than one function in the invention. For example, in addition to film forming properties, many of these materials also function as binders, thickeners, and stabilizers.

In some embodiments, the edible film is prepared from an aqueous dispersion or solution of film forming agents. Film forming agents can include materials with affinities for water such that they swell and increase viscosity when introduced into water. In some embodiments, edible films are formed by introducing the film forming agents into water with mixing to prevent clumping. In some embodiments, other ingredients such as the delivery system(s) are incorporated into the dispersion or solution either before or after the addition of the film forming agents. After a homogeneous mixture of film forming agents, delivery system(s) and other additives such as sweeteners, flavors, and plasticizers is achieved, the solution is heated to remove excess moisture and cast upon a substrate for drying. The resulting matrix includes a structure including long and short chain polymers some of which have linear conformations and some of which have branched conformations. Upon drying, the edible film is formed and can be cut and/or packaged.

### Edible Compositions—Soft Confectionery

In some embodiments, the delivery system can be used in various soft confectionery formats. Soft confectionery formats can include nougat, caramel, taffy, gummies, and jellies.

In some embodiments, a nougat composition can include two principal components, a high boiled candy and a frappe. By way of example, egg albumen or substitute thereof is combined with water and whisked to form a light foam. Sugar and glucose are added to water and boiled typically at temperatures of from about 130° C. to 140° C. and the resulting boiled product is poured into a mixing machine and beaten until creamy. The beaten albumen and flavoring agent are combined with the creamy product and the combination is thereafter thoroughly mixed.

In some embodiments, a caramel composition can include sugar (or sugar substitute), corn syrup (or polyol syrup), partially hydrogenated fat, milk solids, water, butter, flavors, emulsifiers, and salt. To prepare the caramel, the sugar/sugar substitute, corn syrup/polyol syrup, and water can be mixed together and dissolved over heat. Then, the milk solids can be mixed in to the mass to form a homogeneous mixture. Next, the minor ingredients can be mixed in with low heat. The heat can then be increased to boiling. Once sufficient water is removed and color/flavor developed, the mass can be cooled somewhat and temperature sensitive ingredients (including the delivery system(s)) can be mixed in prior to discharging and forming/shaping/wrapping the finished product.

In some embodiments, a taffy composition can include sugar (or sugar substitute), corn syrup (or polyol syrup), partially hydrogenated fat, water, flavors, emulsifiers, and salt. The process for preparing taffy can be similar to that for caramel and, optionally, the final taffy mass can be pulled to develop its desired texture.

In some embodiments, a gummi composition can include sugar (or sugar substitute), corn syrup (or polyol syrup), gelatin (or suitable hydrocolloid), flavor, color, and optionally acid. The gummi composition can be prepared by hydrating the gelatin or suitable hydrocolloid, heating the sugar/corn syrup (sugar substitute/polyol syrup) and combining the two components with heat. Once the combined mixture reaches its final temperature or suitable sugar solids level, minor components such as flavor, color, the delivery system(s), etc. can be incorporated into the mixture and then poured into molds prior to cooling, wrapping, and finishing. Various surface treatments such as applications of wax or fat can be applied to decrease sticking.

In some embodiments, a jelly composition can include a starch-based jelly or a pectin-based jelly. As with gummis, jelly products can be produced by hydrating the hydrocolloid and combining the hydrated mixture with a cooked syrup component. The mixture can then be cooked to a final moisture content and minor components can be incorporated (including the delivery system(s)). As with gummis, jelly candies can be poured into molds such as starch molds. As with gummis, surface treatments such as fats or waxes can be applied. Additionally, jelly candies can have dry surface treatments such as applications of sanding sugar, acid, non-pareils, and the like.

### Edible Compositions—Spun Sugar/Sugar Substitute

In some embodiments, an edible composition can be made by subjecting a carbohydrate matrix to melt spinning. Melt spinning is a process well known in the art of non-woven fiber and fabric manufacture. In melt spinning, a solid, polymeric material is melted to form a viscous liquid. This viscous liquid is then forced through a die with multiple small holes (also known as a spinneret) to form multiple filaments. The filaments then solidify upon cooling into fibers that can, depending on the polymeric starting material, be stretched to add strength. The fibers can then be further processed to form fabrics such as nylon, saran, and polyester. When a carbohydrate matrix forms the feedstock for the melt spinning process, a cotton-candy like web results. In some embodiments, the cotton candy like web can have delivery system(s) incorporated into it. Additionally, the cotton candy like web can be formed into discrete dosage units that resemble typical dosage forms such as tablets.

Melt spinning can be accomplished by any means well known in the art. In some embodiments, a cotton candy machine such as the Econo-Floss Model 3017 manufactured by Gold Medal Products Co., Cincinnati, Ohio is used. Any other apparatus or physical process which provides similar forces and temperature gradients can also be used.

Following melt spinning, the web can be manipulated to form a sheet. Manipulation can include, but is not limited to pulling, twisting, or entangling. In some embodiments, the manipulated sheet can then be formed into discrete dosage units. As used herein, the term “discrete dosage unit” refers to any format, such as tablets, discs, or lozenges, of the melt-spun web or dosage delivery vehicle that provides an end-user with an intended benefit. Discrete dosage units can be produced using conventional forming equipment following manipulation of the web or dosage delivery vehicle into a sheet. In some embodiments, the discrete dosage unit can be formed by passing the manipulated sheet of web or dosage delivery vehicle through a shaping roller to form a dosage unit in a conventional confectionery shape.

### Edible Compositions—Chocolate Confectionery

Chocolate processing begins with sorting, cleaning, and then roasting the cocoa seeds or beans. The seeds are then cracked to produce seed bits known as nibs. The nibs then undergo a sizing processing called winnowing. After winnowing, the nibs are then milled to form a thick paste known as chocolate liquor. The chocolate liquor can then be pressed to separate out cocoa butter with the remaining material being sized into chocolate powder. Chocolate crumb is formed by mixing chocolate liquor with condensed milk, cocoa fat, and sugar. Chocolate crumb becomes finished chocolate by refining through rollers followed by conching and tempering. Chocolate candies can be formed by mixing other ingredients such as caramel or nuts and other inclusions and forming the mass into finished product shapes which are packaged for sale. Many configurations and combinations are known to those in the art. The delivery system(s) can be added at any suitable point in the process. In addition to chocolate that meets the standard of identity for labeling the product as chocolate, in some embodiments, cocoa or chocolate containing compositions such as compound coatings and the like can be produced and can have the delivery system(s) added to them.

Further details regarding the preparation of confectionery compositions can be found in Skuse's Complete Confectioner (13^{th} Edition) (1957) including pp. 41-71, 133-144, and 255-262; and Sugar Confectionery Manufacture (2^{nd} Edition) (1995), E. B. Jackson, Editor, pp. 129-168, 169-188, 189-216, 218-234, and 236-258 each of which is incorporated herein by reference for all purposes.

### Introduction of Examples

The following examples provide examples of delivery systems and chewing gums that contain one or more delivery systems as an ingredient. The delivery systems that include an ingredient encapsulated with an encapsulating material provide for delayed release of the ingredient. Other modifications to the delivery systems and/or chewing gums can be made in accordance with the methods and techniques described herein.

While specific examples are provided and discussed below, the ranges and combinations of ingredients provided in the examples are not limiting and are for illustration purposes only. Other ranges or combinations also may be possible and are contemplated herein. For example, in some embodiments the gum base may be a range of 20-45% by weight of the gum compositions.

In some embodiments, the chewing gum examples described herein may be made using a conventional batch or continuous gum making process wherein a delivery system is added as one of the gum ingredients. For example, gum base may be melted in a mixer and the remaining ingredients added to the gum base. The melted gum base and other ingredients are mixed to disperse the ingredients and create a homogenous mass. The resulting gum mixture can be cooled and then sized and conditioned if necessary prior to packaging. The gum mixture also may be scored or otherwise formed into a desired shape (e.g., pellet, slab, stick, ball) as part of or in addition to the gum mixing process.

In some embodiments of the chewing gum examples provided herein, the tensile strength for the delivery system(s) used in the chewing gum may be 6500 or greater (e.g., greater than 10000). In addition, the delivery system may include at least one polymer having a water absorption of about 0.01% to 50% by weight. In some embodiments, the polymer may have a water absorption of about 0.1% to about 15% by weight.

In some embodiments, if not already included, one or more additional ingredients (e.g., 0-2% sensate, 0-3% acidulants, 0-1% hydrogenated starch hydrolysates or other humectant, 0-5% high intensity sweeteners, 0-1% lecithin, or one or more other ingredients described herein) also may be included in the example chewing gum compositions. Alternatively, or in addition, in some embodiments, the chewing gum may include 0-5% of a non-encapsulated ingredient, such as the same ingredient included in a delivery system added to the chewing gum. Also, in some embodiments, the gums in the examples may include one or more coatings.

In some embodiments, if not already included, the chewing gum examples described herein may include 0-3% of an ingredient already added to the chewing gum via a first delivery system in a second delivery system, wherein the encapsulating material used for the first delivery system (e.g., polyvinylacetate) is different than that encapsulating material used for the second delivery system (e.g., EVA), the encapsulating material used in the first delivery system has a different hydrophobicity than the encapsulating material used in the second delivery system, and/or the first delivery system includes one or more tensile strength modifying agents that are not present in the second delivery system. In some embodiments, the chewing gum examples described herein may include different particle sizes of ingredients and/or delivery systems.

The following co-pending applications all relate to oral delivery systems and are incorporated herein by reference in their entirety: U.S. patent application Ser. No. 11/083,968 entitled “A Delivery System for Active Component as Part of an Edible Composition Having Preselected Tensile Strength” and filed on Mar. 21, 2005; U.S. patent application Ser. No. 10/719,298 entitled “A Delivery System for Active Components as Part of an Edible Composition” and filed on Nov. 21, 2003; International Application No. PCT/US04/37185 and filed on Nov. 22, 2004; U.S. patent application Ser. No. 11/135,149 entitled “Enhanced Flavor Release Comestible Compositions and Methods for Same” and filed on May 23, 2005; U.S. patent application Ser. No. 11/135,153 entitled “Controlled Release Oral Delivery System” and filed on May 23, 2005; U.S. patent application Ser. No. 11/134,367 entitled “A Delivery System for Active Components as Part of an Edible Composition” and filed on May 23, 2005; U.S. patent application Ser. No. 11/134,370 entitled “A Coated Delivery System for Active Components as Part of an Edible Composition” and filed on May 23, 2005; U.S. patent application Ser. No. 11/134,356 entitled “An Edible Composition Including a Delivery System for Active Components” and filed on May 23, 2005; U.S. Patent application Ser. No. 11/134,371 entitled “A Delivery System for Active Components as Part of an Edible Composition Including a Ratio of Encapsulating Material and Active Component” and filed on May 23, 2005; U.S. patent application Ser. No. 11/134,480 entitled “A Delivery System for Active Components as Part of an Edible Composition Having Selected Particle Size” and filed on May 23, 2005; U.S. patent application Ser. No. 11/134,369 entitled “A Compressed Delivery System for Active Components as Part of an Edible Composition” and filed on May 23, 2005; U.S. patent application Ser. No. 11/134,365 entitled “A Delivery System for Active Components and a Material Having Preselected Hydrophobicity as Part of an Edible Composition” and filed on May 23, 2005; and U.S. patent application Ser. No. 11/134,364 entitled “A Delivery System for Coated Active Components as Part of an Edible Composition” and filed on May 23, 2005.

### EXAMPLES

Examples of various encapsulations of one or more ingredients to form delivery systems and edible compositions that contain such delivery systems are provided herein.

### Ingredient Examples

### Ingredient Examples of Single Ingredients in a Delivery System

### Example 1

### Encapsulation of Glycyrrhizin—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Glycyrrhizin | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Glycyrrhizin is then added to the resulting mixture and mixed under high shear to completely disperse the ingredients. The resulting filled polymer melt is cooled and ground to produce a powdered material with a particle size of less than 420 microns. The encapsulated Glycyrrhizin matrix is stored in air tight containers with low humidity below 35° C.

### Example 2

### Encapsulation of Xylitol—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Xylitol | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Xylitol is then added to the resulting mixture and mixed under high shear to completely disperse the ingredients. The resulting filled polymer melt is cooled and ground to produce a powdered material with a particle size of less than 420 microns. The encapsulated xylitol matrix is stored in air tight containers with low humidity below 35° C.

### Example 3

### Encapsulation of Erythritol

Composition:

| Ingredient | Weight percent |
|---|---|
| Polyvinyl Acetate | 55.00% |
| Hydrogenated Oil | 3.75% |
| Glycerol Monostearate | 1.25% |
| Erythritol | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Erythritol are then added to the resulting mixture and mixed under high shear to completely disperse the ingredients. The resulting filled polymer melt is cooled and ground to produce a powdered material with a particle size of less than 420 microns. The erythritol encapsulation matrix is stored in air tight containers with low humidity below 35° C.

### Example 4

### Encapsulation of Adipic acid—Polyvinyl acetate matrix

Composition:

| Ingredient | Weight percent |
|---|---|
| Polyvinyl Acetate | 60.00% |
| Hydrogenated Oil | 3.75% |
| Glycerol Monostearate | 1.25% |
| Adipic acid | 35.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Adipic acid is then added to the resulting mixture and mixed under high shear to completely disperse the ingredients. The resulting filled polymer melt is cooled and ground to produce a powdered material with a particle size of less than 420 microns. The encapsulated adipic acid matrix is stored in air tight containers with low humidity below 35° C.

### Example 5

### Encapsulation of Citric Acid—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Citric Acid | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Citric acid is then added to the resulting mixture and mixed under high shear to completely disperse the ingredients. The resulting filled polymer melt is cooled and ground to produce a powdered material with a particle size of less than 420 microns. The encapsulated citric acid matrix is stored in air tight containers with low humidity below 35° C.

### Example 6

### Encapsulation of Malic acid—Polyvinyl acetate

Composition:

| Ingredient | Weight percent |
|---|---|
| Polyvinyl Acetate | 55.00% |
| Hydrogenated Oil | 3.75% |
| Glycerol Monostearate | 1.25% |
| Malic acid | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Malic acid are then added to the resulting mixture and mixed under high shear to completely disperse the ingredients. The resulting filled polymer melt is cooled and ground to produce a powdered material with a particle size of less than 420 microns. The malic acid encapsulation matrix is stored in air tight containers with low humidity below 35° C.

### Example 7

### Encapsulation of Spray Dried Peppermint Flavor—Polyvinyl acetate

Composition:

| Ingredient | Weight percent |
|---|---|
| Polyvinyl Acetate | 75.00% |
| Hydrogenated Oil | 3.75% |
| Glycerol Monostearate | 1.25% |
| Spray dried peppermint flavor | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Spray dried peppermint flavor is then added to the resulting mixture and mixed under high shear to completely disperse the ingredients. The resulting filled polymer melt is cooled and ground to produce a powdered material with a particle size of less than 420 microns. The encapsulated peppermint flavor in Polyvinyl acetate matrix is stored in air tight containers with low humidity below 35° C.

### Example 8

### Encapsulation of Spray Dried Strawberry Flavor—Polyvinyl acetate

Composition:

| Ingredient | Weight percent |
|---|---|
| Polyvinyl Acetate | 55.00% |
| Hydrogenated Oil | 3.75% |
| Glycerol Monostearate | 1.25% |
| Spray dried strawberry flavor | 40.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Spray dried strawberry flavor is then added to the resulting mixture and mixed under high shear to completely disperse the ingredients. The resulting filled polymer melt is cooled and ground to produce a powdered material with a particle size of less than 420 microns. The encapsulated strawberry flavor is stored in air tight containers with low humidity below 35° C.

### Example 9

### Encapsulation of Monosodium Glutamate

Composition:

| Ingredient | Weight percent |
|---|---|
| Polyvinyl Acetate | 55.00% |
| Hydrogenated Oil | 3.75% |
| Glycerol Monostearate | 1.25% |
| Monosodium glutamate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Monosodium glutamate is then added to the resulting mixture and mixed under high shear to completely disperse the ingredients. The resulting filled polymer melt is cooled and ground to produce a powdered material with a particle size of less than 420 microns. The encapsulation matrix is stored in air tight containers with low humidity below 35° C.

### Example 10

### Encapsulation of Salt—Polyvinyl acetate matrix

Composition:

| Ingredient | Weight percent |
|---|---|
| Polyvinyl Acetate | 60.00% |
| Hydrogenated Oil | 3.75% |
| Glycerol Monostearate | 1.25% |
| Sodium chloride | 35.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sodium chloride is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 11

### Encapsulation of Sodium acid sulfate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium acid sulfate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sodium acid sulfate is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 13

### Encapsulation of WS-3 in Polyvinyl acetate

Composition:

| Ingredient | Weight percent |
|---|---|
| Polyvinyl Acetate | 65.00% |
| Hydrogenated Oil | 3.75% |
| Glycerol Monostearate | 1.25% |
| Cooling sensate WS-3 | 30.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. WS-3 is then added to the resulting mixture and mixed under high shear to completely disperse the ingredients. The resulting encapsulation is cooled and ground to produce a powdered material with a particle size of less than 420 microns. The malic acid encapsulation matrix is stored in air tight containers with low humidity below 35° C.

### Example 13

### Encapsulation of WS-23—Polyvinyl acetate matrix

Composition:

| Ingredient | Weight percent |
|---|---|
| Polyvinyl Acetate | 65.00% |
| Hydrogenated Oil | 3.75% |
| Glycerol Monostearate | 1.25% |
| Cooling sensate WS-23 | 30.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. WS-23 is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 14

### Encapsulation of Menthol—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Menthol | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Menthol crystals is then added to the resulting mixture and mixed under high shear to completely disperse the ingredients. The resulting polymer melt is cooled and ground to produce a powdered material with a particle size of less than 420 microns. The encapsulated menthol matrix is stored in air tight containers with low humidity below 35° C.

### Example 15

### Encapsulation of Caffeine—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Caffeine | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Caffeine is then added to the resulting mixture and mixed under high shear to completely disperse the ingredients. The resulting polymer melt is cooled and ground to produce a powdered material with a particle size of less than 420 microns. The encapsulated caffeine matrix is stored in air tight containers with low humidity below 35° C.

### Example 16

### Encapsulation of Ascorbic Acid—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Ascorbic Acid | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Ascorbic Acid is then added to the resulting mixture and mixed under high shear to completely disperse the ingredients. The resulting polymer melt is cooled and ground to produce a powdered material with a particle size of less than 420 microns. The encapsulated Ascorbic Acid matrix is stored in air tight containers with low humidity below 35° C.

### Example 17

### Encapsulation of Calcium Lactate—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Calcium Lactate | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Calcium Lactate is then added to the resulting mixture and mixed under high shear to completely disperse the ingredients. The resulting polymer melt is cooled and ground to produce a powdered material with a particle size of less than 420 microns. The encapsulated Calcium Lactate matrix is stored in air tight containers with low humidity below 35° C.

### Example 18

### Encapsulation of Zinc Citrate—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Zinc Citrate | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Zinc Citrate is then added to the resulting mixture and mixed under high shear to completely disperse the ingredients. The resulting polymer melt is cooled and ground to produce a powdered material with a particle size of less than 420 microns. The encapsulated Zinc Citrate matrix is stored in air tight containers with low humidity below 35° C.

### Example 19

### Encapsulation of Niacin—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Niacin | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Niacin is then added to the resulting mixture and mixed under high shear to completely disperse the ingredients. The resulting polymer melt is cooled and ground to produce a powdered material with a particle size of less than 420 microns. The encapsulated Niacin matrix is stored in air tight containers with low humidity below 35° C.

### Example 20

### Encapsulation of Pyridoxine—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Pyridoxine | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Pyridoxine is then added to the resulting mixture and mixed under high shear to completely disperse the ingredients. The resulting polymer melt is cooled and ground to produce a powdered material with a particle size of less than 420 microns. The encapsulated Pyridoxine matrix is stored in air tight containers with low humidity below 35° C.

### Example 21

### Encapsulation of Thiamine—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Thiamine | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Thiamine is then added to the resulting mixture and mixed under high shear to completely disperse the ingredients. The resulting polymer melt is cooled and ground to produce a powdered material with a particle size of less than 420 microns. The encapsulated Thiamine matrix is stored in air tight containers with low humidity below 35° C.

### Example 22

### Encapsulation of Riboflavin—Polyvinyl acetate matrix

Composition:

| Ingredient | Weight percent |
|---|---|
| Polyvinyl Acetate | 75.00% |
| Hydrogenated Oil | 3.75% |
| Glycerol Monostearate | 1.25% |
| Riboflavin | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Riboflavin is then added to the resulting mixture and mixed under high shear to completely disperse the ingredients. The resulting polymer melt is cooled and ground to produce a powdered material with a particle size of less than 420 microns. The encapsulated Riboflavin matrix is stored in air tight containers with low humidity below 35° C.

### Example 23

### Encapsulation of Sucralose—Polyvinyl acetate matrix (Sucralose 20%)

Composition:

| Ingredient | Weight percent |
|---|---|
| Polyvinyl Acetate | 77.00% |
| Hydrogenated Oil | 3.00% |
| Sucralose | 20.00% |
| Total | 100.00% |

Procedure: Polyvinyl acetate is melted at a temperature of about 85° C. in a high shear mixer such as extruder (single or twin screw) or sigma or Banbury mixer. The hydrogenated oil is added to the molten polyvinyl acetate. Sucralose is then added to the resulting mixture and mixed under high shear to completely disperse the ingredients. The resulting filled polymer melt is cooled and ground to produce a powdered material with a particle size of less than 590 microns. The encapsulated sucralose matrix is stored in air tight containers with low humidity below 35° C.

### Example 24

### Multiple Encapsulation of Sucralose/Polyvinyl acetate matrix (from example 23)

Composition:

| Ingredient | Grams |
|---|---|
| Center Cores | |
| Sucralose/Polymer Matrix (from Example 23) | 700.0 |
| Coating Solution | |
| Purified Water | 1168.0 |
| Gum Arabic | 293.0 |
| Total Coating solution | 1461.0 |

Procedure: Wurster process is used to encapsulate Sucralose/Polymer Matrix. Coating solution using the above mentioned recipe is prepared by stirring water and gum at 35° C. for 2 hrs. 700 gms of Sucralose//Polymer Matrix are suspended in a fluidizing air stream which provide generally cyclic flow in front of a spray nozzle. The spray nozzle sprays an atomized flow of 1461 gms of the coating solution for 115 minutes. The coated particles are then dried in the fluidized chamber for 50 minutes and stored below 35° C. under dry conditions.

### Example 25

### High Tensile Strength Encapsulation of Aspartame—Polyvinyl acetate matrix (Aspartame 30%) Particle Size Less than 420 Microns

Composition:

| Ingredient | Weight percent |
|---|---|
| Polyvinyl Acetate | 65.00% |
| Hydrogenated Oil | 3.75% |
| Glycerol Monostearate | 1.25% |
| Aspartame | 30.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame is then added to the resulting mixture and mixed under high shear to completely disperse the ingredients. The resulting high tensile strength/low fat content encapsulation is cooled and ground to produce a powdered material with a particle size of less than 420 microns.

### Example 25 B

### Low Tensile Strength Encapsulation of Aspartame—Polyvinyl acetate matrix (Aspartame 30%)

Composition:

| Ingredient | Weight percent |
|---|---|
| Polyvinyl Acetate | 50.00% |
| Hydrogenated Oil | 10.00% |
| Glycerol Monostearate | 10.00% |
| Aspartame | 30.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame is then added to the resulting mixture and mixed under high shear to completely disperse the ingredients. The resulting low Tensile Strength encapsulation is cooled and ground to produce a powdered material with a particle size of less than 420 microns.

### Example 25 C

### High Tensile Strength Encapsulation of Aspartame—Polyvinyl acetate matrix (Aspartame 30%) Particle Size Less than 177 microns

Composition:

| Ingredient | Weight percent |
|---|---|
| Polyvinyl Acetate | 65.00% |
| Hydrogenated Oil | 3.75% |
| Glycerol Monostearate | 1.25% |
| Aspartame | 30.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame is then added to the resulting mixture and mixed under high shear to completely disperse the ingredients. The resulting high tensile strength/low fat content encapsulation is cooled and ground to produce a powdered material with a particle size of less than 177 microns.

### Example 26

### Encapsulation of AceK—Polyvinyl acetate matrix (AceK 30%)

Composition:

| Ingredient | Weight percent |
|---|---|
| Polyvinyl Acetate | 65.00% |
| Hydrogenated Oil | 3.75% |
| Glycerol Monostearate | 1.25% |
| AceK | 30.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. AceK is then added to the resulting mixture and mixed under high shear to completely disperse the ingredients. The resulting filled polymer melt is cooled and ground to produce a powdered material with a particle size of less than 420 microns. The encapsulated AceK matrix is stored in air tight containers with low humidity below 35° C.

### Example 27

### Encapsulation of Neotame—Polyvinyl acetate matrix (Neotame 10%)

Composition:

| Ingredient | Weight percent |
|---|---|
| Polyvinyl Acetate | 75.00% |
| Hydrogenated Oil | 10.00% |
| Glycerol Monostearate | 5.00% |
| Neotame | 10.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Neotame is then added to the resulting mixture and mixed under high shear to completely disperse the ingredients. The resulting filled polymer melt is cooled and ground to produce a powdered material with a particle size of less than 420 microns. The encapsulated Neotame polymer encapsulation particles are stored in air tight containers with low humidity below 35° C.

### Example 28

### Encapsulation of Pectin in Polyvinyl acetate matrix (Pectin 30%)

Composition:

| Ingredient | Weight percent |
|---|---|
| Polyvinyl Acetate | 65.00% |
| Hydrogenated Oil | 3.75% |
| Glycerol Monostearate | 1.25% |
| Pectin | 30.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Pectin is then added to the resulting mixture and mixed under high shear to completely disperse the ingredients. The resulting filled polymer melt is cooled and ground to produce a powdered material with a particle size of less than 420 microns. The encapsulated pectin polymer encapsulation particles are stored in air tight containers with low humidity below 35° C.

### Example 50

### Chewing Gum Composition Containing Encapsulated Glycyrrhizin

| Ingredient | Weight percent |
|---|---|
| Gum Base | 39.00 |
| Sorbitol | 45.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Glycyrrhizin (from Example 1) | 1.10 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 51

### Chewing Gum Composition Containing Encapsulated Xylitol

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Xylitol (from Example 2) | 6.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 52

### Chewing Gum Composition Containing Encapsulated Erythritol

| Ingredient | Weight percent |
|---|---|
| Gum Base | 39.00 |
| Sorbitol | 40.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Erythritol (from Example 3) | 6.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 53

### Chewing Gum Composition Containing Encapsulated Adipic Acid—Polyvinyl acetate matrix

| Ingredient | Weight percent |
|---|---|
| Gum Base | 39.00 |
| Sorbitol | 42.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Adipic Acid (from Example 4) | 4.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 54

### Chewing Gum Composition Containing Encapsulated Citric Acid—Polyvinyl acetate matrix

| Ingredient | Weight percent |
|---|---|
| Gum Base | 39.00 |
| Sorbitol | 42.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Citric Acid (from Example 5) | 4.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 55

### Chewing Gum Composition Containing Encapsulated Malic acid—Polyvinyl acetate

| Ingredient | Weight percent |
|---|---|
| Gum Base | 39.00 |
| Sorbitol | 42.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Malic Acid (from Example 6) | 4.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 56

### Chewing Gum Composition Containing Encapsulated Spray Dried Peppermint Flavor

| Ingredient | Weight percent |
|---|---|
| Gum Base | 39.00 |
| Sorbitol | 40.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Spray Dried Peppermint Flavor (from | 6.00 |
| Example 7) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 57

### Chewing Gum Composition Containing Encapsulated Spray Dried Strawberry Flavor

| Ingredient | Weight percent |
|---|---|
| Gum Base | 39.00 |
| Sorbitol | 40.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Spray dried strawberry flavor | 6.00 |
| (from Example 8) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 58

### Chewing Gum Composition Containing Encapsulated Monosodium Glutamate

| Ingredient | Weight percent |
|---|---|
| Gum Base | 39.00 |
| Sorbitol | 42.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Monosodium Glutamate (from Example 9) | 4.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 59

### Chewing Gum Composition Containing Encapsulated Salt

| Ingredient | Weight percent |
|---|---|
| Gum Base | 39.00 |
| Sorbitol | 42.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Salt (from Example 10) | 4.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 60

### Chewing Gum Composition Containing Encapsulated Sodium acid sulfate

| Ingredient | Weight percent |
|---|---|
| Gum Base | 39.00 |
| Sorbitol | 41.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodium acid sulfate | 5.00 |
| (from Example 11) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 61

### Chewing Gum Composition Containing Encapsulated WS-3

| Ingredient | Weight percent |
|---|---|
| Gum Base | 39.00 |
| Sorbitol | 44.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated WS-3 (from Example 12) | 2.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 62

### Chewing Gum Composition Containing Encapsulated WS-23

| Ingredient | Weight percent |
|---|---|
| Gum Base | 39.00 |
| Sorbitol | 44.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated WS-23 (from Example 13) | 2.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 63

### Chewing Gum Composition Containing Encapsulated Menthol

| Ingredient | Weight percent |
|---|---|
| Gum Base | 39.00 |
| Sorbitol | 43.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Menthol (from Example 14) | 3.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 64

### Chewing Gum Composition Containing Encapsulated Caffeine

| Ingredient | Weight percent |
|---|---|
| Gum Base | 39.00 |
| Sorbitol | 43.78 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Caffeine (from Example 15) | 1.50 |
| Encapsulated sucralose (from example 23) | 0.90 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Using encapsulated sucralose with encapsulated caffeine will result in controlled release of sucralose and caffeine. This will result in masking of bitterness from caffeine release.

### Example 65

### Chewing Gum Composition Containing Encapsulated Ascorbic Acid

| Ingredient | Weight percent |
|---|---|
| Gum Base | 39.00 |
| Sorbitol | 43.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Ascorbic Acid | 3.00 |
| (from Example 16) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 66

### Chewing Gum Composition Containing Encapsulated Calcium Lactate

| Ingredient | Weight percent |
|---|---|
| Gum Base | 39.00 |
| Sorbitol | 41.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Calcium Lactate | 5.00 |
| (from Example 17) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and Packaged.

### Example 67

### Chewining Gum Composition Containing Encapsulated Zinc Citrate

| Ingredient | Weight percent |
|---|---|
| Gum Base | 39.00 |
| Sorbitol | 42.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Zinc Citrate | 4.00 |
| (from Example 18) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 68

### Chewing Gum Composition Containing Encapsulated Niacin

| Ingredient | Weight percent |
|---|---|
| Gum Base | 39.00 |
| Sorbitol | 43.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Niacin (from Example 19) | 3.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 69

### Chewing Gum Composition Containing Encapsulated Pyridoxine

| Ingredient | Weight percent |
|---|---|
| Gum Base | 39.00 |
| Sorbitol | 45.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Pyridoxine (from Example 20) | 1.10 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 70

### Chewing Gum Composition Containing Encapsulated Thiamine

| Ingredient | Weight percent |
|---|---|
| Gum Base | 39.00 |
| Sorbitol | 45.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Thiamine (from Example 21) | 1.10 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 71

### Chewing Gum Composition Containing Encapsulated Riboflavin

| Ingredient | Weight percent |
|---|---|
| Gum Base | 39.00 |
| Sorbitol | 45.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Riboflavin (from Example 22) | 1.10 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.
Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Chew out-release studies of this gum shows controlled/slower release as compared to gum in example 72 as shown in FIG. 1.

### Example 75 A

### Chewing Gum Composition Containing High Tensile Strength Encapsulated Aspartame (Particle Size Less than 420 Microns) and AceK encapsulated individually

| Ingredient | Weight percent |
|---|---|
| Gum Base | 39.00 |
| Sorbitol | 44.30 |
| Mannitol | 9.00 |
| Flavor | 3.67 |
| Glycerin | 1.50 |
| Lecithin | 0.20 |
| Encapsulated aspartame | 1.63 |
| from example 25 A (30% active) | |
| Encapsulated AceK from | 0.70 |
| example 26 (30% active) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Chew out studies on this gums shows slower aspartame release compared to example 75B (with low strength encapsulated aspartame) and 76 (with aspartame).

### Example 75 B

### Chewing Gum Composition Containing Low Tensile Strength Encapsulated Aspartame and AceK, encapsulated individually

| Ingredient | Weight percent |
|---|---|
| Gum Base | 39.00 |
| Sorbitol | 44.30 |
| Mannitol | 9.00 |
| Flavor | 3.67 |
| Glycerin | 1.50 |
| Lecithin | 0.20 |
| Encapsulated aspartame | 1.63 |
| from example 25 B (30% active) | |
| Encapsulated AceK from | 0.70 |
| example 26 (30% active) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Chew out studies on this gums shows faster aspartame release compared to gum in example 75 A (with high strength encapsulated aspartame) but slower than gum made in example 76 (with aspartame).

### Example 75 C

### Chewing Gum Composition Containing High Tensile Strength Encapsulated Aspartame (Particle Size Less than 177 Microns) and AceK encapsulated individually

| Ingredient | Weight percent |
|---|---|
| Gum Base | 39.00 |
| Sorbitol | 44.30 |
| Mannitol | 9.00 |
| Flavor | 3.67 |
| Glycerin | 1.50 |
| Lecithin | 0.20 |
| Encapsulated aspartame | 1.63 |
| from example 25 C (30% active) | |
| Encapsulated AceK from | 0.70 |
| example 26 (30% active) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Chew out studies on this gums shows faster aspartame release compared to example 75A with larger encapsulation particle size.

### Example 76

### Chewing Gum Composition Containing Aspartame and AceK

| Ingredient | Weight percent |
|---|---|
| Gum Base | 39.00 |
| Sorbitol | 45.93 |
| Mannitol | 9.00 |
| Flavor | 3.67 |
| Glycerin | 1.50 |
| Lecithin | 0.20 |
| Aspartame | 0.49 |
| AceK | 0.21 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 77

### Chewing Gum Composition Containing Aspartame, AceK and Encapsulated Neotame

| Ingredient | Weight percent |
|---|---|
| Gum Base | 39.00 |
| Sorbitol | 45.35 |
| Mannitol | 9.00 |
| Flavor | 3.67 |
| Glycerin | 1.50 |
| Lecithin | 0.20 |
| Aspartame | 0.60 |
| Acek | 0.38 |
| Encapsulated Neotame | 0.30 |
| from example 27 | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 78

### Chewing Gum Composition Containing Encapsulated Pectin

| Ingredient | Weight percent |
|---|---|
| Gum Base | 39.00 |
| Sorbitol | 42.55 |
| Mannitol | 9.00 |
| Flavor | 3.67 |
| Glycerin | 1.50 |
| Lecithin | 0.20 |
| Aspartame | 0.60 |
| Acek | 0.38 |
| Encapsulated Pectin | 3.10 |
| from example 28. | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Ingredient Examples of Multiple Ingredients in a Delivery System

### Example 101

### Encapsulation of Aspartame, Ace-K, and Sucralose

Composition :

| Ingredient | Weight percent |
|---|---|
| Polyvinyl Acetate | 55.00% |
| Hydrogenated Oil | 3.75% |
| Glycerol Monostearate | 1.25% |
| Aspartame | 20.00% |
| AceK | 10.00% |
| Sucralose | 10.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Aspartame, Ace-K, and Sucralose are then added to the resulting mixture and mixed under high shear to completely disperse the ingredients. The resulting filled polymer melt is cooled and ground to produce a powdered material with a particle size of less than 420 microns. The encapsulated sweeteners are stored in air tight containers with low humidity below 35° C.

### Example 102

### Encapsulation of Aspartame, Ace-K, and Glycyrrhizin

Composition:

| Ingredient | Weight percent |
|---|---|
| Polyvinyl Acetate | 55.00% |
| Hydrogenated Oil | 3.75% |
| Glycerol Monostearate | 1.25% |
| Aspartame | 20.00% |
| Ace-K | 10.00% |
| Glycyrrhizin | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame, Ace-K, and Glycyrrhizin are then added to the resulting mixture and mixed under high shear to completely disperse the ingredients. The resulting filled polymer melt is cooled and ground to produce a powdered material with a particle size of less than 420 microns. The encapsulated sweeteners are stored in air tight containers with low humidity below 35° C.

### Example 103

### Encapsulation of Aspartame, Ace-K, and Menthol

| Ingredient | Weight percent |
|---|---|
| Polyvinyl Acetate | 55.00% |
| Hydrogenated Oil | 3.75% |
| Glycerol Monostearate | 1.25% |
| Aspartame | 20.00% |
| Ace-K | 10.00% |
| Menthol | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame, Ace-K, and Menthol are then added to the resulting mixture and mixed under high shear to completely disperse the ingredients. The resulting filled polymer melt is cooled and ground to produce a powdered material with a particle size of less than 420 microns. The encapsulated sweeteners are stored in air tight containers with low humidity below 35° C.

### Example 104

### Encapsulation of Aspartame, Ace-K, and Adipic Acid

Composition:

| Ingredient | Weight percent |
|---|---|
| Polyvinyl Acetate | 55.00% |
| Hydrogenated Oil | 3.75% |
| Glycerol Monostearate | 1.25% |
| Aspartame | 10.00% |
| Ace-K | 5.00% |
| Adipic acid | 25.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame, Ace-K, and Adipic Acid are then added to the resulting mixture and mixed under high shear to completely disperse the ingredients. The resulting filled polymer melt is cooled and ground to produce a powdered material with a particle size of less than 420 microns. The encapsulated sweeteners are stored in air tight containers with low humidity below 35° C.

### Example 105

### Encapsulation of Adipic, Citric, and Malic Acid

Composition:

| Ingredient | Weight percent |
|---|---|
| Polyvinyl Acetate | 55.00% |
| Hydrogenated Oil | 3.75% |
| Glycerol Monostearate | 1.25% |
| Adipic Acid | 10.00% |
| Citric Acid | 20.00% |
| Malic Acid | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Adipic, Citric, and Malic Acid are then added to the resulting mixture and mixed under high shear to completely disperse the ingredients. The resulting filled polymer melt is cooled and ground to produce a powdered material with a particle size of less than 420 microns. The encapsulated acids are stored in air tight containers with low humidity below 35° C.

### Example 106

### Encapsulation of Sucralose, and Citric Acid

Composition:

| Ingredient | Weight percent |
|---|---|
| Polyvinyl Acetate | 55.00% |
| Hydrogenated Oil | 3.75% |
| Glycerol Monostearate | 1.25% |
| Sucralose | 10.00% |
| Citric Acid | 30.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sucralose and Citric Acid are then added to the resulting mixture and mixed under high shear to completely disperse the ingredients. The resulting filled polymer melt is cooled and ground to produce a powdered material with a particle size of less than 420 microns. The encapsulation is stored in air tight containers with low humidity below 35° C.

### Example 107

### Encapsulation of Sucralose and Adipic Acid

Composition:

| Ingredient | Weight percent |
|---|---|
| Polyvinyl Acetate | 55.00% |
| Hydrogenated Oil | 3.75% |
| Glycerol Monostearate | 1.25% |
| Sucralose | 10.00% |
| Adipic Acid | 30.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Sucralose and Adipic Acid are then added to the resulting mixture and mixed under high shear to completely disperse the ingredients. The resulting filled polymer melt is cooled and ground to produce a powdered material with a particle size of less than 420 microns. The encapsulation is stored in air tight containers with low humidity below 35° C.

### Example 108

### Encapsulation of Aspartame and Salt

Composition:

| Ingredient | Weight percent |
|---|---|
| Polyvinyl Acetate | 55.00% |
| Hydrogenated Oil | 3.75% |
| Glycerol Monostearate | 1.25% |
| Aspartame | 20.00% |
| Salt | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Aspartame and Salt are then added to the resulting mixture and mixed under high shear to completely disperse the ingredients. The resulting filled polymer melt is cooled and ground to produce a powdered material with a particle size of less than 420 microns. The encapsulation is stored in air tight containers with low humidity below 35° C.

### Example 109

### Encapsulation of Aspartame with WS-3

Composition:

| Ingredient | Weight percent |
|---|---|
| Polyvinyl Acetate | 65.00% |
| Hydrogenated Oil | 3.75% |
| Glycerol Monostearate | 1.25% |
| Aspartame | 20.00% |
| WS-3 | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame and WS-3 are then added to the resulting mixture and mixed under high shear to completely disperse the ingredients. The resulting filled polymer melt is cooled and ground to produce a powdered material with a particle size of less than 420 microns. The encapsulation is stored in air tight containers with low humidity below 35° C.

### Example 110

### Encapsulation of Sucralose with WS-23

Composition:

| Ingredient | Weight percent |
|---|---|
| Polyvinyl Acetate | 75.00% |
| Hydrogenated Oil | 3.75% |
| Glycerol Monostearate | 1.25% |
| Sucralose | 10.00% |
| WS-23 | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sucralose and WS-23 are then added to the resulting mixture and mixed under high shear to completely disperse the ingredients. The resulting filled polymer melt is cooled and ground to produce a powdered material with a particle size of less than 420 microns. The encapsulation is stored in air tight containers with low humidity below 35° C.

### Example 111

### Encapsulation of Sucralose and Menthol

Composition:

| Ingredient | Weight percent |
|---|---|
| Polyvinyl Acetate | 70.00% |
| Hydrogenated Oil | 3.75% |
| Glycerol Monostearate | 1.25% |
| Sucralose | 10.00% |
| Menthol | 15.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sucralose and Menthol are then added to the resulting mixture and mixed under high shear to completely disperse the ingredients. The resulting filled polymer melt is cooled and ground to produce a powdered material with a particle size of less than 420 microns. The encapsulation is stored in air tight containers with low humidity below 35° C.

### Example 112

### Encapsulation of Aspartame and Neotame

Composition:

| Ingredient | Weight percent |
|---|---|
| Polyvinyl Acetate | 60.00% |
| Hydrogenated Oil | 3.75% |
| Glycerol Monostearate | 1.25% |
| Aspartame | 30.00% |
| Neotame | 5.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Aspartame and Neotame are then added to the resulting mixture and mixed under high shear to completely disperse the ingredients. The resulting encapsulation is cooled and ground to produce a powdered material with a particle size of less than 420 microns. The encapsulation matrix is stored in air tight containers with low humidity below 35° C.

### Example 113

### Encapsulation of Aspartame and Adenosine monophosphate (Bitterness Inhibitor)

Composition:

| Ingredient | Weight percent |
|---|---|
| Polyvinyl Acetate | 65.00% |
| Hydrogenated Oil | 3.75% |
| Glycerol Monostearate | 1.25% |
| Aspartame | 20.00% |
| Adenosine monophosphate (AMP) | 10.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Aspartame and AMP are then added to the resulting mixture and mixed under high shear to completely disperse the ingredients. The resulting filled polymer melt is cooled and ground to produce a powdered material with a particle size of less than 420 microns. The encapsulation is stored in air tight containers with low humidity below 35° C.

### Example 114

### Encapsulation of Aspartame and Caffeine

Composition:

| Ingredient | Weight percent |
|---|---|
| Polyvinyl Acetate | 60.00% |
| Hydrogenated Oil | 3.75% |
| Glycerol Monostearate | 1.25% |
| Aspartame | 20.00% |
| Caffeine | 15.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Aspartame and Caffeine are then added to the resulting mixture and mixed under high shear to completely disperse the ingredients. The resulting polymer melt is cooled and ground to produce a powdered material with a particle size of less than 420 microns. The encapsulation is stored in air tight containers with low humidity below 35° C.

### Example 115

### Encapsulation of Sucralose and Calcium Lactate

Composition:

| Ingredient | Weight percent |
|---|---|
| Polyvinyl Acetate | 55.00% |
| Hydrogenated Oil | 3.75% |
| Glycerol Monostearate | 1.25% |
| sucralose | 10.00% |
| Calcium Lactate | 30.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Aspartame and Calcium Lactate are then added to the resulting mixture and mixed under high shear to completely disperse the ingredients. The resulting polymer melt is cooled and ground to produce a powdered material with a particle size of less than 420 microns. The encapsulation is stored in air tight containers with low humidity below 35° C.

### Example 116

### Encapsulation of Sucralose and Vitamin C

Composition:

| Ingredient | Weight percent |
|---|---|
| Polyvinyl Acetate | 65.00% |
| Hydrogenated Oil | 3.75% |
| Glycerol Monostearate | 1.25% |
| Sucralose | 10.00% |
| Ascorbic Acid (Vitamin C) | 20.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Sucralose and Ascorbic Acid is then added to the resulting mixture and mixed under high shear to completely disperse the ingredients. The resulting polymer melt is cooled and ground to produce a powdered material with a particle size of less than 420 microns. The encapsulation is stored in air tight containers with low humidity below 35° C.

### Example 117

### Encapsulation of Aspartame and Niacin

Composition:

| Ingredient | Weight percent |
|---|---|
| Polyvinyl Acetate | 65.00% |
| Hydrogenated Oil | 3.75% |
| Glycerol Monostearate | 1.25% |
| Aspartame | 15.00% |
| Niacin | 15.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Aspartame and Niacin are then added to the resulting mixture and mixed under high shear to completely disperse the ingredients. The resulting polymer melt is cooled and ground to produce a powdered material with a particle size of less than 420 microns. The encapsulation is stored in air tight containers with low humidity below 35° C.

### Example 118

### Encapsulation of Sucralose and Folic Acid

Composition:

| Ingredient | Weight percent |
|---|---|
| Polyvinyl Acetate | 75.00% |
| Hydrogenated Oil | 3.75% |
| Glycerol Monostearate | 1.25% |
| Sucralose | 10.00% |
| Folic Acid | 10.00% |
| Total | 100.00% |

Procedure: Polyvinyl acetate is melted at a temperature of about 90° C. in a high shear mixer such as extruder (single or twin screw) or sigma or Banbury mixer. The hydrogenated oil and Glycerol monostearate are then added to the molten polyvinyl acetate. Sucralose and Folic Acid are then added to the resulting mixture and mixed under high shear to completely disperse the ingredients. The resulting polymer melt is cooled and ground to produce a powdered material with a particle size of less than 420 microns. The encapsulation is stored in air tight containers with low humidity below 35° C.

### Example 119

### Encapsulation of Mixed Aspartame and AceK—Polyvinyl acetate matrix (Actives=30%)

Composition:

| Ingredient | Weight percent |
|---|---|
| Polyvinyl Acetate | 65.00% |
| Hydrogenated Oil | 3.75% |
| Glycerol Monostearate | 1.25% |
| Aspartame | 21.00% |
| AceK | 9.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Aspartame and AceK (60/40) are then added to the resulting mixture and mixed under high shear to completely disperse the ingredients. The resulting filled polymer melt is cooled and ground to produce a powdered material with a particle size of less than 420 microns. The mixed Aspartame and AceK encapsulation matrix is stored in air tight containers with low humidity below 35° C.

### Example 120

### Encapsulation of Mixed WS-3 and WS-23—Polyvinyl acetate matrix

Composition:

| Ingredient | Weight percent |
|---|---|
| Polyvinyl Acetate | 65.00% |
| Hydrogenated Oil | 3.75% |
| Glycerol Monostearate | 1.25% |
| Cooling sensate WS-3 | 15.00% |
| Cooling sensate WS-23 | 15.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. WS-3 and WS-23 are then added to the resulting mixture and mixed under high shear to completely disperse the ingredients. The resulting filled polymer melt is cooled and ground to produce a powdered material with a particle size of less than 420 microns. The mixed WS-3 and WS-23 encapsulation matrix is stored in air tight containers with low humidity below 35° C.

### Example 121

### Encapsulation of Mixed Aspartame and Calciumcarbonate—Polyvinyl acetate matrix

Composition:

| Ingredient | Weight percent |
|---|---|
| Polyvinyl Acetate | 60.00% |
| Hydrogenated Oil | 3.75% |
| Glycerol Monostearate | 1.25% |
| Aspartame | 20.00% |
| Calciumcarbonate | 15.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Aspartame and calcium carbonate are then added to the resulting mixture and mixed under high shear to completely disperse the ingredients. The resulting filled polymer melt is cooled and ground to produce a powdered material with a particle size of less than 420 microns. The mixed aspartame and calcium carbonate encapsulation matrix is stored in air tight containers with low humidity below 35° C.

### Example 122

### Encapsulation of Mixed Aspartame and Talc—Polyvinyl acetate matrix

Composition:

| Ingredient | Weight percent |
|---|---|
| Polyvinyl Acetate | 60.00% |
| Hydrogenated Oil | 3.75% |
| Glycerol Monostearate | 1.25% |
| Aspartame | 20.00% |
| Talc | 15.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Aspartame and talc are then added to the resulting mixture and mixed under high shear to completely disperse the ingredients. The resulting filled polymer melt is cooled and ground to produce a powdered material with a particle size of less than 420 microns. The mixed aspartame and talc encapsulation matrix is stored in air tight containers with low humidity below 35° C.

### Example 151

### Chewing Gum Composition Containing Encapsulated Aspartame, Ace-K, and Sucralose

| Ingredient | Weight percent |
|---|---|
| Gum Base | 39.00 |
| Sorbitol | 44.18 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame, Ace-K, and Sucralose | 2.00 |
| (from Example 101) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 152

### Chewing Gum Composition Containing Encapsulated Aspartame, Ace-K, and Glycyrrhizin

| Ingredient | Weight percent |
|---|---|
| Gum Base | 39.00 |
| Sorbitol | 45.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame, Ace-K, and Glycyrrhizin (from | 1.10 |
| Example 102) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 153

### Chewing Gum Composition Containing Encapsulated Aspartame, Ace-K, and Methol

| Ingredient | Weight percent |
|---|---|
| Gum Base | 39.00 |
| Sorbitol | 43.68 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame, Ace-K, and Menthol | 2.50 |
| (from Example 103) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 154

### Chewing Gum Composition Containing Encapsulated Aspartame, Ace-K, and Adipic Acid

| Ingredient | Weight percent |
|---|---|
| Gum Base | 39.00 |
| Sorbitol | 42.98 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame, Ace-K, and Adipic Acid | 3.20 |
| (from Example 104) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 155

### Chewing Gum Composition Containing Encapsulated Adipic, Citric, and Malic acid

| Ingredient | Weight percent |
|---|---|
| Gum Base | 39.00 |
| Sorbitol | 41.98 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Adipic, Citric, and Malic Acid | 4.20 |
| (from Example 105) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 156

### Chewing Gum Composition Containing Encapsulated Sucralose and Citric Acid

| Ingredient | Weight percent |
|---|---|
| Gum Base | 39.00 |
| Sorbitol | 44.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sucralose and Citric Acid | 2.10 |
| (from Example 106) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 157

### Chewing Gum Composition Containing Encapsulated Sucralose and Adipic Acid

| Ingredient | Weight percent |
|---|---|
| Gum Base | 39.00 |
| Sorbitol | 44.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sucralose and Adipic Acid | 2.10 |
| (from Example 107) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 158

### Chewing Gum Composition Containing Encapsulated Aspartame and Salt

| Ingredient | Weight percent |
|---|---|
| Gum Base | 39.00 |
| Sorbitol | 42.98 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame and Salt | 3.20 |
| (from Example 108) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 159

### Chewing Gum Composition Containing Encapsulated Aspartame and WS-3

| Ingredient | Weight percent |
|---|---|
| Gum Base | 39.00 |
| Sorbitol | 43.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame with WS-3 | 3.10 |
| (from Example 109) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 160

### Chewing Gum Composition Containing Encapsulated Sucralose with WS-23

| Ingredient | Weight percent |
|---|---|
| Gum Base | 39.00 |
| Sorbitol | 44.38 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sucralose with WS-23 | 1.80 |
| (from Example 110) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 161

### Chewing Gum Composition Containing Encapsulated Sucralose with Menthol

| Ingredient | Weight percent |
|---|---|
| Gum Base | 39.00 |
| Sorbitol | 44.08 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sucralose with Menthol | 2.10 |
| (from Example 111) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 162

### Chewing Gum Composition Containing Encapsulated Aspartame with Neotame

| Ingredient | Weight percent |
|---|---|
| Gum Base | 39.00 |
| Sorbitol | 42.28 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame with Neotame | 3.90 |
| (from Example 112) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 163

### Chewing Gum Composition Containing Encapsulated Aspartame with AMP

| Ingredient | Weight percent |
|---|---|
| Gum Base | 39.00 |
| Sorbitol | 41.58 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame with AMP | 4.60 |
| (from Example 113) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 164

### Chewing Gum Composition Containing Encapsulated Aspartame with Caffeine

| Ingredient | Weight percent |
|---|---|
| Gum Base | 39.00 |
| Sorbitol | 43.58 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame with Caffeine | 2.60 |
| (from Example 114) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 165

### Chewing Gum Composition Containing Encapsulated Aspartame with Calcium Lactate

| Ingredient | Weight percent |
|---|---|
| Gum Base | 39.00 |
| Sorbitol | 40.98 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame with Calcium | 5.20 |
| Lactate (from Example 115) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 166

### Chewing Gum Composition Containing Encapsulated Sucralose with Vitamin C

| Ingredient | Weight percent |
|---|---|
| Gum Base | 39.00 |
| Sorbitol | 42.28 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sucralose with Vitamin C | 3.90 |
| (from Example 116) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 167

### Chewing Gum Composition Containing Encapsulated Aspartame with Niacin

| Ingredient | Weight percent |
|---|---|
| Gum Base | 39.00 |
| Sorbitol | 43.28 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Aspartame with Niacin | 2.90 |
| (from Example 117) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 168

### Chewing Gum Composition Containing Encapsulated Sucralose with Folic Acid

| Ingredient | Weight percent |
|---|---|
| Gum Base | 39.00 |
| Sorbitol | 43.98 |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated sucralose with Folic Acid | 2.20 |
| (from Example 118) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 169

### Chewing Gum Composition Containing Encapsulated Aspartame and AceK (mixed) Encapsulated

| Ingredient | Weight percent |
|---|---|
| Gum Base | 39.00 |
| Sorbitol | 44.30 |
| Mannitol | 9.00 |
| Flavor | 3.67 |
| Glycerin | 1.50 |
| Lecithin | 0.20 |
| Encapsulated Aspartame + AceK from | 2.33 |
| example 119 (30% active) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 170

### Chewing Gum Composition Containing WS-3 and WS-23 Encapsulated in Single Polymer Matrix (from Example 120)

| Ingredient | Weight percent |
|---|---|
| Gum Base | 39.00 |
| Sorbitol | 44.30 |
| Mannitol | 9.00 |
| Flavor | 3.67 |
| Glycerin | 1.50 |
| Lecithin | 0.20 |
| Encapsulated WS-3 and WS-23 from | 2.33 |
| example 120 (30% active) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Ingredient Examples of Single Oral Care Ingredients in a Delivery System

### Example 300

### Encapsulation of Sodium tripolyphosphate (Sodiumtripolyphospate)—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodiumtripolyphosphate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sodiumtripolyphosphate is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 301

### Encapsulation of Sodium Fluoride (NaF)—Polyvinyl acetate matrix

Composition:

| Ingredient | Weight percent |
|---|---|
| Polyvinyl Acetate | 65.00% |
| Hydrogenated Oil | 3.75% |
| Glycerol Monostearate | 1.25% |
| Sodium Fluoride | 30.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. NaF is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 302

### Encapsulation of Calcium peroxide—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Calcium Peroxide | 40.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Calcium peroxide is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 303

### Encapsulation of Zinc Chloride—Polyvinyl acetate matrix

Composition:

| Ingredient | Weight percent |
|---|---|
| Polyvinyl Acetate | 65.00% |
| Hydrogenated Oil | 3.75% |
| Glycerol Monostearate | 1.25% |
| Zinc Chloride | 30.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. zinc chloride is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 304

### Encapsulation of Carbamide peroxide—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Carbamide Peroxide | 40.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Carbamide peroxide is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 305

### Encapsulation of Potassium Nitrate (KNO3)—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Potassium Nitrate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. KNO3 is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 306

### Encapsulation of Chlorhexidine—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Chlorhexidine | 40.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Chlorhexidine is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 307

### Encapsulation of Sodium stearate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium stearate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sodium stearate is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 308

### Encapsulation of Sodium Bicarbonate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium Bicarbonate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. NaHCO3 is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 309

### Encapsulation of Cetylpridinium chloride (CPC)—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Cetylpridinium chloride | 40.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. CPC is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 310

### Encapsulation of Calcium Casein Peptone-Calcium Phosphate CCP-CP (Recaldent)—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Recaldent | 40.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Recaldent is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 311

### Encapsulation of Sodium Ricinoleate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium Ricinoleate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sodium ricinoleate is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 312

### Encapsulation of Sodium hexametaphosphate (Sodiumhexamataphosphate)—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium Hexametaphosphate | 40.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sodiumhexamataphosphate is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 313

### Encapsulation of Urea—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Urea | 40.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Urea is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 314

### Chewing Gum Composition Containing Encapsulated Sodium tripolyphosphate (Sodiumtripolyphosphate)

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodiumtripolyphosphate | 7.00 |
| (from Example 300) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 315

### Chewing Gum Composition Containing Encapsulated Sodium Fluoride (NaF)

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated NaF(from Example 301) | 0.40 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 316

### Chewing Gum Composition Containing Encapsulated Calcium peroxide

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Calcium peroxide(from Example 302) | 3.40 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 317

### Chewing Gum Composition Containing Encapsulated Zinc chloride

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Zinc chloride(from Example 303) | 1.10 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 318

### Chewing Gum Composition Containing Encapsulated Carbamide peroxide

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated carbamide peroxide(from Example 304) | 3.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 319

### Chewing Gum Composition Containing Encapsulated Potassium Nitrate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Potassium Nitrate(from Example 305) | 6.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 319

### Chewing Gum Composition Containing Encapsulated Chlorhexidine

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated chlorehexidine(from Example 306) | 6.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 320

### Chewing Gum Composition Containing Encapsulated Sodium stearate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated sodium stearate(from Example 307) | 3.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 321

### Chewing Gum Composition Containing Encapsulated Sodium bicarbonate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated sodium bicarbonate(from Example 308) | 4.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 321

### Chewing Gum Composition Containing Encapsulated Cetylprydinium chloride (CPC)

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated CPC (from Example 309) | 0.90 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 322

### Chewing Gum Composition Containing Encapsulated Recaldent

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Recaldent(from Example 310) | 4.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 323

### Chewing Gum Composition Containing Encapsulated Sodium ricinoleate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated sodium ricinoleate(from Example 311) | 2.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 324

### Chewing Gum Composition Containing Encapsulated Sodium hexametaphosphate (Sodiumhexamataphosphate)

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodiumhexamataphosphate | 5.00 |
| (from Example 312) | |
| Encapsulated sucralose (from example 23) | 0.90 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Using encapsulated sucralose with encapsulated Sodiumhexamataphosphate will result in controlled release of sucralose and Sodiumhexamataphosphate. This will result in masking of saltiness taste from Sodiumhexamataphosphate release.

### Example 325

### Chewing Gum Composition Containing Encapsulated Urea

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Urea (from Example 313) | 5.00 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 326

### Chewing Gum Composition Containing Sodium tripolyphosphate (Sodiumtripolyphospate)

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Sodiumtripolyphosphate | 2.80 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Ingredient Examples of Multiple Oral Care Ingredients in a Delivery System

### Example 350

### Encapsulation of Sodiumtripolyphospate (STP) and Sodium stearate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodiumtripolyphosphate | 20.00% |
| Sodium stearate | 10.00% |
| Sucralose | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 351

### Encapsulation of Sodium Fluoride and Sodiumtripolyphosphate—Polyvinyl acetate matrix

Composition:

| Ingredient | Weight percent |
|---|---|
| Polyvinyl Acetate | 57.00% |
| Hydrogenated Oil | 3.75% |
| Glycerol Monostearate | 1.25% |
| Sodiumtripolyphosphate | 25.00% |
| Sodium Fluoride | 3.00% |
| Sucralose | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 352

### Encapsulation of Calcium peroxide and Sodiumhexamataphosphate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Calcium Peroxide | 7.00% |
| Sodiumhexamataphosphate | 23.00% |
| Sucralose | 10.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 353

### Encapsulation of Zinc Chloride and Sodiumtripolyphosphate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Zinc Chloride | 4.00% |
| Sodiumtripolyphosphate | 26.00% |
| Aspartame | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 354

### Encapsulation of Carbamide peroxide and Sodiumtripolyphosphate in Polyvinylacetate encapsulation

Composition:

| Ingredient | Weight percent |
|---|---|
| Polyvinyl Acetate | 55.00% |
| Hydrogenated Oil | 3.75% |
| Glycerol Monostearate | 1.25% |
| Sodiumtripolyphosphate | 20.00% |
| Carbamide Peroxide | 10.00% |
| Sucralose | 10.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 355

### Encapsulation of Potassium Nitrate (KNO3) and Sodiumtripolyphosphate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Potassium Nitrate | 10.00% |
| Sodiumtripolyphosphate | 20.00% |
| Sucralose | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 356

### Encapsulation of Chlorhexidine, Sodiumtripolyphosphate and Sodium Fluoride—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Chlorhexidine | 4.00% |
| Sodiumtripolyphosphate | 23.00% |
| Sodium Fluoride | 3.00% |
| Aspartame | 10.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 357

### Encapsulation of Sodium stearate, Sodiumtripolyphosphate and Menthol—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium stearate | 4.00% |
| Sodiumtripolyphosphate | 19.00% |
| Menthol | 7.00% |
| Sucralose | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 358

### Encapsulation of Sodium Bicarbonate, Sodiumtripolyphosphate and Sodium stearate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium stearate | 4.00% |
| Sodiumtripolyphosphate | 19.00% |
| Sodium bicarbonate | 7.00% |
| Sucralose | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 359

### Encapsulation of Cetylpridinium chloride (CPC), Sodium Fluoride and Sodiumtripolyphosphate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Cetylpridinium chloride | 4.00% |
| Sodiumtripolyphosphate | 23.00% |
| Sodium Fluoride | 3.00% |
| Sucralose | 10.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 360

### Encapsulation of Calcium Casein Peptone-Calcium Phosphate CCP-CP (Recaldent) and Sodiumtripolyphosphate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Recaldent | 10.00% |
| Sodiumtripolyphosphate | 20.00% |
| Sucralose | 10.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 361

### Encapsulation of Sodium ricinoleate and Sodiumtripolyphosphate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium Ricinoleate | 4.00% |
| Sodiumtripolyphosphate | 26.00% |
| Aspartame | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 362

### Encapsulation of Sodium hexametaphosphate (SHMP) and Sodium stearate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Sodium Hexametaphosphate | 26.00% |
| Sodium stearate | 4.00% |
| Sucralose | 10.00% |
| Total | 100.00% |

Procedure: Polyvinyl acetate is melted at a temperature of about 110° C. in a high shear mixer such as extruder (single or twin screw) or sigma or Banbury mixer. The hydrogenated oil and Glycerol monostearate are then added to the molten polyvinyl acetate. Sodiumhexamataphosphate is then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 363

### Encapsulation of Urea and Sodiumtripolyphosphate—Polyvinyl acetate matrix

Composition:

| Ingredient | Weight percent |
|---|---|
| Polyvinyl Acetate | 55.00% |
| Hydrogenated Oil | 3.75% |
| Glycerol Monostearate | 1.25% |
| Urea | 10.00% |
| Sodiumtripolyphosphate | 20.00% |
| Sucralose | 10.00% |
| Total | 100.00% |

Procedure: Polyvinyl acetate is melted at a temperature of about 80° C. in a high shear mixer such as extruder (single or twin screw) or sigma or Banbury mixer. The hydrogenated oil and Glycerol monostearate are then added to the molten polyvinyl acetate. Actives are then added to the resulting mixture and mixed under high shear to completely disperse the ingredients. The resulting filled polymer melt is cooled and ground to produce a powdered material with a particle size of less than 420 microns. The encapsulated matrix is stored in air tight containers with low humidity below 35° C.

### Example 364

### Chewing Gum Composition Containing Encapsulated Sodiumtripolyphosphate and Sodium stearate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodiumtripolyphosphate and | 7.00 |
| Sodium stearate (from Example 350) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 365

### Chewing Gum Composition Containing Encapsulated Sodium Fluoride and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodium Fluoride and | 5.00 |
| Sodiumtripolyphosphate (from Example 351) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 366

### Chewing Gum Composition Containing Encapsulated Calcium peroxide and Sodiumhexamataphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Calcium peroxide | 5.00 |
| and Sodiumhexamataphosphate | |
| (from Example 352) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 367

### Chewing Gum Composition Containing Encapsulated Zinc chloride and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Zinc chloride | 5.00 |
| and Sodiumtripolyphosphate | |
| (from Example 353) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 368

### Chewing Gum Composition Containing Encapsulated Carbamide peroxide and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated carbamide peroxide and | 3.00 |
| Sodiumtripolyphosphate(from Example 354) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 369

### Chewing Gum Composition Containing Encapsulated Potassium Nitrate and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Potassium Nitrate and | 6.00 |
| Sodiumtripolyphosphate (from Example 355) | |
| Total | 100.00 |

### Example 72

### Cinnamon Chewing Gum Composition Containing Sucralose (Fast Sucralose Release Gum)

| Ingredient | Weight percent |
|---|---|
| Gum Base | 36.00 |
| Sorbitol | 60.55 |
| Glycerin | 1.00 |
| Cinnamon Flavor blend | 1.90 |
| Sucralose | 0.55 |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Chew out-release studies of this gum shows faster release as compared to gum in example 73 as shown in FIG. 1.

### Example 73

### Cinnamon Chewing Gum Composition Containing Sucralose/Polyvinyl acetate matrix (from Example 23) (Controlled Sucralose Release Gum)

| Ingredient | Weight percent |
|---|---|
| Gum Base | 36.00 |
| Sorbitol | 58.95 |
| Glycerin | 1.00 |
| Cinnamon Flavor blend | 1.90 |
| Sucralose | 0.15 |
| Sucralose/polyvinyl acetate matrix | 2.00 |
| (from example 23) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged. Chew out-release studies of this gum shows controlled/slowest release as compared to gums in example 72 and 73 as shown in FIG. 1.

### Example 74

### Cinnamon Chewing Gum Composition Containing Multiple Encapsulated Sucralose/Polyvinyl acetate matrix (from Example 24) (Slowest Release Sucralose Gum)

| Ingredient | Weight percent |
|---|---|
| Gum Base | 36.00 |
| Sorbitol | 58.10 |
| Glycerin | 1.00 |
| Cinnamon Flavor | 1.90 |
| Sucralose | 0.15 |
| Sucralose/polyvinyl acetate matrix | 2.85 |
| (from example 24) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 370

### Chewing Gum Composition Containing Encapsulated Chlorhexidine Sodiumtripolyphosphate and Sodium Fluoride

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated chlorehexidine, | 6.00 |
| Sodiumtripolyphosphate and Sodium | |
| Fluoride (from Example 356) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 371

### Chewing Gum Composition Containing Encapsulated Sodium stearate, Menthol and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated sodium stearate, menthol and | 6.00 |
| Sodiumtripolyphosphate (from Example 357) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 372

### Chewing Gum Composition Containing Encapsulated Sodium bicarbonate, Sodiumtripolyphosphate and Sodium stearate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodium bicarbonate, | 6.00 |
| Sodiumtripolyphosphate and Sodium | |
| stearate (from Example 358) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 373

### Chewing Gum Composition Containing Encapsulated Cetylprydinium chloride (CPC), Sodium Fluoride and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated CPC, Sodium Fluoride and | 4.00 |
| Sodiumtripolyphosphate (from Example 359) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 374

### Chewing Gum Composition Containing Encapsulated Recaldent and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Recaldent and | 4.00 |
| Sodiumtripolyphosphate (from | |
| Example 360) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 375

### Chewing Gum Composition Containing Encapsulated Sodium ricinoleate and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodium ricinoleate and | 4.00 |
| Sodiumtripolyphosphate (from Example 361) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 376

### Chewing Gum Composition Containing Encapsulated Sodium hexametaphosphate and Sodium stearate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Sodiumhexamataphosphate | 5.00 |
| and sodium stearate (from Example 362) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

### Example 377

### Chewing Gum Composition Containing Encapsulated Urea and Sodiumtripolyphosphate

| Ingredient | Weight percent |
|---|---|
| Gum Base | 39.00 |
| Sorbitol | QS |
| Mannitol | 9.00 |
| Flavor | 3.67 |
| Glycerin | 1.50 |
| Lecithin | 0.20 |
| Aspartame | 0.30 |
| AceK | 0.15 |
| Encapsulated Urea and | 5.00 |
| Sodiumtripolyphosphate | |
| (from Example 363) | |
| Total | 100.00 |

Procedure: Gum is prepared in the following manner: The gum base is melted in a mixer. The remaining ingredients are added to the molten gum base. The melted gum base with ingredients are mixed to completely disperse the ingredients. The resulting chewing gum is allowed to cool. The cooled chewing gum is sized and conditioned for about a week and packaged.

1-36. (canceled)

37. A delivery system for use in an edible composition, said delivery system comprising: an encapsulating material; and at least one ingredient encapsulated with said encapsulating material, wherein said delivery system has at least one managed release characteristic.

38. The delivery system of claim 37, wherein said at least one managed release characteristic is selected from the group consisting of tensile strength of said encapsulated at least one ingredient, hydrophobicity of said encapsulating material, average particle size of said delivery system, ratio of said at least one ingredient to said encapsulating material, pre-treatment of said at least one ingredient, post-treatment of said encapsulated at least one ingredient, and combinations thereof.

39. The delivery system of claim 38, wherein said tensile strength of said encapsulated at least one ingredient is selected from the group consisting of at least about 6,500 psi, at least about 10,000 psi, and at least about 20,000 psi.

40. The delivery system of claim 38, wherein said average particle size of said delivery system is selected from the group consisting of less than 710, and less than 420.

41. The delivery system of claim 38, wherein said hydrophobicity of said encapsulating material as measured by water absorption is selected from the group consisting of from about 0 to about 15%, from about 15% to about 50%, and from about 50% to about 100% by weight.

42. The delivery system of claim 38, wherein said ratio of said at least one ingredient to said encapsulating material is selected from the group consisting of from about 1 to about 99, and from about 30 to about 70.

43. The delivery system of claim 38, wherein said pre-treatment of said at least one ingredient includes a coating at least partially surrounding said ingredient.

44. The delivery system of claim 38, wherein said post-treatment of said encapsulated at least one ingredient includes a coating at least partially surrounding said encapsulated at least one ingredient.

45. The delivery system of claim 37, wherein said at least one release characteristic includes a first release characteristic and a second release characteristic.

46. The delivery system of claim 45, wherein said first release characteristic is tensile strength of said encapsulated ingredient and said second release characteristic is selected from the group consisting of hydrophobicity of said encapsulating material, particle size of said delivery system, ratio of said ingredient to said encapsulating material, pre-treatment of said ingredient, post-treatment of said delivery system, and combinations thereof.

47. The delivery system of claim 45, wherein said first release characteristic is hydrophobicity of said encapsulating material and said second release characteristic is selected from the group consisting of particle size of said delivery system, ratio of said ingredient to said encapsulating material, pre-treatment of said ingredient, post-treatment of said delivery system, and combinations thereof.

48. The delivery system of claim 45, wherein said first release characteristic is particle size of said delivery system and said second release characteristic is selected from the group consisting of ratio of said ingredient to said encapsulating material, pre-treatment of said ingredient, post-treatment of said delivery system, and combinations thereof.

49. The delivery system of claim 45, wherein said first release characteristic is ratio of said ingredient to said encapsulating material and said second release characteristic is selected from the group consisting of pre-treatment of said ingredient, post-treatment of said delivery system, and combinations thereof.

50. The delivery system of claim 45, wherein said first release characteristic is pre-treatment of said ingredient and said second release characteristic is post-treatment of said delivery system.

51. A delivery system for use in an edible composition, said delivery system comprising: an encapsulating material; and at least one ingredient encapsulated with said encapsulating material, wherein said delivery system has at least three managed release characteristics.

52. The delivery system of claim 51, wherein said at least three managed release characteristics are selected from the group consisting of tensile strength of said encapsulated at least one ingredient, hydrophobicity of said encapsulating material, average particle size of said delivery system, ratio of said at least one ingredient to said encapsulating material, pre-treatment of said at least one ingredient, post-treatment of said encapsulated at least one ingredient, and combinations thereof.

53. An edible composition comprising: a first delivery system comprising a first encapsulating material and at least one first ingredient encapsulated with said first encapsulating material; and a second delivery system comprising a second encapsulating material and an ingredient encapsulated with said second encapsulating material.

54. The edible composition of claim 53, wherein said ingredient is selected from the group consisting of sweeteners, flavors, sensates, functional ingredients, and combinations thereof.

55. The delivery system of claim 53, wherein said first encapsulating material and said second encapsulating material are the same.

56. The delivery system of claim 53, wherein said first delivery system has at least one first managed release characteristic.

57. The delivery system of claim 53, wherein said second delivery system has at least one second managed release characteristic.

58. The delivery system of claim 56, wherein said at least one first managed release characteristic is selected from the group consisting of tensile strength of said encapsulated at least one ingredient, hydrophobicity of said encapsulating material, average particle size of said delivery system, ratio of said at least one ingredient to said encapsulating material, pre-treatment of said at least one ingredient, post-treatment of said encapsulated at least one ingredient, and combinations thereof.

59. The delivery system of claim 57, wherein said at least one second managed release characteristic is selected from the group consisting of tensile strength of said encapsulated at least one ingredient, hydrophobicity of said encapsulating material, average particle size of said delivery system, ratio of said at least one ingredient to said encapsulating material, pre-treatment of said at least one ingredient, post-treatment of said encapsulated at least one ingredient, and combinations thereof.

60. The delivery system of claim 53, wherein said first delivery system and said second delivery system are encapsulated together by a third encapsulating material.
